SEQUENCE LISTING (108 sequences)

SEQ ID NO: 1 - protein, 1368 aa, synthetic construct
  REGION 1..1368: note = dCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1053 aa, synthetic construct
  REGION 1..1053: note = Staphylococcus aureus dCas9 (Mutant: D10A and H557A)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1053 aa, synthetic construct
  REGION 1..1053: note = Staphylococcus aureus dCas9 (Mutant: D10A and N580A)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVHAIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1053 aa, synthetic construct; identical to SEQ ID NO: 3
  REGION 1..1053: note = Streptococcus pyogenes dCas9 (D10A, H840A)

SEQ ID NO: 5 - protein, 984 aa, synthetic construct
  REGION 1..984: note = Campylobacter jejuni dCas9 (D8A, H559A)
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 6 - protein, 1084 aa, synthetic construct
  REGION 1..1084: note = Corynebacterium diphtheria dCas9 (D8A, H573A)
MKYHVGIAVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDAIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 7 - protein, 1107 aa, synthetic construct
  REGION 1..1107: note = Eubacterium ventriosum dCas9 (D8A, H592A)
MGYTVGLAIGVASVGVAVLDENDNIVEAVSNIFDEADTSNNKVRRTLREGRRTKRRQKTRIEDFKQLWETSGYIIPHKLHLNIIELRNKGLTELLSLDELYCVLLSMLKHRGISYLEDADDGEKGNAYKKGLAFNEKQLKEKMPCEIQLERMKKYGKYHGEFIIEINDEKEYQSNVFTTKAYKKELEKIFETQRCNGNKINTKFIKKYMEIYERKREYYIGPGNEKSRTDYGIYTTRTDEEGNFIDEKNIFGKLIGKCSVYPEEYRASSASYTAQEFNLLNDLNNLKINNEKLTEFQKKEIVEIIKDASSVNMRKIIKKVIDEDIEQYSGARIDKKGKEIYHTFEIYRKLKKELKTINVDIDSFTREELDKTMDILTLNTERESIVKAFDEQKFVYEENLIKKLIEFRKNNQRLFSGWHSFSYKAMLQLIPVMYKEPKEQMQLLTEMNVFKSKKEKYVNYKYIPENEVVKEIYNPVVVKSIRTTVKILNALIKKYGYPESVVIEMPRDKNSDDEKEKIDMNQKKNQEEYEKILNKIYDEKGIEITNKDYKKQKKLVLKLKLWNEQEGLCLYSGKKIAIEDLLNHPEFFEIDAIIPKSISLDDSRSNKVLVYKTENSIKENDTPYHYLTRINGKWGFDEYKANVLELRRRGKIDDKKVNNLLCMEDITKIDVVKGFINRNLNDTRYASRVVLNEMQSFFESRKYCNTKVKVIRGSLTYQMRQDLHLKKNREESYSHHAVDAMLIAFSQKGYEAYRKIQKDCYDFETGEILDKEKWNKYIDDDEFDDILYKERMNEIRKKIIEAEEKVKYNYKIDKKCNRGLCNQTIYGTREKDGKIHKISSYNIYDDKECNSLKKMINSGKGSDLLMYNNDPKTYRDMLKILETYSSEKNPFVAYNKETGDYFRKYSKNHNGPKVEKVKYYSGQINSCIDISHKYGHAKNSKKVVLVSLNPYRTDVYYDNDTGKYYLVGVKYNHIKCVGNKYVIDSETYNELLRKEGVLNSDENLEDLNSKNITYKFSLYKNDIIQYEKGGEYYTERFLSRIKEQKNLIETKPINKPNFQRKNKKGEWENTRNQIALAKTKYVGKLVTDVLGNCYIVNMEKFSLVVDK

SEQ ID NO: 8 - protein, 1130 aa, synthetic construct
  REGION 1..1130: note = Streptococcus pasteurianus dCas9 (D10A, H599A)
MTNGKILGLAIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGAKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 9 - protein, 1125 aa, synthetic construct
  REGION 1..1125: note = Lactobacillus farciminis dCas9 (D10A, H611A)
MTDRISLGLAIGVASVGFSVLDIDKGKVIELGARLFNATVAAGNQDRRDMRGSRRLLNRNKQRRKDVSKLFQEYGLLDNFDRDNFHTAFNNNWNPYELRVKGLTKQLNKEELADSLYQIIKRRGISYALKDADANEDGTDYSSSLKINSQELIEKTPAQIQLQRLNDYGKVRGKVIVGDDIDDQKVLLNVFPTNAYEKEARQIIATQQQFYPEILTGEFVKEYCQILTRKRDYFVGPGNEKSRTDYGIYKTDGRTLDNLFEELIGHDKIYPDELRASAASYTAQLFNVLNDLNNLRILSYEDGKLTQIDKEKIIAEIKNNTTVVNMLNVIKKVTGCQKDDIKGFRLNEKDKPEISSMPIYRKAHKDFLKVGIDITDWPIDFIDRLSFILTLNTENGEIRKQINKRLVPDFDFLNDDLVQLIIDNKDSFDIKTNNKWHRFSLKTMNKLIPTMIERPVEQMTLLTEMGLIKKDTKRFEGNKYLPYKEIANDIFNPVASKSVREALKIVNAVLKKYGHIEYIVIEMPRDKNLDEERKQIADFQKKNKKIKDAAFNAFVKAVGSKKNVKIALSKNRKLQMGIRLWHQQQGIDPYNGEEINANDLISNPDKFEIDAIIPQSISFDDGINNKTLCFASMNQVKGQKTPYEFLNEGHGQGYAKFKAIVSKNKNFSKAKRDNYLFEENVSNIETRKRFLSRNLVDTRYSSRVVLNSLQEFFHEKSDDTKVTVIRGKFTSNMRKHWHIDKTRDTFHHHAIDASIIAATPFLRMWKKGSTIFPTKINETSIDIETGEILDDKNFDKVMYEEPYSGFISEIMNADDRIKFSHQVDKKMNRKVSDATIYSTRIGKLSKDKKDAEYVVAKVKDIYSVDGYENFKKVYKKDKTKFLLYKYDPRTFSELEKIVIDYPDKIEKVQTNGKIKAVDISPFELYRRDNGMVRKYSKKTGPAIKQLKYLDKKLGSHIDITPKNANNKHVVLQSLKPWRTDVYLNHETGEYEIMGIKYSDLKFNKNEGYGIKKDKYLKIKNIEGVSENSEFMFSLYRKDRIKVQDMETNESVELLFWSRNFSNKKYAELKPISQIDNDKVLPIYGRGRLIKRLIPKNCKIWKVNTSILGDTYYLEKESNYPQKILD

SEQ ID NO: 10 - protein, 1179 aa, synthetic construct
  REGION 1..1179: note = Sphaerochaeta globosa dCas9 (D30A, H635A)
MSKKVSRRYEEQAQEICQRLGSRPYSIGLALGVGSIGVAVAAYDPIKKQPSDLVFVSSRIFIPSTGAAERRQKRGQRNSLRHRANRLKFLWKLLAERNLMLSYSEQDVPDPARLRFEDAVVRANPYELRLKGLNEQLTLSELGYALYHIANHRGSSSVRTFLDEEKSSDDKKLEEQQAMTEQLAKEKGISTFIEVLTAFNTNGLIGYRNSESVKSKGVPVPTRDIISNEIDVLLQTQKQFYQEILSDEYCDRIVSAILFENEKIVPEAGCCPYFPDEKKLPRCHFLNEERRLWEAINNARIKMPMQEGAAKRYQSASFSDEQRHILFHIARSGTDITPKLVQKEFPALKTSIIVLQGKEKAIQKIAGFRFRRLEEKSFWKRLSEEQKDDFFSAWTNTPDDKRLSKYLMKHLLLTENEVVDALKTVSLIGDYGPIGKTATQLLMKHLEDGLTYTEALERGMETGEFQELSVWEQQSLLPYYGQILTGSTQALMGKYWHSAFKEKRDSEGFFKPNTNSDEEKYGRIANPVVHQTLNELRKLMNELITILGAKPQEITVELARELKVGAEKREDIIKQQTKQEKEAVLAYSKYCEPNNLDKRYIERFRLLEDQAFVCPYCLEHISVADIAAGRADVDAIFPRDDTADNSYGNKVVAHRQCNDIKGKRTPYAAFSNTSAWGPIMHYLDETPGMWRKRRKFETNEEEYAKYLQSKGFVSRFESDNSYIAKAAKEYLRCLFNPNNVTAVGSLKGMETSILRKAWNLQGIDDLLGSRHWSKDADTSPTMRKNRDDNRHHGLDAIVALYCSRSLVQMINTMSEQGKRAVEIEAMIPIPGYASEPNLSFEAQRELFRKKILEFMDLHAFVSMKTDNDANGALLKDTVYSILGADTQGEDLVFVVKKKIKDIGVKIGDYEEVASAIRGRITDKQPKWYPMEMKDKIEQLQSKNEAALQKYKESLVQAAAVLEESNRKLIESGKKPIQLSEKTISKKALELVGGYYYLISNNKRTKTFVVKEPSNEVKGFAFDTGSNLCLDFYHDAQGKLCGEIIRKIQAMNPSYKPAYMKQGYSLYVRLYQGDVCELRASDLTEAESNLAKTTHVRLPNAKPGRTFVIIITFTEMGSGYQIYFSNLAKSKKGQDTSFTLTTIKNYDVRKVQLSSAGLVRYVSPLLVDKIEKDEVALCGE

SEQ ID NO: 11 - protein, 1168 aa, synthetic construct
  REGION 1..1168: note = Azospirillum (strain B510) dCas9 (D75A, H680A)
MARPAFRAPRREHVNGWTPDPHRISKPFFILVSWHLLSRVVIDSSSGCFPGTSRDHTDKFAEWECAVQPYRLSFALGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDAILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS

SEQ ID NO: 12 - protein, 1003 aa, synthetic construct
  REGION 1..1003: note = Gluconacetobacter diazotrophicus dCas9 (D11A, H587A)
MIDESLTFGIALGIGSCGWAVLRRPSAFGRKGVIEGMGSWCFDVPETSKERTPTNQIRRSNRLLRRVIRRRRNRMAAIRRLLHAAGLLPSTDSDALKRPGHDPWELRARGLDKPLKPVEFAVVLGHIAKRRGFKSAAKRKATNISSDDKKMLTALEATRERLGRYRTVGEMFARDPDFASRRRNREGKYDRTTARDDLEHEVHALFAAQRRLGQGFASPELEEAFTASAFHQRPMQDSERLVGFCPFERTEKRAAKLTPSFERFRLLARLLNLRITTPDGERPLTVDEIALVTRDLGKTAKLSIKRVRTLIGLEDNQRFTTIRPEDEDRDIVARTGGAMTGTATLRKALGEALWTDMQERPEQLDAIVQVLSFFEANETITEKLREIGLTLAVLDVLLTALDAGVFAKFKGAAHISTKAARNLLPHLEQGRRYDEACTMAGYDHAASRLSHHGQIVAKTQFNALVTEIGESIANPIARKALIEGLKQIWAMRNHWGLPGSIHVELARDVGNSIEKRREIEKHIEKNTALRARERREVHDLLDLEDVNGDTLLRYRLWKEQGGKCLYTGKAIHIRQIAATDNSVQVDAILPWSRFGDDSFNNKTLCLASANQQKKRSTPYEWLSGQTGDAWNAFVQRIETNKELRGFKKRNYLLKNAKEAEEKFRSRNLNDTRYAARLFAEAVKLLYAFGERQEKGGNRRVFTRPGALTAALRQAWGVESLKKQDGKRINDDRHHALDALTVAAVDEAEIQRLTKSFHEWEQQGLGRPLRRVEPPWESFRADVEATYPEVFVARPERRRARGEGHAATIRQVKERECTPIVFERKAVSSLKEADLERIKDGERNEAIVEAIRSWIATGRPADAPPRSPRGDIITKIRLATTIKAAVPVRGGTAGRGEMVRADVFSKPNRRGKDEWYLVPVYPHQIMNRKAWPKPPMRSIVANKDEDEWTEVGPEHQFRFSLYPRSNIEIIRPSGEVIEGYFVGLHRNTGALIPTPVGPDSYVIA

SEQ ID NO: 13 - protein, 1082 aa, synthetic construct
  REGION 1..1082: note = Neisseria cinerea dCas9 (D16A, H588A)
MAAFKPNPMNYILGLAIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDAALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 14 - protein, 1140 aa, synthetic construct
  REGION 1..1140: note = Roseburia intestinalis dCas9 (D24A, H628A)
MRENGSDERRRNMDEKMDYRIGLAIGIASVGWAVLQNNSDDEPVRIVDLGVRIFDTAEIPKTGESLAGPRRAARTTRRRLRRRKHRLDRIKWLFENQGLINIDDFLKRYNMAGLPDVYQLRYEALDRKLTDEELAQVLLHIAKHRGFRSTRKAETAAKENGAVLKATDENQKRMQEKGYRTVGEMIYLDEAFRTGCSWSEKGYILTPRNKAENYQHTMLRAMLVEEVKEIFSSQRRLGNEKATEELEEKYLEIMTSQRSFDLGPGMQPDGKPSPYAMEGFSDRVGKCTFLGDQGELRGAKGTYTAEYFVALQKINHTKLVNQDGETRNFTEEERRALTLLLFTQKEVKYAAVRKKLGLPEDILFYNLNYKKAATKEEQQKENQNTEKAKFIGMPYYHDYKKCLEERVKYLTENEVRDLFDEIGMILTCYKNDDSRTERLAKLGLVPIEMEGLLAYTPTKFQHLSMKAMRNIIPFLEKGMTYDKACEEAGYDFKADSKGTKQKLLTGENVNQTINEITNPVVKRSVSQTVKVINAIIRTYGSPQAINIELAREMSKTFEERRKIKGDMEKRQKNNEDVKKQIQELGKLSPTGQDILKYRLWQEQQGICMYSGKTIPLEELFKPGYDIDAILPYSITFDDSFRNKVLVTSQENRQKGNRTPYEYMGNDEQRWNEFETRVKTTIRDYKKQQKLLKKHFSEEERSEFKERNLTDTKYITTVIYNMIRQNLEMAPLNRPEKKKQVRAVNGAITAYLRKRWGLPQKNRETDTHHAMDAVVIACCTDGMIQKISRYTKVRERCYSKGTEFVDAETGEIFRPEDYSRAEWDEIFGVHIPKPWETFRAELDVRMGDDPKGFLDTHSDVALELDYPEYIYENLRPIFVSRMPNHKVTGAAHADTIRSPRHFKDEGIVLTKTALTDLKLDKDGEIDGYYNPQSDLLLYEALKKQLLLYGNDAKKAFAQDFHKPKADGTEGPVVRKVKIQKKQTMGVFVDSGNGIAENGGMVRIDVFRVNGKYYFVPVYTADVVKKVLPNRASTAHKPYGEWKVMEDKDFLFSLYSRDLIHIKSKKDIPIKMVNGGMEGIKETYAYYIGADISAANIQGIAHDSRYKFRGLGIQSLDVLEKCQIDVLGHVSVVRSEKRMGFS

SEQ ID NO: 15 - protein, 1037 aa, synthetic construct
  REGION 1..1037: note = Parvibaculum lavamentivorans dCas9 (D8A, H601A)
MERIFGFAIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEAIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 16 - protein, 1132 aa, synthetic construct
  REGION 1..1132: note = Nitratifractor salsuginis (strain DSM 16511) dCas9 (D8A, H611A)
MKKILGVALGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEAIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 17 - protein, 997 aa, synthetic construct
  REGION 1..997: note = Campylobacter lari dCas9 (D7A, H567A)
MKILGFAIGINSIGWAFVENDELKDCGVRIFTEAENPSNKESLALPRRNARSSRRRLKRRKARLVAIKRILAKELKLNFKDYVAADGELPKAYEGKLTSIYELRYKALIQQLETKDLARVILHIAKHRGYMNKNEKKSNDTKKGKILSALKNNALKLENYQSVGEYFYKEFFQKYKENTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEILNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYYEACEITNLKPKTVDEKEDFLPAFCDSIFAHELSNPVVNRAISEYRKVLNALLKKYGKVHKIHLELARDVGLSKKAREKIEDQQKNNKAINDWALKECENIGIKASAKNILKLKLWKEQKEICIYSGNKISIEHLKDEKALEVDAIYPYSRSFDDSFINKVLVFTKENQEKLNKTPFEAFGKNIEKWSKIQTLAQNLPYKKKNKILDENFKDKQQQDFISRNLNDTRYITTLIAKYTKEYLNFLPLSENENTNLKSGEKGSKIHIQTISGMLTSVLRHTWGFDKKDRNNHLHHALDAIIVAYSTSSIIKAFSDFKKNQELLKARFYAKELTSDNYKHQVKFFEPFKGFREKILSKIDEIFVSTPPRKRARGALHEETFYSEDKMIKKYNSKEGLQIALSCGRVRKIGTKYVENDTMVRVDIFKKQNKFYAIPIYVMDFALGILPNKIVITGKDKNNNPKQWQTIDESYEFCFSLYKNDLILLQKKNMQEPEFAYYNSFDIDRSRIKIKKHDNKFENLTSNQKLLFTNKDKGKNRTGIQNLKIFEKYIVTPLGDKIKADFQPRENISLKTSKKHGL

SEQ ID NO: 18 - protein, 1388 aa, synthetic construct
  REGION 1..1388: note = Streptococcus thermophilus (strain LMD-9) dCas9 (D10A, H847A)
MTKPYSIGLAIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDAIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 19 - protein, 27 aa, synthetic construct
  REGION 1..27: note = Linker
SSGNSNANSRGPSFSSGLVPLSLRGSH

SEQ ID NO: 20 - protein, 80 aa, synthetic construct
  REGION 1..80: note = XTEN80
GGPSSGAPPPSGGSPAGSPTSTEEGTSESATPESGPGTSTEPSEGSAPGSPAGSPTSTEEGTSTEPSEGSAPGTSTEPSE

SEQ ID NO: 21 - protein, 16 aa, synthetic construct
  REGION 1..16: note = XTEN16
SGSETPGTSESATPES

SEQ ID NO: 22 - protein, 62 aa, synthetic construct
  REGION 1..62: note = KRAB
RTLVTFKDVFVDFTREEWKLLDTAQQIVYRNVMLENYKNLVSLGYQLTKPDVILRLEKGEEP

SEQ ID NO: 23 - protein, 302 aa, synthetic construct
  REGION 1..302: note = DNMT3A
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 24 - protein, 215 aa, synthetic construct
  REGION 1..215: note = DNMT3L
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 25 - protein, 16 aa, synthetic construct
  REGION 1..16: note = NLS
APKKKRKVGIHGVPAA

SEQ ID NO: 26 - protein, 16 aa, synthetic construct
  REGION 1..16: note = NLS
KRPAATKKAGQAKKKK

SEQ ID NO: 27 - DNA, 20 bp, synthetic construct
  misc_feature 1..20: note = mouse PCSK9 sgRNA1
  source 1..20: mol_type = other DNA
tggacgcgcaggctgccggt

SEQ ID NO: 28 - DNA, 20 bp, synthetic construct
  misc_feature 1..20: note = mouse PCSK9 sgRNA2
  source 1..20: mol_type = other DNA
ccaccttcacgtggacgcgc

SEQ ID NO: 29 - DNA, 20 bp, synthetic construct
  misc_feature 1..20: note = mouse PCSK9 sgRNA3
  source 1..20: mol_type = other DNA
gtggacgcgcaggctgccgg

SEQ ID NO: 30 - DNA, 20 bp, synthetic construct
  misc_feature 1..20: note = mouse PCSK9 sgRNA4
  source 1..20: mol_type = other DNA
ctctctctttctgaggctag

SEQ ID NO: 31 - DNA, 20 bp, synthetic construct
  misc_feature 1..20: note = mouse PCSK9 sgRNA5
  source 1..20: mol_type = other DNA
cacgtggacgcgcaggctgc

SEQ ID NO: 32 - DNA, 20 bp, synthetic construct
  misc_feature 1..20: note = mouse PCSK9 sgRNA6
  source 1..20: mol_type = other DNA
ttaagaggggggaatgtaac

SEQ ID NO: 33 - DNA, 20 bp, synthetic construct
  misc_feature 1..20: note = mouse PCSK9 sgRNA7
  source 1..20: mol_type = other DNA
aacctgatcctttagtaccg

SEQ ID NO: 34 - DNA, 20 bp, synthetic construct
  misc_feature 1..20: note = mouse PCSK9 sgRNA8
  source 1..20: mol_type = other DNA
tcagagaggatcttccgatg

SEQ ID NO: 35 - DNA, 20 bp, synthetic construct
  misc_feature 1..20: note = mouse PCSK9 sgRNA9
  source 1..20: mol_type = other DNA
ggatcttccgatggggctcg

SEQ ID NO: 36 - DNA, 20 bp, synthetic construct
  misc_feature 1..20: note = mouse PCSK9 sgRNA10
  source 1..20: mol_type = other DNA
gcgtcatttgacgctgtctg

SEQ ID NO: 37 - DNA, 20 bp, synthetic construct
  misc_feature 1..20: note = mouse PCSK9 sgRNA11
  source 1..20: mol_type = other DNA
tcatttgacgctgtctgggg

SEQ ID NO: 38 - DNA, 20 bp, synthetic construct
  misc_feature 1..20: note = mouse PCSK9 sgRNA12
  source 1..20: mol_type = other DNA
gatcctttagtaccggggcc

SEQ ID NO: 39 - DNA, 20 bp, synthetic construct
  misc_feature 1..20: note = mouse PCSK9 sgRNA13
  source 1..20: mol_type = other DNA
tgcagcccaattaggatttg

SEQ ID NO: 40 - DNA, 20 bp, synthetic construct
  misc_feature 1..20: note = human PCSK9 sgRNA1
  source 1..20: mol_type = other DNA
ggctcagaccctgaactgaa

SEQ ID NO: 41 - DNA, 20 bp, synthetic construct
  misc_feature 1..20: note = human PCSK9 sgRNA2
  source 1..20: mol_type = other DNA
gaggcgctcatggttgcagg

SEQ ID NO: 42 - DNA, 20 bp, synthetic construct
  misc_feature 1..20: note = human PCSK9 sgRNA3
  source 1..20: mol_type = other DNA
tcagaccctgaactgaacgg

SEQ ID NO: 43 - DNA, 20 bp, synthetic construct
  misc_feature 1..20: note = human PCSK9 sgRNA4
  source 1..20: mol_type = other DNA
aggcgctcatggttgcaggc

SEQ ID NO: 44 - DNA, 20 bp, synthetic construct
  misc_feature 1..20: note = human PCSK9 sgRNA5
  source 1..20: mol_type = other DNA
agttcagggtctgagcctgg

SEQ ID NO: 45 - DNA, 20 bp, synthetic construct
  misc_feature 1..20: note = human PCSK9 sgRNA6
  source 1..20: mol_type = other DNA
cgggcgccgccgttcagttc

SEQ ID NO: 46 - DNA, 20 bp, synthetic construct
  misc_feature 1..20: note = human PCSK9 sgRNA7
  source 1..20: mol_type = other DNA
gggcgccgccgttcagttca

SEQ ID NO: 47 - DNA, 20 bp, synthetic construct
  misc_feature 1..20: note = human PCSK9 sgRNA8
  source 1..20: mol_type = other DNA
actgcctggctcactcctcc

SEQ ID NO: 48 - DNA, 20 bp, synthetic construct
  misc_feature 1..20: note = human PCSK9 sgRNA9
  source 1..20: mol_type = other DNA
tgagcctggaggagtgagcc

SEQ ID NO: 49 - DNA, 20 bp, synthetic construct
  misc_feature 1..20: note = human PCSK9 sgRNA10
  source 1..20: mol_type = other DNA
ttcagttcagggtctgagcc

SEQ ID NO: 50 - DNA, 20 bp, synthetic construct
  misc_feature 1..20: note = human PCSK9 sgRNA11
  source 1..20: mol_type = other DNA
ccaggcagtgagactggctc

SEQ ID NO: 51 - DNA, 20 bp, synthetic construct
  misc_feature 1..20: note = human PCSK9 sgRNA12
  source 1..20: mol_type = other DNA
tgagcgcctcgacgtcgctg

SEQ ID NO: 52 - DNA, 20 bp, synthetic construct
  misc_feature 1..20: note = human PCSK9 sgRNA13
  source 1..20: mol_type = other DNA
aggtttccgcagcgacgtcg

SEQ ID NO: 53 - DNA, 20 bp, synthetic construct
  misc_feature 1..20: note = human PCSK9 sgRNA14
  source 1..20: mol_type = other DNA
gccaggcagtgagactggct

SEQ ID NO: 54 - DNA, 20 bp, synthetic construct
  misc_feature 1..20: note = human PCSK9 sgRNA15
  source 1..20: mol_type = other DNA
gtcgaggcgctcatggttgc

SEQ ID NO: 55 - DNA, 20 bp, synthetic construct
  misc_feature 1..20: note = human PCSK9 sgRNA16
  source 1..20: mol_type = other DNA
cccgagccagtctcactgcc

SEQ ID NO: 56 - DNA, 20 bp, synthetic construct
  misc_feature 1..20: note = human PCSK9 sgRNA17
  source 1..20: mol_type = other DNA
ggcagtgagactggctcggg

SEQ ID NO: 57 - DNA, 20 bp, synthetic construct
  misc_feature 1..20: note = human PCSK9 sgRNA18
  source 1..20: mol_type = other DNA
cagcgacgtcgaggcgctca

SEQ ID NO: 58 - DNA, 20 bp, synthetic construct
  misc_feature 1..20: note = human PCSK9 sgRNA19
  source 1..20: mol_type = other DNA
gcagtgagactggctcgggc

SEQ ID NO: 59 - DNA, 20 bp, synthetic construct
  misc_feature 1..20: note = human PCSK9 sgRNA20
  source 1..20: mol_type = other DNA
gagactggctcgggcgggcc

SEQ ID NO: 60 - DNA, 20 bp, synthetic construct
  misc_feature 1..20: note = human PCSK9 sgRNA21
  source 1..20: mol_type = other DNA
tgagactggctcgggcgggc

SEQ ID NO: 61 - DNA, 20 bp, synthetic construct
  misc_feature 1..20: note = human PCSK9 sgRNA22
  source 1..20: mol_type = other DNA
ggagacctagaggccgtgcg

SEQ ID NO: 62 - DNA, 20 bp, synthetic construct
  misc_feature 1..20: note = human PCSK9 sgRNA23
  source 1..20: mol_type = other DNA
cggcggcgccttgagccttg

SEQ ID NO: 63 - DNA, 20 bp, synthetic construct
  misc_feature 1..20: note = human PCSK9 sgRNA24
  source 1..20: mol_type = other DNA
gcgccttgagccttgcggtg

SEQ ID NO: 64 - DNA, 20 bp, synthetic construct
  misc_feature 1..20: note = human PCSK9 sgRNA25
  source 1..20: mol_type = other DNA
ggcgccttgagccttgcggt

SEQ ID NO: 65 - DNA, 20 bp, synthetic construct
  misc_feature 1..20: note = human PCSK9 sgRNA26
  source 1..20: mol_type = other DNA
cctccccaccgcaaggctca

SEQ ID NO: 66 - DNA, 20 bp, synthetic construct
  misc_feature 1..20: note = human PCSK9 sgRNA27
  source 1..20: mol_type = other DNA
gcacagtcctccccaccgca

SEQ ID NO: 67 - DNA, 20 bp, synthetic construct
  misc_feature 1..20: note = human PCSK9 sgRNA28
  source 1..20: mol_type = other DNA
cggcgccttgagccttgcgg

SEQ ID NO: 68 - DNA, 20 bp, synthetic construct
  misc_feature 1..20: note = human PCSK9 sgRNA29
  source 1..20: mol_type = other DNA
aggccgtgcgcggtccacgc

SEQ ID NO: 69 - DNA, 20 bp, synthetic construct
  misc_feature 1..20: note = human PCSK9 sgRNA30
  source 1..20: mol_type = other DNA
ctgtgcaggagctgaagttc

SEQ ID NO: 70 - DNA, 20 bp, synthetic construct
  misc_feature 1..20: note = human PCSK9 sgRNA31
  source 1..20: mol_type = other DNA
ccttgagccttgcggtgggg

SEQ ID NO: 71 - DNA, 20 bp, synthetic construct
  misc_feature 1..20: note = human PCSK9 sgRNA32
  source 1..20: mol_type = other DNA
gtggaccgcgcacggcctct

SEQ ID NO: 72 - DNA, 20 bp, synthetic construct
  misc_feature 1..20: note = human PCSK9 sgRNA33
  source 1..20: mol_type = other DNA
ggctcaaggcgccgccggcg

SEQ ID NO: 73 - DNA, 20 bp, synthetic construct
  misc_feature 1..20: note = human PCSK9 sgRNA34
  source 1..20: mol_type = other DNA
tgcggtggggaggactgtgc

SEQ ID NO: 74 - DNA, 20 bp, synthetic construct
  misc_feature 1..20: note = human PCSK9 sgRNA35
  source 1..20: mol_type = other DNA
cgcaaggctcaaggcgccgc

SEQ ID NO: 75 - DNA, 20 bp, synthetic construct
  misc_feature 1..20: note = human PCSK9 sgRNA36
  source 1..20: mol_type = other DNA
ccgtgcgcggtccacgccgg

SEQ ID NO: 76 - DNA, 20 bp, synthetic construct
  misc_feature 1..20: note = human PCSK9 sgRNA37
  source 1..20: mol_type = other DNA
ccgccggcgtggaccgcgca

SEQ ID NO: 77 - DNA, 20 bp, synthetic construct
  misc_feature 1..20: note = human PCSK9 sgRNA38
  source 1..20: mol_type = other DNA
ggagctgacggtgcccatga

SEQ ID NO: 78 - DNA, 20 bp, synthetic construct
  misc_feature 1..20: note = human PCSK9 sgRNA39
  source 1..20: mol_type = other DNA
tcaggagcagggcgcgtgaa

SEQ ID NO: 79 - DNA, 20 bp, synthetic construct
  misc_feature 1..20: note = human PCSK9 sgRNA40
  source 1..20: mol_type = other DNA
gggacgcgtcgttgcagcag

SEQ ID NO: 80 - DNA, 20 bp, synthetic construct
  misc_feature 1..20: note = human PCSK9 sgRNA41
  source 1..20: mol_type = other DNA
gagaggttgctgtcctggcg

SEQ ID NO: 81 - DNA, 20 bp, synthetic construct
  misc_feature 1..20: note = human PCSK9 sgRNA42
  source 1..20: mol_type = other DNA
tgaaggggcgcgcggaatcc

SEQ ID NO: 82 - DNA, 20 bp, synthetic construct
  misc_feature 1..20: note = human PCSK9 sgRNA43
  source 1..20: mol_type = other DNA
acctctcccctggccctcat

SEQ ID NO: 83 - DNA, 20 bp, synthetic construct
  misc_feature 1..20: note = human PCSK9 sgRNA44
  source 1..20: mol_type = other DNA
gcggctcccagctcccagcc

SEQ ID NO: 84 - DNA, 20 bp, synthetic construct
  misc_feature 1..20: note = human PCSK9 sgRNA45
  source 1..20: mol_type = other DNA
caaatcctaactgggctgga

SEQ ID NO: 85 - DNA, 20 bp, synthetic construct
  misc_feature 1..20: note = human PCSK9 sgRNA46
  source 1..20: mol_type = other DNA
tcccgcctctcaccctgcgt

SEQ ID NO: 86 - DNA, 20 bp, synthetic construct
  misc_feature 1..20: note = human PCSK9 sgRNA47
  source 1..20: mol_type = other DNA
ggagtctggcatcccacgca

SEQ ID NO: 87 - DNA, 20 bp, synthetic construct
  misc_feature 1..20: note = human PCSK9 sgRNA48
  source 1..20: mol_type = other DNA
aaacctgatcctccagtccg

SEQ ID NO: 88 - DNA, 20 bp, synthetic construct
  misc_feature 1..20: note = human PCSK9 sgRNA49
  source 1..20: mol_type = other DNA
gtgtgggtgcttgacgcctg

SEQ ID NO: 89 - DNA, 20 bp, synthetic construct
  misc_feature 1..20: note = human PCSK9 sgRNA50
  source 1..20: mol_type = other DNA
ttaaacattaacggaacccc

SEQ ID NO: 90 - DNA, 20 bp, synthetic construct
  misc_feature 1..20: note = human PCSK9 sgRNA51
  source 1..20: mol_type = other DNA
aacctgatcctccagtccgg

SEQ ID NO: 91 - DNA, 20 bp, synthetic construct
  misc_feature 1..20: note = human PCSK9 sgRNA52
  source 1..20: mol_type = other DNA
gccagactccaagttctgcc

SEQ ID NO: 92 - DNA, 20 bp, synthetic construct
  misc_feature 1..20: note = human PCSK9 sgRNA53
  source 1..20: mol_type = other DNA
atcccacgcagggtgagagg

SEQ ID NO: 93 - DNA, 20 bp, synthetic construct
  misc_feature 1..20: note = human PCSK9 sgRNA54
  source 1..20: mol_type = other DNA
ggcatcccacgcagggtgag

SEQ ID NO: 94 - DNA, 20 bp, synthetic construct
  misc_feature 1..20: note = human PCSK9 sgRNA55
  source 1..20: mol_type = other DNA
gcggaaaccttctagggtgt

SEQ ID NO: 95 - DNA, 20 bp, synthetic construct
  misc_feature 1..20: note = human PCSK9 sgRNA56
  source 1..20: mol_type = other DNA
atcgtccgatggggctctgg

SEQ ID NO: 96 - protein, 544 aa, synthetic construct
  REGION 1..544: note = DNMT3A-DNMT3L(3A3L)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 97 - protein, 2111 aa, synthetic construct
  REGION 1..2111: note = DNMT3A-DNMT3L(3A3L)-dCas9-KRAB
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 98 - DNA, 20 bp, synthetic construct
  misc_feature 1..20: note = monkey PCSK9 sgRNA1
  source 1..20: mol_type = other DNA
ctgtgcacaggcttcatcat

SEQ ID NO: 99 - DNA, 20 bp, synthetic construct
  misc_feature 1..20: note = monkey PCSK9 sgRNA2
  source 1..20: mol_type = other DNA
gcacagtaacaacccctggt

SEQ ID NO: 100 - DNA, 20 bp, synthetic construct
  misc_feature 1..20: note = monkey PCSK9 sgRNA3
  source 1..20: mol_type = other DNA
ggtcccaaagagcagcagca

SEQ ID NO: 101 - DNA, 20 bp, synthetic construct
  misc_feature 1..20: note = monkey PCSK9 sgRNA4
  source 1..20: mol_type = other DNA
ctaggaggaggcctttgatg

SEQ ID NO: 102 - DNA, 20 bp, synthetic construct
  misc_feature 1..20: note = monkey PCSK9 sgRNA5
  source 1..20: mol_type = other DNA
aacaacccctggtaggtgag

SEQ ID NO: 103 - DNA, 20 bp, synthetic construct
  misc_feature 1..20: note = monkey PCSK9 sgRNA6
  source 1..20: mol_type = other DNA
ctggtaggtgagaggccaag

SEQ ID NO: 104 - DNA, 20 bp, synthetic construct
  misc_feature 1..20: note = monkey PCSK9 sgRNA7
  source 1..20: mol_type = other DNA
ctgggtgcacagtaacaacc

SEQ ID NO: 105 - DNA, 20 bp, synthetic construct
  misc_feature 1..20: note = monkey PCSK9 sgRNA8
  source 1..20: mol_type = other DNA
tccattctttctctaggagg

SEQ ID NO: 106 - DNA, 20 bp, synthetic construct
  misc_feature 1..20: note = monkey PCSK9 sgRNA9
  source 1..20: mol_type = other DNA
tgtccctctgtgcacaggct

SEQ ID NO: 107 - DNA, 20 bp, synthetic construct
  misc_feature 1..20: note = monkey PCSK9 sgRNA10
  source 1..20: mol_type = other DNA
gggtgcacagtaacaacccc

SEQ ID NO: 108 - DNA, 20 bp, synthetic construct
  misc_feature 1..20: note = monkey PCSK9 sgRNA11
  source 1..20: mol_type = other DNA
tccagttacaggcaacagga

(19) United States
(12) Patent Application Publication
(10) Pub. No.: US 2024/0408236 A1
(43) Pub. Date: Dec. 12, 2024

(54) METHOD OF MODULATING PCSK9 AND USES THEREOF

(71) Applicant: Epigenic Therapeutics, Inc., Shanghai (CN)

(72) Inventors: Changyang ZHOU, Shanghai (CN); Yidi SUN, Shanghai (CN); Wenbo PENG, Shanghai (CN); Shaoshuai MAO, Shanghai (CN)

(21) Appl. No.: 18/712,079

(22) PCT Filed: Nov. 25, 2022
(86) PCT No.: PCT/CN2022/134447

§ 371 Date: May 21, 2024

(30) Foreign Application Priority Data
Nov. 26, 2021 (WO) PCT/CN2021/133681

(51) Int. Cl. A61K 48/00; C12N 9/10; C12N 9/22; C12N 15/113; C12N 15/90

(52) U.S. Cl. CPC A61K 48/005; C12N 9/1007; C12N 9/22; C12N 15/1137; C12N 15/907; C12Y 201/01037; C07K 2319/81; C12N 2310/20

(57) ABSTRACT

This disclosure provides CRISPR/Cas9 based fusion molecules and guide RNAs for use in in vivo targeted reduction or elimination of PCSK9 gene products. This disclosure also relates to formulations, methods of production and methods of use thereof.

### RELATED APPLICATIONS

The application claims priority to, and the benefit of, PCT Application No. PCT/CN2021/133681, filed on Nov. 26, 2021, the contents of which are incorporated by reference in their entirety.

### INCORPORATION BY REFERENCE OF SEQUENCE LISTING

The Sequence Listing XML associated with this application is provided electronically in XML file format and is hereby incorporated by reference into the specification. The name of the XML file containing the Sequence Listing XML is “EPIG-001_001WO_SequenceListing_ST26”. The XML file is 220,215 bytes in size, created on Nov. 1, 2022.

### BACKGROUND

Engineered DNA-binding proteins that can be customized to target any gene in mammalian cells have enabled rapid advances in biomedical research and are a promising platform for gene therapies. The RNA-guided CRISPR-Cas9 system has emerged as a promising platform for programmable targeted gene regulation. Fusion of catalytically inactive, “dead” Cas9 (dCas9) to the Kruppel-associated box (KRAB) domain generates a synthetic repressor capable of highly specific and potent modulation or silencing of target genes in cell culture experiments.

However, persistent modulation and silencing of endogenous genes using synthetic dCas9-KRAB fusion proteins have presented challenges for use in in vivo therapies. Synthetic repressors exceed size packaging limits of viral vector delivery methods. Safety, toxicity, immunogenicity, and off target effects are other challenges that limit the use of synthetic repressors in vivo. There is a need in the art for alternative approaches for generating genetically engineered synthetic gene repressors and in vivo delivery of the synthetic gene repressors for use as therapeutics. The present disclosure addresses this unmet need in the art.

### SUMMARY

The disclosure provides a method for modulating (e.g., reducing or eliminating) the expression of a Proprotein Convertase Subtilisin/Kexin type 9 (PCSK9) gene product in a cell comprising the step of introducing into the cell: a fusion molecule comprising a least one DNA binding protein and at least one modulator of gene expression, or a nucleic acid sequence encoding the fusion molecule, wherein the modulator of gene expression provides a modification of at least one nucleotide near the PCSK9 gene and/or within a PCSK9 regulatory element, thereby modulating (e.g., reducing or eliminating) the expression of the PCSK9 gene product in the cell.

The disclosure provides an in vivo method of modulating (e.g., reducing or eliminating) the expression of a PCSK9 gene product in a subject, comprising the step of introducing to a cell of the subject: a fusion molecule comprising a least one DNA binding protein and at least one modulator of gene expression, or a nucleic acid sequence encoding the fusion molecule, wherein the modulator of gene expression provides a modification of at least one nucleotide near the PCSK9 gene and/or within a PCSK9 regulatory element, thereby modulating (e.g., reducing or eliminating) the expression of the PCSK9 gene product in the subject.

The disclosure provides a method of modulating (e.g., reducing) low density lipoprotein (LDL) cholesterol in a subject, comprising the step of introducing to a cell of the subject: a fusion molecule comprising a least one DNA binding protein and at least one modulator of gene expression, or a nucleic acid sequence encoding the fusion molecule, wherein the modulator of gene expression provides a modification of at least one nucleotide near the PCSK9 gene and/or within a PCSK9 regulatory element, thereby modulating (e.g., reducing) LDL cholesterol in the subject.

The disclosure provides a method for treating or alleviating a symptom of a PCSK9 related disorder in a subject, comprising the step of introducing to a cell of the subject: a fusion molecule comprising a least one DNA binding protein and at least one modulator of gene expression, or a nucleic acid sequence encoding the fusion molecule, wherein the modulator of gene expression provides a modification of at least one nucleotide near the PCSK9 gene and/or within a PCSK9 regulatory element, thereby treating or alleviating a symptom of a PCSK9 related disorder in the subject.

This disclosure provides a method of expanding a population of cells with a reduced expression of a PCSK9 gene product comprising the steps of: i) introducing into a plurality of cells a fusion molecule comprising a least one DNA binding protein and at least one modulator of gene expression, or a nucleic acid sequence encoding the fusion molecule, wherein the modulator of gene expression provides a modification of at least one nucleotide near the PCSK9 gene and/or within a PCSK9 regulatory element, ii) expanding the plurality of cells to produce a plurality of modified cells that have a reduced expression of the PCSK9 gene product, wherein the plurality of modified cells has at least 50%, at least 60%, at least 70%, at least 80% or at least 90% reduction in the PCSK9 gene product expression relative to a cell that has not been introduced with the fusion molecule or the nucleic acid sequence, and wherein the cell is a liver cell. In some embodiments, the reduced expression of the PCSK9 gene product is transiently reduced. In some embodiments, the reduced expression of the PCSK9 gene product is stably reduced.

In some embodiments, the PCSK9 regulatory element is a core promoter, a proximal promoter, a distal enhancer, a silencer, an insulator element, a boundary element or a locus control region.

In some embodiments, the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the PCSK9 gene. In some embodiments, the modification of at least one nucleotide near the PCSK9 gene and/or within a PCSK9 regulatory element is located within 1000 bp upstream of the transcription start site of the PCSK9 gene. In some embodiments, the modification of at least one nucleotide near the PCSK9 gene and/or within a PCSK9 regulatory element is located within 300 bp upstream of the transcription start site of the PCSK9 gene.

In some embodiments, the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the PCSK9 gene. In some embodiments, the modification of at least one nucleotide near the PCSK9 gene and/or within a PCSK9 regulatory element is located within about 300 bp downstream of the transcription start site of the PCSK9 gene. In some embodiments, the modification of at least one nucleotide near the PCSK9 gene and/or within a PCSK9 regulatory element is located within 1000 bp upstream of the transcription start site and within 300 bp downstream of the transcription start site of the PCSK9 gene.

In some embodiments, the modification of at least one nucleotide is a DNA methylation.

In some embodiments, the at least one modulator of gene expression comprises a DNA methyltransferase (DNMT), a DNA demethylase, a histone methyltransferase, a histone demethylase, or a portion thereof.

In some embodiments, the at least one modulator of gene expression comprises a DNA methyltransferase (DNMT) or a portion thereof. In some embodiments, the DNA methyltransferase is DNMT3A, DNMT3B, DNMT3L, DNMT1 or DNMT2. In some embodiments, the DNMT3A comprises the amino acid sequence of SEQ ID NO: 23. In some embodiments, the DNMT3L comprises the amino acid sequence of SEQ ID NO: 24.

In some embodiments, the at least one modulator of gene expression comprises a zinc finger protein-based transcription factor or a portion thereof. In some embodiments, the zinc finger protein-based transcription factor is Kruppel-associated suppression box (KRAB). In some embodiments, the KRAB comprises the amino acid sequence of SEQ ID NO: 22.

In some embodiments, the at least one modulator of gene expression comprises a DNA methyltransferase or a portion thereof and a zinc finger protein-based transcription factor or a portion thereof. In some embodiments, the DNA methyltransferase is selected from DNMT3A and DNMT3L and a combination thereof, and the zinc finger protein-based transcription factor is KRAB.

In some embodiments, the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease. In some embodiments, the at least one DNA binding protein is dCas9. In some embodiments, the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, or a Streptococcus thermophilus (e.g., strain LMD-9) dCas9. In some embodiments, the dCas9 comprises the amino acid sequence of SEQ ID NO: 1.

In some embodiments, the fusion molecule comprises the at least one modulator of gene expression fused to the C-terminus, the N-terminus, or both, of the at least one DNA binding protein.

In some embodiments, the at least one modulator of gene expression is fused directly to the at least one DNA binding protein. In some embodiments, the at least one modulator of gene expression is fused indirectly with the at least one DNA binding protein via a non-modulator, a second modulator, or a linker. In some embodiments, the fusion molecule comprises a dCas9 fused with a KRAB on the C-terminal end and a DNMT3A and a DNMT3L on the N-terminal end. In some embodiments, the fusion molecule comprises the amino acid sequence of SEQ ID NO: 97.

In some embodiments, the fusion molecule further comprises at least one nuclear localization sequence. In some embodiments, the at least one nuclear localization sequence is directly fused to the C-terminus, the N-terminus or both of the at least one DNA binding protein. In some embodiments, the at least one nuclear localization sequence is indirectly fused to the C-terminus, the N-terminus or both of the at least one DNA binding protein via a linker.

In some embodiments, the nucleic acid sequence encoding the fusion molecule is a deoxyribonucleic acid (DNA). In some embodiments, the nucleic acid sequence encoding the fusion molecule is a messenger ribonucleic acid (mRNA).

In some embodiments, the method further comprises the step of introducing at least one single guide RNA (sgRNA) that is complementary to a DNA sequence near the PCSK9 gene and/or within a PCSK9 regulatory element, thereby targeting the fusion molecule to the PCSK9 gene or PCSK9 regulatory element, or a DNA encoding the sgRNA. In some embodiments, the sgRNA comprises the nucleic acid sequence of SEQ ID NOS: 27-95 or 98-108.

In some embodiments, the fusion molecule is formulated in a liposome or a lipid nanoparticle. In some embodiments, the fusion molecule and the sgRNA are formulated in a liposome or a lipid nanoparticle. In some embodiments, the fusion molecule and the sgRNA are formulated in the same liposome or lipid nanoparticle. In some embodiments, the fusion molecule and the sgRNA are formulated in different liposome or lipid nanoparticle.

In some embodiments, the liposome or lipid nanoparticle comprises of ionizable lipids (20%-70%, molar ratio), PEGylated lipids (0%-30%, molar ratio), supporting lipids (5%-50%, molar ratio), and cholesterol (10%-50%, molar ratio). In some embodiments, the ionizable lipid is selected from a group consisting of pH-responsive ionizable lipids, thermal-responsive ionizable lipids and light-responsive ionizable lipids.

In some embodiments, the fusion molecule is formulated in an AAV vector. In some embodiments, the fusion molecule and the sgRNA are formulated in an AAV vector. In some embodiments, the fusion molecule and the sgRNA are formulated in the same AAV vector. In some embodiments, the fusion molecule and the sgRNA are formulated in different AAV vectors.

In some embodiments, the fusion molecule is delivered to the cell by local injection, systemic infusion, or a combination thereof.

In some embodiments, the subject is a human.

In some embodiments, the PCSK9 related disorder is a high atherosclerotic cardiovascular disease. In some embodiments, the PCSK9 related disorder is hypercholesterolemia. In some embodiments, the cell is a hepatocyte.

The disclosure provides a sgRNA comprising the nucleic acid sequence of any one of SEQ ID NOs: 27-95 or 98-108. The disclosure provides a DNA sequence encoding any one of the sgRNA disclosed herein.

The disclosure provides a pharmaceutical composition comprising a fusion molecule comprising a least one DNA binding protein and at least one modulator of gene expression, or a nucleic acid sequence encoding the fusion molecule, wherein the fusion molecule is targeted to a genomic region near a PCSK9 gene and/or within a PCSK9 regulatory element, wherein the at least one modulator of gene expression provides a modification of at least one nucleotide near the PCSK9 gene and/or within a PCSK9 regulatory element, wherein the at least one modulator of gene expression comprises a DNA methyltransferase (DNMT), a DNA demethylase, a histone methyltransferase, a histone demethylase, or a portion thereof, or a zinc finger protein-based transcription factor or a portion thereof, or a combination thereof, and wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

In some embodiments, the PCSK9 regulatory element is a transcription start site, core promoter, a proximal promoter, a distal enhancer, a silencer, an insulator element, a boundary element or a locus control region.

In some embodiments, the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the PCSK9 gene. In some embodiments, the modification of at least one nucleotide near the PCSK9 gene and/or within a PCSK9 regulatory element is located within 1000 bp upstream of the transcription start site of the PCSK9 gene. In some embodiments, the modification of at least one nucleotide near the PCSK9 gene and/or within a PCSK9 regulatory element is located within 300 bp upstream of the transcription start site of the PCSK9 gene.

In some embodiments, the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the PCSK9 gene. In some embodiments, the modification of at least one nucleotide near the PCSK9 gene and/or within a PCSK9 regulatory element is located within about 300 bp downstream of the transcription start site of the PCSK9 gene. In some embodiments, the modification of at least one nucleotide near the PCSK9 gene and/or within a PCSK9 regulatory element is located within 1000 bp upstream of the transcription start site and within 300 bp downstream of the transcription start site of the PCSK9 gene.

In some embodiments, the modification of at least one nucleotide is a DNA methylation. In some embodiments, the at least one modulator of gene expression comprises a DNA methyltransferase (DNMT) or a portion thereof. In some embodiments, the DNA methyltransferase is DNMT3A, DNMT3B, DNMT3L, DNMT1 or DNMT2. In some embodiments, the DNMT3A comprises the amino acid sequence of SEQ ID NO: 23. In some embodiments, the DNMT3L comprises the amino acid sequence of SEQ ID NO: 24.

In some embodiments, the at least one modulator of gene expression comprises a zinc-finger protein-based transcription factor or a portion thereof. In some embodiments, the zinc finger protein-based transcription factor is Kruppel-associated suppression box (KRAB). In some embodiments, the KRAB comprises the amino acid sequence of SEQ ID NO: 22.

In some embodiments, the at least one modulator of gene expression comprises a DNA methyltransferase or a portion thereof and a zinc finger protein-based transcription factor or a portion thereof. In some embodiments, the DNA methyltransferase is selected from DNMT3A and DNMT3L and a combination thereof, and the zinc finger protein-based transcription factor is KRAB. In some embodiments, the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

In some embodiments, the at least one DNA binding protein is dCas9. In some embodiments, the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, or a Streptococcus thermophilus (e.g., strain LMD-9) dCas9. In some embodiments, the dCas9 comprises the amino acid sequence of SEQ ID NO: 1.

In some embodiments, the fusion molecule comprises the at least one modulator of gene expression fused to the C-terminus, the N-terminus, or both, of the at least one DNA binding protein.

In some embodiments, the at least one modulator of gene expression is fused directly to the at least one DNA binding protein. In some embodiments, the at least one modulator of gene expression is fused indirectly with the at least one DNA binding protein via a non-modulator, a second modulator, or a linker.

In some embodiments, the fusion molecule comprises a dCas9 fused with a KRAB on the C-terminal end and a DNMT3A and a DNMT3L on the N-terminal end. In some embodiments, the fusion molecule comprises the amino acid sequence of SEQ ID NO: 97.

In some embodiments, the fusion molecule further comprises at least one nuclear localization sequence. In some embodiments, the at least one nuclear localization sequence is directly fused to the C-terminus, the N-terminus or both of the at least one DNA binding protein. In some embodiments, the at least one nuclear localization sequence is indirectly fused to the C-terminus, the N-terminus or both of the at least one DNA binding protein via a linker.

In some embodiments, the nucleic acid sequence encoding the fusion molecule is a deoxyribonucleic acid (DNA). In some embodiments, the nucleic acid sequence encoding the fusion molecule is a messenger ribonucleic acid (mRNA).

In some embodiments, the pharmaceutical composition further comprises at least one single guide RNA (sgRNA) that is complementary to a DNA sequence near the PCSK9 gene and/or within a PCSK9 regulatory element. In some embodiments, the sgRNA comprises the nucleic acid sequence of SEQ ID NOS: 27-95 or 98-108.

In some embodiments, the fusion molecule is packaged in a liposome or a lipid nanoparticle. In some embodiments, the fusion molecule and the sgRNA are packaged in a liposome or a lipid nanoparticle. In some embodiments, the sgRNA are packaged in the same liposome or lipid nanoparticle. In some embodiments, the fusion molecule and the sgRNA are packaged in a different liposome or lipid nanoparticle.

In some embodiments, the liposome or the lipid nanoparticle comprises of ionizable lipids (20%-70%, molar ratio), PEGylated lipids (0%-30%, molar ratio), supporting lipids (5%-50%, molar ratio), and cholesterol (10%-50%, molar ratio). In some embodiments, the ionizable lipid is selected from a group consisting of pH-responsive ionizable lipids, thermal-responsive ionizable lipids and light-responsive ionizable lipids.

In some embodiments, the fusion molecule is packaged in an AAV vector. In some embodiments, the fusion molecule and the sgRNA are packaged in an AAV vector. In some embodiments, the fusion molecule and the sgRNA are packaged in the same AAV vector.

In some embodiments, the fusion molecule and the sgRNA are packaged in different AAV vectors.

### BRIEF DESCRIPTION OF THE DRAWINGS

FIG. 1A is a schematic diagram showing the “EPICAS” (also referred to as “CRISPRoff”) dual plasmid system and a sgRNA tiling screen design targeting mouse PCSK9 expression. The first plasmid (“catalytic protein” plasmid or “fusion molecule” plasmid), encodes DNMT3A-DNMT3L(3A3L)-dCas9-KRAB, under the control of a CAG promoter, and a GFP marker separated by 2A elements. The second plasmid (“sgRNA” plasmid) has an sgRNA-scaffold under the control of a U6 promoter and a mCherry marker under the control of a CMV promoter. The sgRNAs of the tiling screen target the transcription start site (TSS)+250 bp upstream of the mouse PCSK9 protein coding sequence (CDS).

FIG. 1B is a bar graph showing relative mRNA expression following transfection of a mouse AML12 cell line with the catalytic protein plasmid and a single PCSK9 sgRNA plasmid. n=3 biological replicates for each group.

FIG. 1C is a bar graph showing the relative mRNA expression following transfection of a mouse AML12 cell line with the catalytic protein plasmid and a mixture of various PCSK9 sgRNA plasmids.

FIG. 1D is a bar graph showing the relative mRNA expression following transfection of a mouse Ai9 primary hepatocytes with the catalytic protein plasmid and a mixture of various PCSK9 sgRNA plasmids.

FIG. 2A is a schematic diagram showing the EPICAS mRNA plasmid design. The EPICAS ORF comprises a DNMT3A-DNMT3L-dCas9-KRAB cassette. The plasmid can be digested at the XbaI and BpiI restriction sites to form a linearized plasmid.

FIG. 2B is an electrophoretogram of purified mRNA expressed from the EPICAS mRNA plasmid.

FIG. 2C is a schematic diagram showing the Snrpn-GFP reporter system.

FIG. 2D are a series of flow cytometry graphs showing Snrpn-GFP expression 8 days post-transfection with either an Snrpn sgRNA or a non-targeting sgRNA, or 30 days, 150 days, and 400 days post-transfection with the Snrpn sgRNA.

FIG. 2E is a line graph showing the percent of cells with GFP-off, 8 days post-transfection with either an Snrpn sgRNA or a non-targeting sgRNA (NT-sgRNA), or 30 days, 150 days, and 400 days post-transfection with the Snrpn sgRNA.

FIG. 2F is a schematic diagram of DNA methylation levels of the Snrpn locus using a bisulfite PCR analysis. Bisulfite sequencing analysis of the Snrpn locus between CRISPRoff targeting and non-targeting cells was shown 8 days after transfection. Each row represents one single clone and sequencing read, and each column indicates one specific genomic position. White dots represent unmethylated CpG dinucleotides and black dots represent methylated CpG dinucleotides. The bottom graph represents the methylation at 13 positions of the Snrpn locus.

FIG. 2G is a schematic representation of a bisulfite sequencing analysis of the Snrpn locus between CRISPRoff targeting an non-targeting cells at 400 days post transfection. The back areas of the circles represents the average degree (n=9 sequencing reads) of methylation for each CpG dinucleotide.

FIG. 2H is a capillary gel electrophoresis showing the purity of CRISPRoff mRNA by in vitro transcription.

FIG. 2I is a series of graphs showing the percent of GFP-off cells at 70 and 90 days after CRISPRoff transfection.

FIG. 3A is a schematic diagram of a lipid nanoparticle (LNP) design. Epigenetic CRISPR/Cas elements and sgRNA elements may be encapsulated by LNPs.

FIG. 3B is a transmission electron microscope image showing LNPs containing EPICAS.

FIG. 3C is a graph showing the size distribution of LNPs.

FIG. 3D are a series of pictures showing in vivo fluorescence imaging of luciferase mRNAs delivered by lipid nanoparticle to mouse liver cells by intramuscular injection.

FIG. 3E is a schematic diagram of an in vivo experimental design for delivery of LNPs containing EPICAS mRNA and mouse PCSK9 targeting sgRNA. LNPs were administered to the C57CB/6J mice via injection into the lateral tail vein and PCSK9 gene expression was analyzed 5 days post injection.

FIG. 3F is a bar graph showing relative mRNA expression of mouse PCSK9 following injection with PBS or LNP containing EPICAS mRNA and mouse PCSK9 targeting sgRNA.

FIG. 4A is a schematic diagram of an sgRNA tiling screen design for monkey PCSK9. The sgRNAs of the tiling screen target the transcription start site (TSS)+250 bp upstream of the monkey PCSK9 protein coding sequence (CDS).

FIG. 4B is a bar graph showing relative mRNA expression following transfection of a monkey cells with the catalytic protein plasmid and a single PCSK9 sgRNA plasmid.

FIG. 5A is schematic diagram showing the experimental design of an sgRNA tiling screen targeting human PCSK9.

FIG. 5B are a series of bar graphs showing mean fluorescence intensity rate of PCSK9 following transfection of human PCSK9 reporter cell lines with various sgRNAs targeting the region 300 bp upstream and 300 bp downstream of human PCSK9 TSS. The graph on the left shows results 72 hours post-transfection. The graph on the right shows results 120 hours post-transfection.

FIG. 5C is a series of graphs showing mean fluorescence intensity rate of PCSK9, 72 hours post-transfection of human PCSK9 reporter cell lines with various sgRNAs targeting the region 300 bp upstream and 300 bp downstream of human PCSK9 TSS.

FIG. 5D is a series of graphs showing the PCSK9 mRNA expression following transfection with EPICAS dual plasmid system using various sgRNAs targeting PCSK9. The graph on the left shows PCSK9 mRNA expression in Hep3B cell lines that endogenously express human PCSK9, 48 hours post-transfection with various sgRNAs. The graph on the right shows mean fluorescence intensity rate of PCSK9, 120 hours post-transfection of human PCSK9 reporter cell lines with various sgRNAs.

FIG. 6A is an image showing fluorescent staining of a mouse liver. tdTomato staining (CRISPRoff mRNA) and DAPI staining are shown. The large proportion of cells exhibiting tdTomato fluorescence shows the efficacy of LNP-mediated CRISPRoff mRNAs in mouse liver.

FIG. 6B is an image showing mouse organs after LNP-mediated CRISPRoff mRNA delivery. Luciferase imaging shows that the CRISPRoff mRNA is localized to the liver.

FIG. 6C is a series of images showing in vivo distribution of Luc mRNA-LNPs by luciferase imaging at 6 h, 12 h, 24 h and 48 h after administration.

FIG. 6D is a graph showing Pcsk9 mRNA levels in wild-type mouse liver. Relative mRNA expression levels were assessed one week after treatment with indicated doses (mg RNA per kg body weight) of LNP formulation with CRISPRoff mRNA and sgRNAs targeting Pcsk9. n=5 for PBS, and n=6 for LNP formulation groups. mg/kg (MPK).

FIG. 6E is a graph showing protein levels of Pcsk9 in the blood of mice treated with indicated doses (mg RNA per kg body weight) of LNP formulation with CRISPRoff mRNA and sgRNAs targeting Pcsk9. n=5 for PBS, and n=6 for LNP formulation groups.

FIG. 6F is a graph showing protein levels of Pcsk9 in the blood of mice at 2, 4, 6 and 8 weeks after treatment of 3 mg/kg LNP formulation with CRISPRoff mRNA and sgRNAs targeting Pcsk9. n=4 for each group.

FIG. 6G is a schematic diagram of a partial hepatectomy (PHx) and liver regeneration experiment.

FIG. 6H is a graph showing a comparison of liver Pcsk9 mRNA levels in mice at 7 days after PHx or Sham surgery.

FIG. 6I is a graph showing targeted bisulfite sequencing analysis of the CpG dinucleotides at promoter of the Pcsk9 gene. The methylation levels at each CpG dinucleotide were quantified by the averaged beta value from 4 or 3 biological replicates. n=4, 3, and 4 for PBS, Sham surgery and PHx. PHx, partial hepatectomy. P values were calculated by student's t-tests. * P<0.05, ** P<0.01, ***P<0.001, **** P<0.0001.

FIG. 7A is a schematic diagram of Pcsk9 repression by epigenome editing in mice on High Fat Diet (HFD).

FIG. 7B is a graph showing relative protein levels of Pcsk9 in the blood of mice on HFD at 7 days after treatment with PBS, 4 mg/kg or 6 mg/kg of LNP formulation with CRISPRoff mRNA and sgRNAs targeting Pcsk9. mg/kg (MPK).

FIG. 7C is a graph showing relative protein levels of Pcsk9 in the blood of mice on HFD at 14 days after treatment.

FIG. 7D is a graph showing a comparison of plasma LDL-C levels in mice on HFD before and after treatment. n=5 biological replicates for each group. P values were calculated by student's t-tests. * P<0.05, ** P<0.01, ***P<0.001, **** P<0.0001.

FIG. 8A is a scatter plot of log 2-transformed TPM for genes in two groups. The point marked corresponds with Pcsk9 gene. The data represent the mean of three independent biological replicates.

FIG. 8B is a graphical depiction of gene expression changes within 1 Mb upstream and downstream from the targeted gene Pcsk9. The x-axis represents the genomic position of each gene.

FIG. 8C is a Manhattan plot showing differentially methylated CpGs on the whole genome between CRISPRoff- and PBS-treated mice. The points above −log 10 (p-value) of 0 indicate that the methylation level of the locus is higher than in CRISPRoff-treated mice, and the points below −log 10 (p-value) of 0 indicate the methylation level is higher in the PBS-treated mice. The arrow marks the genomic position of Pcsk9.

FIG. 8D is a graphical depiction of methylation changes within 1 kb upstream and downstream from Pcsk9. The x-axis represents the genomic position. The y-axis represents −log (p-value) Red dots indicate within the range of Pcsk9 gene from transcriptional start site to the end of its stop codon, and blue dots indicate outside of this range. The protospacer sites targeted by the two sgRNAs are labelled as “sgRNA7” and “sgRNA9”.

FIG. 8E is a Volcano plot showing gene expression changes for predicted sgRNA-dependent off-target genes and the target gene. The x-axis represents the log 2 transformed fold changes of indicated genes obtained by DESeq2 and the y-axis represents the log 10 transformed adjusted P values. n=3 biological replicates for each group.

FIG. 9A is a graph showing the size distribution of LNPs encapsulating CRISPRoff and sgRNAs targeting Pcsk9.

FIG. 9B is a Cryo-EM image of LNPs encapsulating CRISPRoff and sgRNAs targeting Pcsk9.

FIG. 9C is a graph showing targeted bisulfite sequencing analysis of the CpG dinucleotides on Pcsk9 gene treated with different doses of LNP encapsulated CRISPRoff. The methylation levels at each CpG dinucleotide were quantified by the averaged beta value from 4 or 3 biological replicates. n=3 for each group. mg/kg (MPK).

FIG. 9D is a series of graphs showing absolute values of blood levels of aspartate transaminase (ALT), alanine transaminase (AST), alkaline phosphatase (ALP) and Albumin (ALB) in mice before, 4 and 7 days after treatment of 3 or 6 mg/kg LNP encapsulated CRISPRoff. n=6 for each group. P values were calculated by student's t-tests. * P<0.05, ** P<0.01, ***P 0.001, **** P<0.0001.

FIG. 10A is a volcano plot showing gene expression changes for whole transcriptomic genes. The point representing Pcsk9 is indicated.

FIG. 10B is a graph showing a comparison of methylation levels within a 50 kb genomic window, flanking the Pcsk9 gene. Bars above the x-axis represent loci with methylation levels greater than 0.5 and bars below the x-axis represent methylation levels less than 0.5. The protospacer sites targeted by the two sgRNAs are labelled as “sgRNA7” and “sgRNA9”.

FIG. 10C is a bar graph showing log 2-tansformed fold changes of methylation and gene expression levels of indicated genes showing statistically significance in gene expression levels between CRISPRoff- and PBS-treated mice.

FIG. 10D is a bar graph showing log 2-tansformed fold changes of methylation and gene expression levels of indicated genes showing statistically significance in methylation levels between CRISPRoff- and PBS-treated mice. n=3 biological replicates for each group.

### DETAILED DESCRIPTION

The present disclosure overcomes problems associated with current technologies by providing genetically engineered fusion molecules (e.g., DNMT3A-DNMT3L(3A3L)-dCas9-KRAB fusion molecule) for targeted reduction or elimination of gene products (e.g., PCSK9) in a cell for use in in vivo gene therapy. The genetically engineered fusion molecules of the disclosure are useful for treatment of genetic diseases, including for example, diseases of the liver, diseases associated with high cholesterol, and diseases associated with dysregulation of cholesterol (e.g. low density lipoprotein (LDL) cholesterol). Accordingly, methods of making genetically engineered fusion molecules and pharmaceutical formulations thereof (e.g., lipid nanoparticle formulations) for use in in vivo delivery are also provided.

### I. Definitions

As used herein, the term “coding sequence” or “encoding nucleic acid” means the nucleicacids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The term “complement” or “complementary” as used herein with reference to a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The term “correcting”, “genome editing” and “restoring” refers to changing a mutant gene that encodes a mutant protein, a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

As used herein, the term “donor DNA”, “donor template” and “repair template” refers to a double-stranded DNA fragment or molecule that includes at least a portion of the gene of interest. The donor DNA may encode a full-functional protein or a partially-functional protein.

As used herein, the terms “frameshift” or “frameshift mutation” are used interchangeably and refer to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

As used herein, the term “functional” and “full-functional” describes a protein that has biological activity. A “functional gene” refers to a gene transcribed to mRNA, which is translated to a functional protein.

As used herein, the term “fusion protein” refers to a chimeric protein created through the covalent or non-covalent joining of two or more genes, directly or indirectly, that originally coded for separate proteins. In some embodiments, the translation of the fusion gene results in a single polypeptide with functional properties derived from each of the original proteins.

As used herein, the term “genetic construct” refers to the DNA or RNA molecules that comprise a nucleotide sequence that encodes a protein. The coding sequence includes initiation and termination signals operably linked to regulatory elements including a promoter and polyadenylation signal capable of directing expression in cells.

The term “Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, nonhomologous end joining may take place instead.

The term “genome editing” as used herein refers to changing a gene. Genome editing may include correcting or restoring a mutant gene. Genome editing may include knocking out a gene, such as a mutant gene or a normal gene. Genome editing may be used to treat disease by changing the gene of interest.

The term “identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the terms “mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

As used herein, the term “modulator of epigenetic modification” refers to an agent that targets gene expression via epigenetic modification (e.g., via histone acetylation or methylation, or DNA methylation at a regulatory element of target gene, e.g., a promoter, enhancer or transcription start site). Chromatin remodeling and DNA methylation are two main mechanisms for regulating gene transcription. Specific epigenetic marks (e.g., DNA methylation) structurally or biochemically direct gene transcription or gene silencing/repression. For example, DNA methylation of regions that regulate transcriptional activities alter gene expression without changing the underlying DNA sequence. Transcriptional regulation using epigenetic modification (e.g., DNA methylation) allows for targeted modulation of gene expression, without affecting the expression of other gene products.

The term “non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

The term “normal gene” as used herein refers to a gene that has not undergone a change, such as a loss, gain, or exchange of genetic material. The normal gene undergoes normal gene transmission and gene expression.

The term “nuclease mediated NHEJ” as used herein refers to NHEJ that is initiated after a nuclease, such as a cas9, cuts double stranded DNA.

As used herein, the term “nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, the term “operably linked” means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “partially-functional” as used herein describes a protein that is encoded by a mutant gene and has less biological activity than a functional protein but more than a non-functional protein. In one embodiment, a partially-functional protein shows a biological activity that is less than 95%, 90%, 85%, 80%, 75%, 70%, 65%, 60%, 55%, 50%, 45%, 40%, 35%, or 30% of that of a corresponding functional protein.

The term “premature stop codon” or “out-of-frame stop codon” as used interchangeably herein refers to nonsense mutation in a sequence of DNA, which results in a stop codon at a location not normally found in the wild-type gene. A premature stop codon may cause a protein to be truncated or shorter compared to the full-length version of the protein.

The term “promoter” or “core promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of a nucleic acid. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

The term “target gene” as used herein refers to any nucleotide sequence encoding a known or putative gene product. The target gene may be a mutated gene involved in a genetic disease or disorder.

The term “target region” as used herein refers to the region of the target gene to which the site-specific nuclease is designed to bind.

As used herein, the term “transgene” refers to a gene or genetic material containing a gene sequence that has been isolated from one organism and is introduced into a different organism. Alternatively, the term “transgene” also refers to a gene or genetic material that is chemically synthesized and introduced into an organism. This non-native segment of DNA may retain the ability to produce RNA or protein in the transgenic organism, or it may alter the normal function of the transgenic organism's genetic code. The introduction of a transgene has the potential to change the phenotype of an organism.

As used herein, the term “variant” when used with respect to a nucleic acid means (i) a portion or fragment of a referenced nucleotide sequence; (ii) the complement of a referenced nucleotide sequence or portion thereof, (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity.

Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157: 105-132(1982), incorporated herein by reference in its entirety. The hydropathic index of an amino acid is based on a consideration of its hydrophobicity and charge. It is known in the art that amino acids of similar hydropathic indexes may be substituted and still retain protein function. In one aspect, amino acids having hydropathic indexes of 2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, the term “vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, such as a DNA plasmid.

As used herein, the terms “gene transfer,” “gene delivery,” and “gene transduction” refer to methods or systems for reliably inserting a particular nucleotide sequence (e.g., DNA or RNA), fusion protein, polypeptide and the like into targeted cells.

As used herein, the terms “adenoviral associated virus (AAV) vector,” “AAV gene therapy vector,” and “gene therapy vector” refer to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, and AAV-6. The ITRs, however, need not be the wild-type nucleotide sequences, and may be altered (e.g., by the insertion, deletion or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (e.g., functional ITRs) of the virus.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

The “transgene” may contain a transgenic sequence or a native or wild-type DNA sequence. The transgene may become part of the genome of the subject. A transgenic sequence can be partly or entirely species-heterologous, i.e., the transgenic sequence, or a portion thereof, can be from a species which is different from the cell into which it is introduced.

As used herein, the term “stably maintained” refers to characteristics of transgenic subject (e.g., a human or non-human primate) that maintain at least one of their transgenic elements (i.e., the element that is desired) through multiple generations of cells. For example, it is intended that the term encompass many cell division cycles of the originally transfected cell. The term “stable transfection” or “stably transfected” refers to the introduction and integration of foreign DNA into the genome of the cell. The term “stable transfectant” refers to a cell that has stably integrated foreign DNA into the genomic DNA.

As used herein, the terms “transgene encoding,” “nucleic acid molecule encoding,” “DNA sequence encoding,” and “DNA encoding” refer to the order or sequence of deoxyribonucleotides along a strand of deoxyribonucleic acid. The order of these deoxyribonucleotides may, for example, determine the order of amino acids along the polypeptide (protein) chain. The DNA sequence thus may code for the amino acid sequence.

As used herein, the term “wild type” (wt) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants may be isolated, which are identified by the acquisition of altered characteristics when compared to the wild-type gene or gene product.

As used herein, the term “transfection” refers to the uptake of a foreign nucleic acid (e.g., DNA or RNA) by a cell. A cell has been “transfected” when an exogenous nucleic acid (DNA or RNA) has been introduced inside the cell membrane. A number of transfection techniques are generally known in the art (See, e.g., Graham et al., Virol., 52:456 (1973); Sambrook et al., Molecular Cloning, a Laboratory Manual, Cold Spring Harbor Laboratories, New York (1989); Davis et al., Basic Methods in Molecular Biology, Elsevier, (1986); and Chu et al., Gene 13:197 (1981), incorporated herein by reference in their entirety). Such techniques may be used to introduce one or more exogenous DNA moieties, such as a gene transfer vector and other nucleic acid molecules, into suitable recipient cells.

As used herein, the terms “stable transfection” and “stably transfected” refers to the introduction and integration of foreign DNA into the genome of the transfected cell. The term “stable transfectant” refers to a cell, which has stably integrated foreign DNA into the genomic DNA.

As used herein, the term “transient transfection” or “transiently transfected” refers to the introduction of foreign DNA into a cell wherein the foreign DNA fails to integrate into the genome of the transfected cell and is maintained as an episome. During this time the foreign DNA is subject to the regulatory controls that govern the expression of endogenous genes in the chromosomes. The term “transient transfectant” refers to cells which have taken up foreign DNA but have failed to integrate this DNA. As used herein, the term “transduction” denotes the delivery of a DNA molecule to a recipient cell either in vivo or in vitro, via a replication-defective viral vector, such as via a recombinant AAV virion.

As used herein, the term “recipient cell” refers to a cell which has been transfected or transduced, or is capable of being transfected or transduced, by a nucleic acid construct or vector bearing a selected nucleotide sequence of interest. The term includes the progeny of the parent cell, whether or not the progeny are identical in morphology or in genetic make-up to the original parent, so long as the selected nucleotide sequence is present. The recipient cell may be the cells of a subject to which the gene therapy particles and/or gene therapy vector has been administered.

As used herein, the term “recombinant DNA molecule” refers to a DNA molecule which is comprised of segments of DNA joined together by means of molecular biological techniques.

As used herein, the term “regulatory element” refers to a genetic element which can control the expression of nucleic acid sequences. For example, a promoter is a regulatory element that facilitates the initiation of transcription of an operably linked coding region. Other regulatory elements are splicing signals, polyadenylation signals, termination signals, etc.

The term DNA “control sequences” refers collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need be present.

The term “enhancer” as used herein refers to non-coding DNA sequences containing multiple activator and repressor binding sites. Enhancers range from 50 bp to 1500 bp in length and may be either proximal, 5′ upstream to the promoter, within any intron of the regulated gene, or distal, in introns of neighboring genes, or intergenic regions far away from the locus, or on regions on different chromosomes. More than one enhancer may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements. An enhancer and a promoter used can be “endogenous,” “exogenous,” or “heterologous” with respect to the gene to which they are operably linked. An “endogenous” enhancer/promoter is one which is naturally linked with a given gene in the genome. An “exogenous” or “heterologous” enhancer or promoter is one which is placed in juxtaposition to a gene by means of genetic manipulation (i.e., molecular biological techniques) such that transcription of that gene is directed by the linked enhancer/promoter.

The term “insulators” or “insulator element” as used herein refers to a genetic boundary element that blocks the interaction between enhancers and promoters. By residing between the enhancer and promoter, the insulator may inhibit their subsequent interactions. Insulators can determine the set of genes an enhancer can influence. Insulators are needed where two adjacent genes on a chromosome have very different transcription patterns and the inducing or repressing mechanisms of one does not interfere with the neighboring gene. Insulators have also been found to cluster at the boundaries of topological association domains (TADs) and may have a role in partitioning the genome into “chromosome neighborhoods”—genomic regions within which regulation occurs. Insulator activity is thought to occur primarily through the 3D structure of DNA mediated by proteins including CTCF. Insulators are likely to function through multiple mechanisms. Many enhancers form DNA loops that put them in close physical proximity to promoter regions during transcriptional activation. Insulators may promote the formation of DNA loops that prevent the promoter-enhancer loops from forming. Barrier insulators may prevent the spread of heterochromatin from a silenced gene to an actively transcribed gene.

The term “locus control regions” as used herein refers to a long-range cis-regulatory element that enhances expression of linked genes at distal chromatin sites. It functions in a copy number-dependent manner and is tissue-specific, as seen in the selective expression of 3-globin genes in erythroid cells. Expression levels of genes can be modified by the LCR and gene-proximal elements, such as promoters, enhancers, and silencers. The LCR functions by recruiting chromatin-modifying, coactivator, and transcription complexes. Its sequence is conserved in many vertebrates, and conservation of specific sites may suggest importance in function.

The term “silencers” or “repressors” as used interchangeably herein refer to a DNA sequence capable of binding transcription regulation factors and preventing genes from being expressed as proteins. A silencer is a sequence-specific element that induces a negative effect on the transcription of its particular gene. There are many positions in which a silencer element can be located in DNA. The most common position is found upstream of the target gene where it can help repress the transcription of the gene. This distance can vary greatly between approximately −20 bp to −2000 bp upstream of a gene. Certain silencers can be found downstream of a promoter located within the intron or exon of the gene itself. Silencers have also been found within the 3 prime untranslated region (3′ UTR) of mRNA. There are two main types of silencers in DNA, which are the classical silencer element and the non-classical negative regulatory element (NRE). In classical silencers, the gene is actively repressed by the silencer element, mostly by interfering with general transcription factor (GTF) assembly. NREs passively repress the gene, usually by inhibiting other elements that are upstream of the gene.

As used herein, the term “tissue specific” refers to regulatory elements or control sequences, such as a promoter, an enhancer, etc., wherein the expression of the nucleic acid sequence is substantially greater in a specific cell type(s) or tissue(s).

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York (1989), pp. 16.7-16.8, incorporated herein by reference in its entirety). A commonly used splice donor and acceptor site is the splice junction from the 16S RNA of SV40.

Transcription termination signals are generally found downstream of a polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which has been isolated from one gene and operably linked to the 3′ end of another gene. A commonly used heterologous poly A signal is the SV40 poly A signal. The SV40 poly A signal is contained on a 237 bp BamHI/BclI restriction fragment and directs both termination and polyadenylation (Sambrook et al., supra, at 16.6-16.7, incorporated herein by reference in its entirety).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

As defined herein, a “therapeutically effective amount” or “therapeutic effective dose” is an amount or dose of a fusion protein, polypeptide, nucleic acid, lipid nanoparticle, liposome, AAV particle(s), or virion(s) capable of producing sufficient amounts of a desired protein to modulate the activity of the protein in a desired manner, thus providing a palliative tool for clinical intervention. In some embodiments, a therapeutically effective amount or dose of a transfected fusion protein, polypeptide, nucleic acid, AAV particle(s), or virion(s) as described herein is enough to confer suppression of a gene targeted by the fusion protein/gene therapy construct.

As used herein, the term “treat”, e.g., a disorder, means that a subject (e.g., a human) who has a disorder, is at risk of having a disorder, and/or experiences a symptom of a disorder, will, in an embodiment, suffer a less severe symptom and/or will recover faster, when a fusion molecule or a nucleic acid that encodes the fusion molecule, and/or a gRNA or a nucleic acid that encodes the gRNA, e.g., as described herein, is administered than if the fusion molecule or a nucleic acid that encodes the fusion molecule, and/or the gRNA or a nucleic acid that encodes the gRNA, were never administered.

### II. DNA Binding Proteins

In certain embodiments in the methods and compositions as defined herein according to the disclosure, the DNA binding protein (e.g. DNA targeting agent) comprises a (DNA) nuclease, such as a nuclease which can target DNA in a sequence specific manner or which can be directed or instructed to target DNA in a sequence specific manner, such as a CRISPR-Cas system, Zinc finger nuclease (ZFN), Transcription Activator-Like Effector Nuclease (TALEN), or meganuclease. In some embodiments, the DNA binding protein is a DNA nuclease derived from a CRISPR-Cas system.

### Transcription Activator-Like Effector Nuclease (TALEN) System

In certain embodiments, the nucleic acid binding protein (e.g., DNA binding protein) is a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29: 149-153 and U.S. Pat. Nos. 8,450,471, 8,440,431 and 8,440,432, each of which are incorporated by reference in their entirety.

By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In some embodiments, the nucleic acid is DNA.

As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers.

As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the disclosure, polypeptide monomers with an RVD of IG preferentially bind to T. Thus, the number and order of the polypeptide monomer repeats in the nucleic acid binding domain of a TALE determines its nucleic acid target specificity. In still further embodiments of the disclosure, polypeptide monomers with an RVD of NS recognize all four base pairs and may bind to A, T, G or C. The structure and function of TALEs is further described in, for example, Moscou et al., Science 326: 1501 (2009); Boch et al., Science 326: 1509-1512 (2009); and Zhang et al., Nature Biotechnology 29: 149-153 (2011), each of which is incorporated by reference in its entirety. In certain embodiments, targeting is effected by a polynucleic acid binding TALEN fragment. In certain embodiments, the targeting domain comprises or consists of a catalytically inactive TALEN or nucleic acid binding fragment thereof.

### Zn-Finger Nuclease (ZFN) System

In certain embodiments, the nucleic acid binding protein (e.g., DNA binding protein) comprises or consists of a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, each of which are incorporated by reference in their entirety. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. In certain embodiments, the targeting domain comprises or consists of a nucleic acid binding zinc finger nuclease or a nucleic acid binding fragment thereof. In certain embodiments, the nucleic acid binding (fragment of) a zinc finger nuclease is catalytically inactive.

### Meganuclease

In certain embodiments, the nucleic acid binding protein (e.g., DNA binding protein) comprises a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, each of which are incorporated by reference in their entirety. In certain embodiments, targeting is effected by a polynucleic acid binding meganuclease fragment. In certain embodiments, targeting is effected by a polynucleic acid binding catalytically inactive meganuclease (fragment). Accordingly in particular embodiments, the targeting domain comprises or consists of a nucleic acid binding meganuclease or a nucleic acid binding fragment thereof.

### CRISPR-Cas Systems

In some embodiments, the nucleic acid binding protein (e.g., DNA binding protein) and single guide RNA sequence of the present disclosure are derived from the CRISPR-Cas system. The present disclosure provides CRISPR/Cas9-based engineered systems for use in genome editing and treating genetic diseases. The CRISPR/Cas9-based engineered systems may be designed to target any gene (e.g. PCSK9), including genes involved in a genetic disease, liver disease and dysregulation of cholesterol such as LDL. The present disclosure provides a CRISPR-Cas system comprising genetically engineered Cas proteins and/or guide RNAs with desired specificity and activity (e.g. reducing or eliminating expression of a PCSK9 gene product). The CRISPR/Cas9-based systems may include a Cas9 protein, a mutated Cas9 protein or Cas9 fusion protein (e.g. DNMT3A-DNMT3L(3A3L)-dCas9-KRAB fusion molecule) and at least one sgRNA (e.g. PCSK9 sgRNA). The Cas9 fusion protein may, for example, include a domain that has a different activity from what is endogenous to Cas9 (e.g. DNMT3A, DNMT3L or KRAB).

In general, a Cas protein (used interchangeably herein with CRISPR protein, CRISPR enzyme, CRISPR-Cas protein, CRISPR-Cas enzyme, Cas, CRISPR effector, or Cas effector protein) and/or a guide sequence is a component of a CRISPR-Cas system. A CRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, e.g. CRISPR RNA and transactivating (tracr) RNA or a single guide RNA (aka sgRNA; chimeric RNA) or other sequences and transcripts from a CRISPR locus.

In general, a CRISPR system is characterized by elements that promote the formation of a CRISPR complex at the site of a target sequence (also referred to as a protospacer in the context of an endogenous CRISPR system). In an engineered system of the disclosure, the direct repeat may encompass naturally occurring sequences or non-naturally occurring sequences. The direct repeat of the disclosure is not limited to naturally occurring lengths and sequences. Furthermore, a direct repeat of the disclosure may include insertions of nucleotides such as an aptamer or sequences that bind to an adapter protein (for association with functional domains). In certain embodiments, one end of a direct repeat containing such as an insertion is roughly the first half of a short DR and the end is roughly the second half of the short DR.

In the context of formation of a CRISPR complex, “target sequence” or “target polynucleotides” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

In general, a guide sequence (or spacer sequence) may be any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 70%, 75%, 80%, 85%, 90%, 91%, 92%, 93%, 94%, 95%, 96%, 97%, 98%, 99%, or more.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance, a double mismatch is, the more cleavage efficiency is affected. Accordingly, by choosing mismatch positions along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

A CRISPR-Cas system or components thereof may be used for introducing one or more mutations in a target locus or nucleic acid sequence. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s).

Typically, in the context of an endogenous CRISPR-Cas system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In some cases, in the context of an endogenous CRISPR system, formation of a CRISPR complex (comprising a guide sequence hybridized to a target sequence and complexed with one or more Cas proteins) results in cleavage of one or both strands (if applicable) in or near (e.g. within 1, 2, 3, 4, 5, 6, 7, 8, 9, 10, 20, 50, or more base pairs from) the target sequence.

In some embodiments, the guide RNA (capable of guiding Cas to a target locus) may comprise (1) a guide sequence capable of hybridizing to a target locus (a polynucleotide target locus, such as an RNA target locus) in the eukaryotic cell; (2) a direct repeat (DR) sequence, which reside in a single RNA, i.e. an sgRNA (arranged in a 5′ to 3′ orientation) or crRNA.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant disclosure, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publication Nos. US 2014-0310830, US 2014-0287938 A1, US 2014-0273234 A1, US 2014-0273232 A1, US 2014-0273231 A1, US 2014-0256046 A1, US 2014-0248702 A1, US 2014-0242700 A1, US 2014-0242699 A1, US 2014-0242664 A1, US 2014-0234972 A1, US 2014-0227787 A1, US 2014-0189896 A1, US 2014-0186958, US 2014-0186919 A1, US 2014-0186843 A1, US 2014-0179770 A1 and US 2014-0179006 A1, US 2014-0170753; European Patents EP 2784162 B1 and EP 2771468 B1; European Patent Applications EP 2771468, EP 2764103, and EP 2784162; and PCT Patent Publications WO 2021/183807A1 (PCT/US2021/021973), WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), each of which are incorporated herein by reference in their entirety.

### Cas Proteins

The Cas protein (e.g., engineered Cas protein) may have a nuclease activity that is substantially the same (e.g., between 80% and 100%, between 90% and 100%, between 95% and 100%, between 98% and 100%, between 99% and 100%, between 99.9% and 100%, or about 100%) as a wildtype counterpart Cas protein. In certain cases, the engineered Cas protein has a nuclease activity that is higher than (e.g., at least 5%, at least 10%, at least 20%, at least 30%, at least 40%, at least 50%, at least 60%, at least 70%, at least 80%, or at least 90% higher than) a wildtype counterpart Cas protein.

Alternatively or additionally, the Cas protein (e.g., engineered Cas protein) may have a specificity at least 5%, at least 10%, at least 20%, at least 30%, at least 40%, at least 50%, at least 60%, at least 70%, at least 80%, or at least 90% higher than the wildtype counterpart Cas protein. In a particular example, the Cas protein (e.g., engineered Cas protein) may have a specificity at least 30% higher than the wildtype counterpart Cas protein. As used herein, the term “specificity” of a Cas may correspond to the number or percentage of on-target polynucleotide cleavage events relative to the number or percentage of all polynucleotide cleavage events, including on-target and off-target events. The activity and specificity of a Cas protein are consistent with those described in Hsu P D et al., DNA targeting specificity of RNA-guided Cas9 nucleases, Nat Biotechnol. 2013 September; 31(9): 827-832; and Slaymaker I M, et al., Rationally engineered Cas9 nucleases with improved specificity, Science. 2016 Jan. 1; 351(6268): 84-88, which also describe examples of methods for detecting the activity and specificity of Cas proteins, and are incorporated herein by reference in their entireties, and are detailed elsewhere herein.

In some embodiments, the Cas protein (e.g., its RuvC domain) may slide one base upstream (with respect to the PAM), and produce a staggered cut, which may be filled and lead to duplication of a single base (i.e., +1 insertion). An example of a +1 insertion position is described in Zuo, Z., and Liu, J. (2016). Cas9-catalyzed DNA Cleavage Generates Staggered Ends: Evidence from Molecular Dynamics Simulations. Scientific Reports 6, 37584. In some embodiments, the engineered Cas protein has a +1 insertion frequency different from the wildtype counterpart Cas protein. For example, the +1 insertion frequency when a guanine is present in the −2 position with respect a PAM is higher than the +1 insertion frequency when a thymidine, a cytidine, or a adenine is present in the −2 position with respect the PAM. In some cases, the +1 insertions depend on host machinery in human cells. In some examples, the Cas protein may generate a staggered cut. The staggered cut may be a 1-bp or 1-nucleotide 5′ overhang. The staggered cut may be a 1-bp or 1-nucleotide 3′ overhang.

The nucleic acid molecule encoding a Cas may be codon optimized. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in tum believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In some embodiments, the Cas proteins may have nucleic acid cleavage activity. The Cas proteins may have RNA binding and DNA cleaving function. In some embodiments, Cas may direct cleavage of one or two nucleic acid strands at the location of or near a target sequence, such as within the target sequence and/or within the complement of the target sequence or at sequences associated with the target sequence, e.g., within about 1, 2, 3, 4, 5, 6, 7, 8, 9, 10, 15, 20, 25, 50, 100, 200, 500, or more base pairs from the first or last nucleotide of a target sequence. In some embodiments, the Cas protein may direct more than one cleavage (such as one, two three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt, i.e., generating blunt ends. In some embodiments, the cleavage may be staggered, i.e., generating sticky ends.

In some embodiments, a vector encodes a nucleic acid-targeting Cas protein that may be mutated with respect to a corresponding wild-type enzyme such that the mutated nucleic acid-targeting Cas protein lacks the ability to cleave one or two strands of a target polynucleotide containing a target sequence, e.g., alteration or mutation in a HNH domain to produce a mutated Cas substantially lacking all DNA cleavage activity, e.g., the DNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “derived” with reference to an enzyme means that the derived enzyme is largely based, in the sense of having a high degree of sequence homology with, a wildtype enzyme, but that it has been mutated (modified) in some way as known in the art or as described herein.

Typically, in the context of an endogenous nucleic acid-targeting system, formation of a nucleic acid-targeting complex (comprising a guide RNA or crRNA hybridized to a target sequence and complexed with one or more nucleic acid-targeting effector proteins) results in cleavage of DNA strand(s) in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

It will be appreciated that the effector protein is based on or derived from an enzyme, so the term “effector protein” certainly includes “enzyme” in some embodiments. However, it will also be appreciated that the effector protein may, as required in some embodiments, have DNA or RNA binding, but not necessarily cutting or nicking, activity, including a dead-Cas protein function.

In some embodiments, a Cas protein may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the CRISPR effector protein may be a part of a Light Inducible Transcriptional Effector (LITE) to direct changes in transcriptional activity in a sequence-specific manner. The components of a light may include a CRISPR effector protein, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, and WO 2014018423 A2 which are hereby incorporated by reference in their entirety.

In some embodiments, a mutated Cas may have one or more mutations resulting in reduced off-target effects, e.g., improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the disclosure as described herein elsewhere. Any of the methods, products, compositions and uses as described herein elsewhere are equally applicable with the mutated CRISPR enzymes as further detailed below.

The methods and mutations which can be employed in various combinations to increase or decrease activity and/or specificity of on-target vs. off-target activity, or increase or decrease binding and/or specificity of on-target vs. off-target binding, can be used to compensate or enhance mutations or modifications made to promote other effects. Such mutations or modifications made to promote other effects in include mutations or modification to the Cas and or mutation or modification made to a guide RNA. The methods and mutations of the disclosure are used to modulate Cas nuclease activity and/or binding with chemically modified guide RNAs.

In certain embodiments, the catalytic activity of the Cas protein of the disclosure is altered or modified. It is to be understood that mutated Cas has an altered or modified catalytic activity if the catalytic activity is different than the catalytic activity of the corresponding wild type Cas protein (e.g., unmutated Cas protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, at least 10%, at least 20%, at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially decrease all catalytic activity, decrease activity to below detectable levels, or decrease to no measurable catalytic activity.

One or more characteristics of the engineered Cas protein may be different from a corresponding wildtype Cas protein. Examples of such characteristics include catalytic activity, gRNA binding, specificity of the Cas protein (e.g., specificity of editing a defined target), stability of the Cas protein, off-target binding, target binding, protease activity, nickase activity, PFS recognition. In some examples, a engineered Cas protein may comprise one or more mutations of the corresponding wild type Cas protein. In some embodiments, the catalytic activity of the engineered Cas protein is increased as compared to a corresponding wildtype Cas protein. In some embodiments, the catalytic activity of the engineered Cas protein is decreased as compared to a corresponding wildtype Cas protein. In some embodiments, the gRNA binding of the engineered Cas protein is increased as compared to a corresponding wildtype Cas protein. In some embodiments, the gRNA binding of the engineered Cas protein is decreased as compared to a corresponding wildtype Cas protein. In some embodiments, the specificity of the Cas protein is increased as compared to a corresponding wildtype Cas protein. In some embodiments, the specificity of the Cas protein is decreased as compared to a corresponding wildtype Cas protein. In some embodiments, the stability of the Cas protein is increased as compared to a corresponding wildtype Cas protein. In some embodiments, the stability of the Cas protein is decreased as compared to a corresponding wildtype Cas protein. In some embodiments, the engineered Cas protein further comprises one or more mutations which inactivate catalytic activity. In some embodiments, the off-target binding of the Cas protein is increased as compared to a corresponding wildtype Cas protein. In some embodiments, the off-target binding of the Cas protein is decreased as compared to a corresponding wildtype Cas protein. In some embodiments, the target binding of the Cas protein is increased as compared to a corresponding wildtype Cas protein. In some embodiments, the target binding of the Cas protein is decreased as compared to a corresponding wildtype Cas protein. In some embodiments, the engineered Cas protein has a higher protease activity or polynucleotide-binding capability compared with a corresponding wildtype Cas protein. In some embodiments, the PFS recognition is altered as compared to a corresponding wildtype Cas protein.

### Examples of Cas Proteins

Examples of Cas proteins include those of Class I (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d,), CasX, CasY, Cas14, variants thereof (e.g., mutated forms, truncated forms), homologs thereof, and orthologs thereof. The terms “ortholog” and “homolog” are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Class 2 Cas Proteins

In some embodiments, the Cas protein is a class 2 Cas protein, i.e., a Cas protein of a class 2 CRISPR-Cas system. A class 2 CRISPR-Cas system may be of a subtype, e.g., Type II-A, Type II-B, Type II-C, Type V-A, Type V-B, Type V-C, or Type V-U. In some embodiments, the Cas protein is Cas9, Cas12a, Cas12b, Cas12c, or Cas12d. In some embodiments, Cas9 may be SpCas9, SaCas9, StCas9 and other Cas9 orthologs. Cas12 may be Cas12a, Cas12b, and Cas12c, including FnCas12a, or homology or orthologs thereof. The definition and exemplary members of the CRISPR-Cas system include those described in Kira S. Makarova and Eugene V. Koonin, Annotation and Classification of CRISPR-Cas systems, Methods Mol Biol. 2015; 1311: 47-75; and Sergey Shmakov et al., Diversity and evolution of class 2 CRISPR-Cas systems, Nat Rev Microbial. 2017 March; 15(3): 169-182.

### Cas Protein Linkers

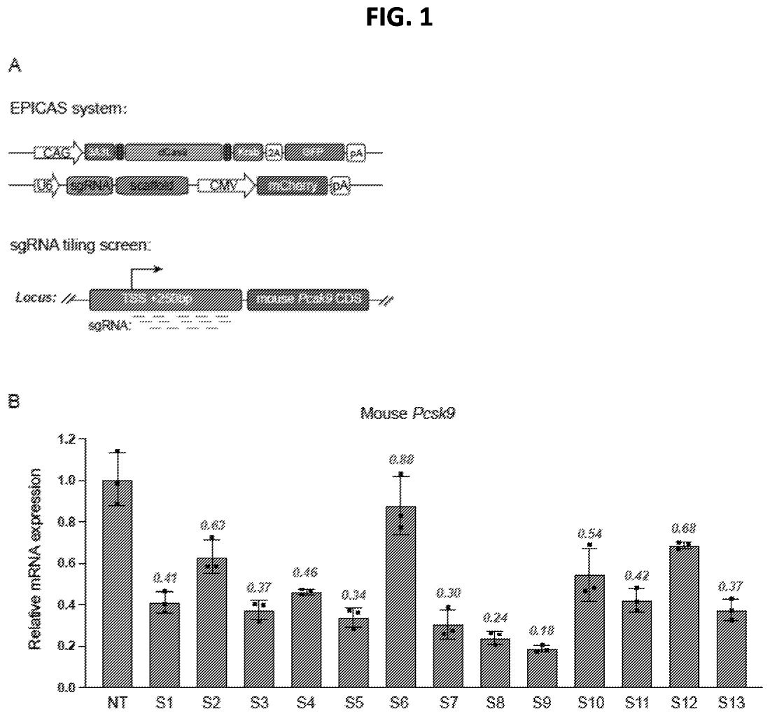

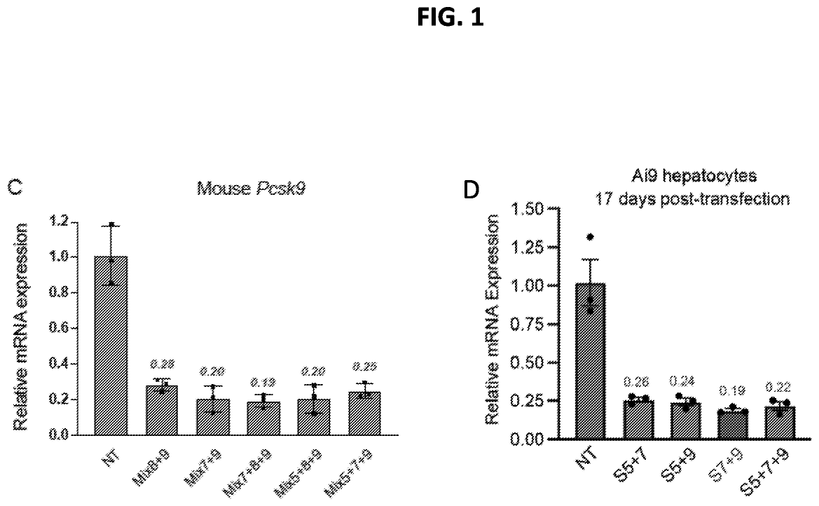

In some examples, the Cas protein comprises at least one RuvC domain and at least one HNH domain. The Cas protein may further comprise a first and a second linker domain connecting the RuvC domain and the HNH domain. The first linker (L1) and second linker (L2) connecting the HNH and RuvC domains in Cas9 are described in studies by Nishimasu, H. et al. “Crystal structure of Cas9 in complex with guide RNA and target RNA” Cell 156 (Feb. 27, 2014): 935-949 and Ribeiro, L. et al. (2018) “Protein engineering strategies to expand CRISPR-Cas9 applications” International Journal of Genomics Volume 2018, Article ID 1652567 (doi.org/10.1155/2018/1652567). FIG. 1 of Ribeiro shows the overall organization, structure and function of Cas9, incorporated specifically herein by reference. Specifically, FIG. 1A shows a schematic representation of the domain organization of SpCas9 indicating the genetic architecture of the HNH and RuvC domains including the linkers L1 (spanning amino acids 765-780) and L2 (spanning amino acids 906-918) as described herein.

Similarly, the domain organization of Staphylococcus aureus Cas9 (SaCas9) can be utilized when referencing the first and second linker domains. In an aspect, the Linker 1 domain region spans residues 481-519, and connects the RuvC-II domain to the HNH domain in SaCas9. In some embodiments, Linker 2 region spans residues 629-649, and connects the RuvC-III domain and the HNH domain of SaCas9. Accordingly, the first and/or second linker domain may be mutated in a Cas9 ortholog, and reference may be made to amino acid residues corresponding to the amino acids of a wild-type SaCas9. See, Nishimasu, Cell. 2015 Aug. 27; 162(5): 1113-1126; doi: 10.1016/j.cell.2015.08.007, incorporated by reference herein. In particular, FIG. 1, S1-S3 of Nishimasu detail domain organization of Cas9 proteins, and are incorporated specifically by reference herein for their teachings.

The first and second linker may comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acids. The first and second linker may correspond to wild-type linkers. In some aspects, the first and second linkers may comprise one or more mutations in the first and/or second linker. In an aspect the first and/or second linker comprise one or more mutations that improve specificity of the Cas9 protein.

In some embodiments, the linkers, L1 and L2, connecting the HNH and RuvC domains of Cas9 contain the wild-type amino acid sequences. In some embodiments, the linkers connecting the HNH and RuvC domains contain mutations in one or more amino acids. In an example embodiment, the first linker (L1) contains the mutation corresponding to amino acid T769I of SpCas9 and/or the second linker (L2) contains the mutation corresponding to amino acid G915M of SpCas9. In an example embodiment, one or more linker mutations, e.g., T769I and G915M, confer improved specificity upon the Cas9 protein.

In one embodiment, one or mutations in the first and second linker may be combined with one or more mutations in other portions of the Cas9 protein for further improved specificity and/or retention of activity that is substantially equivalent to a wild-type Cas9 protein, as described herein. In one embodiment, mutations in the linker and/or additional mutations within the Cas protein can be identified utilizing the methods detailed herein that enhance/improve specificity and substantially retain wild-type activity to the wild-type Cas9.

Class 2, Type H Cas Proteins (e.g. Cas9)

In some embodiments, the Cas protein may be a Cas protein of a Class 2, Type II CRISPR-Cas system (a Type II Cas protein). In some embodiments, the Cas protein may be a class 2 Type II Cas protein, e.g., Cas9. In some embodiments, the CRISPR/Cas9-based system may include a Cas9 protein or a fragment thereof, a Cas9 fusion protein, a nucleic acid encoding a Cas9 protein or a fragment thereof, or a nucleic acid encoding a Cas9 fusion protein. By “Cas9 (CRISPR associated protein 9)” is meant a polypeptide or fragment thereof having at least about 85% amino acid identity to NCBI Accession No. NP_269215 and having RNA binding activity, DNA binding activity, and/or DNA cleavage activity (e.g., endonuclease or nickase activity). “Cas9 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cas 9 nucleic acid molecule” is meant a polynucleotide encoding a Cas9 polypeptide or fragment thereof. An exemplary Cas9 nucleic acid molecule sequence is provided at genome sequence No. NC_002737. In some embodiments, disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

In some cases, the CRISPR-Cas protein is Cas9 or a variant thereof. In some examples, Cas9 may be wildtype Cas9 including any naturally occurring bacterial Cas9. Cas9 orthologs typically share the general organization of 3-4 RuvC domains and a HNH domain. The 5′ most RuvC domain cleaves the non-complementary strand, and the HNH domain cleaves the complementary strand. All notations are in reference to the guide sequence. The catalytic residue in the 5′ RuvC domain is identified through homology comparison of the Cas9 of interest with other Cas9 orthologs (from S. pyogenes type II CRISPR locus, S. thermophilus CRISPR locus 1, S. thermophilus CRISPR locus 3, and Franciscilla novicida type II CRISPR locus), and the conserved Asp residue (D10) is mutated to alanine to convert Cas9 into a complementary-strand nicking enzyme. Accordingly, the Cas enzyme can be wildtype Cas9 including any naturally occurring bacterial Cas9. The CRISPR, Cas or Cas9 enzyme can be codon optimized, or a modified version, including any chimaeras, mutants, homologs or orthologs. In an additional aspect of the disclosure, a Cas9 enzyme may comprise one or more mutations and may be used as a generic DNA binding protein with or without fusion to a functional domain.

The mutations may be artificially introduced mutations or gain-of-function or loss-of-function mutations. In some embodiments, the transcriptional activation domain may be VP64. In some embodiments, the transcriptional repressor domain may be KRAB or SID4X. Other aspects of the disclosure relate to the mutated Cas9 enzyme being fused to domains which include but are not limited to a nuclease, a transcriptional activator, repressor, a recombinase, a transposase, a histone remodeler, a demethylase, a DNA methyltransferase, a cryptochrome, a light inducible/controllable domain or a chemically inducible/controllable domain. The disclosure can involve sgRNAs or tracrRNAs or guide or chimeric guide sequences that allow for enhancing performance of these RNAs in cells. This type II CRISPR enzyme may be any Cas enzyme. In some cases, the Cas9 enzyme is from, or is derived from, SpCas9 or SaCas9. As used herein, the term “derived” with reference to an enzyme means that the derived enzyme is largely based, in the sense of having a high degree of sequence homology with, a wildtype enzyme, but that it has been mutated (modified) in some way as known in the art or as described herein. In an example the mutation may comprise one or more mutations in a first linker domain, a second linker domain, and/or other portions of the protein. The high degree of sequence homology may comprise at least 80%, 85%, 86%, 87%, 88%, 89%, 90%, 91%, 92%, 93%, 94%, 95%, 96%, 97%, 98%, 99% or more relative to a wildtype enzyme.

A Cas enzyme may be identified Cas9 as this can refer to the general class of enzymes that share homology to the biggest nuclease with multiple nuclease domains from the type II CRISPR system. In some cases, the Cas9 enzyme is from, or is derived from, SpCas9 (S. pyogenes Cas9) or saCas9 (S. aureus Cas9). “StCas9” refers to wildtype Cas9 from S. thermophilus (UniProt ID: G3ECR1). Similarly, “SpCas9” refers to wildtype Cas9 from S. pyogenes (UniProt ID: Q99ZW2). As used herein, the term “derived” with reference to an enzyme means that the derived enzyme is largely based, in the sense of having a high degree of sequence homology with, a wildtype enzyme, but that it has been mutated (modified) in some way as known in the art or as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes.

In particular embodiments, the DNA binding protein is a Cas9 protein from or originated from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacte, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter.

In some embodiments, the Cas9 protein is from or originated from an organism selected from S. mutans, S. agalactiae, S. equisimilis, S. sanguinis, S. pneumonia, C. jejuni, C. coli; N salsuginis, N tergarcus; S. auricularis, S. carnosus; N meningitides, N gonorrhoeae, L. monocytogenes, L. ivanovii; C. botulinum, C. difficile, C. tetani, or C. sordellii, Francisella tularensis 1, Francisella tularensis subsp. novicida, Prevotella albensis, Lachnospiraceae bacterium MC20171, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. In some embodiments, Cas9 protein is from an organism from or originated from Streptococcus pyogenes, Staphylococcus aureus, or Streptococcus thermophilus Cas9.

In a more preferred embodiment, the Cas9 protein is derived from a bacterial species selected from Streptococcus pyogenes, Staphylococcus aureus, or Streptococcus thermophilus Cas9. In certain embodiments, the Cas9 is derived from a bacterial species selected from Francisella tularensis 1, Prevotella albensis, Lachnospiraceae bacterium MC20171, Butyrivibrio proteoclasticus, Peregrinibacteria bacterium GW2011 GWA2 33 JO, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cas9 protein is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

Cas9 enzymes include but are not limited to S. pyogenes serotype M1 (UniProt ID: Q99ZW2), S. aureus Cas9 (UniProt ID: J7RUA5), Eubacterium ventriosum Cas9 (UniProt ID: A5Z395), Azospirillum (strain B510) Cas9 (UniProt ID: D3NT09), Gluconacetobacter diazotrophicus (strain ATCC 49037) Cas9 (UnitProt ID: A9HKP2), Nisseria cinerea Cas9 (UniProt ID: DOW2Z9), Roseburia intestinalis Cas9 (UniProt ID: C7G697), Parvibaculum lavamentivorans (strain DS-1) Cas9 (UniProt ID: A7HP89), Nitratifractor salsuginis (strain DSM 16511) Cas9 (UniProt ID: E6WZS9), Campylobacter lari Cas9 (UniProt ID: G1UFN3).

Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archaea, Mole Cell 2010, January 15; 37(1): 7. The type II CRISPR locus from Streptococcus pyogenes SF370, which contains a cluster of four genes Cas9, Cas1, Cas2, and Csnl, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions. One can generate chimeric Cas9 proteins and Cas9 may be used as a generic DNA binding protein. The structural information provided for Cas9 may be used to further engineer and optimize the CRISPR-Cas system and this may be extrapolated to interrogate structure-function relationships in other CRISPR enzyme systems as well, particularly structure-function relationships in other Type II CRISPR enzymes or Cas9 orthologs. The crystal structure information (described in U.S. provisional applications 61/915,251 filed Dec. 12, 2013, 61/930,214 filed on Jan. 22, 2014, 61/980,012 filed Apr. 15, 2014; and Nishimasu et al, “Crystal Structure of Cas9 in Complex with Guide RNA and Target DNA,” Cell 156(5):935-949, DOI: http://dx.doi.org/10.1016/j.cell.2014.02.001 (2014), each and all of which are incorporated herein by reference) provides structural information to truncate and create modular or multi-part CRISPR enzymes which may be incorporated into inducible CRISPR-Cas systems. In particular, structural information is provided for S. pyogenes Cas9 (SpCas9) and this may be extrapolated to other Cas9 orthologs or other Type II CRISPR enzymes. The Cas9 gene is found in several diverse bacterial genomes, typically in the same locus with cas1, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region.

dCas9

The Cas9 protein may be mutated so that the nuclease activity is inactivated. An inactivated Cas9 protein from S. pyogenes (iCas9, also referred to as “dCas9”) with no endonuclease activity has been recently targeted to genes in bacteria, yeast, and human cells by gRNA to silence gene expression through steric hindrance. As used herein, a “dCas molecule” may refer to a dCas protein, or a fragment thereof. As used herein, a “dCas9 molecule” may refer to a dCas9 protein, or a fragment thereof. As used herein, the terms “iCas” and “dCas” are used interchangeably and refer to a catalytically inactive CRISPR associated protein. In one embodiment, the dCas molecule comprises one or more mutations in a DNA-cleavage domain. In one embodiment, the dCas molecule comprises one or more mutations in the RuvC or HNH domain. In one embodiment, the dCas molecule comprises one or more mutations in both the RuvC and HNH domain. In one embodiment, the dCas molecule is a fragment of a wild-type Cas molecule. In one embodiment, the dCas molecule comprises a functional domain from a wild-type Cas molecule, wherein the functional domain is chosen from a Reel domain, a bridge helix domain, or a PAM interacting domain. In one embodiment, the nuclease activity of the dCas molecule is reduced by at least 40%, 45%, 50%, 55%, 60%, 65%, 70%, 75%, 80%, 85%, 90%, 95%, or 99% compared to that of a corresponding wild type Cas molecule.

Suitable dCas molecule can be derived from a wild type Cas molecule. The Cas molecule can be from a type I, type II, or type III CRISPR-Cas systems. In one embodiment, suitable dCas molecules can be derived from a Cas1, Cas2, Cas3, Cas4, Cas5, Cas6, Cas7, Cas8, Cas9, or Cas10 molecule. In one embodiment, the dCas molecule is derived from a Cas9 molecule. The dCas9 molecule can be obtained, for example, by introducing point mutations (e.g., substitutions, deletions, or additions) in the Cas9 molecule at the DNA-cleavage domain, e.g., the nuclease domain, e.g., the RuvC and/or HNH domain. See, e.g., Jinek et al., Science (2012) 337:816-21, incorporated by reference herein in its entirety. For example, introducing two point mutations in the RuvC and HNH domains reduces the Cas9 nuclease activity while retaining the Cas9 sgRNA and DNA binding activity. In one embodiment, the two point mutations within the RuvC and HNH active sites are D10A and H840A mutations of the S. pyogenes Cas9 molecule. Alternatively, D10 and H840 of the S. pyogenes Cas9 molecule can be deleted to abolish the Cas9 nuclease activity while retaining its sgRNA and DNA binding activity. In one embodiment, the two point mutations within the RuvC and HNH active sites are D10A and N580A mutations of the S. pyogenes Cas9 molecule.

In one embodiment, the dCas molecule is an S. aureus dCas9 molecule comprising a mutation at D10 and/or N580, numbered according to SEQ ID NO: 1. In one embodiment, the dCas molecule is an S. aureus dCas9 molecule comprising D10A and/or N580A mutations, numbered according to SEQ ID NO: 1.

S. aureus dCas9
(SEQ ID NO: 1)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLINLGAPAAFKYFDTTIDRKRYTSTKEVLDATLIHQSITGLYETRIDLSQ
LGGD

In one embodiment, the dCas9 molecule is an S. aureus dCas9 molecule comprising the amino acid sequence of SEQ ID NO: 2 or 3, a sequence substantially identical (e.g., at least 80%, 85%, 90%, 91%, 92%, 93%, 94%, 95%, 96%, 97%, 98%, 99% or higher in sequence identity) to SEQ ID NO: 2 or 3, or a sequence having one, two, three, four, five or more changes, e.g., amino acid substitutions, insertions, or deletions, relative to SEQ ID NO: 2 or 3, or any fragment thereof.

Similar mutations can also apply to any other naturally-occurring Cas9 (e.g., Cas9 from other species) or engineered Cas9 molecules. In certain embodiments, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B 510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 1651 1) dCas9 molecule, a Campylobacter lari (strain CF89-12) dCas9 molecule, a Streptococcus thermophilus (strain LMD-9) dCas9 molecule, or fragment thereof.

In certain embodiments, the present disclosure provides a vector comprising a nucleotide encoding a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 1651 1) dCas9 molecule, a Campylobacter lari (strain CF89-12) dCas9 molecule, a Streptococcus thermophilus (strain LMD-9) dCas9 molecule, or fragment thereof.

Exemplary dCas9 proteins include but are not limited to those listed in Table 1.

TABLE 1
Exemplary dCas9 proteins

| Description | Sequence | SEQ ID NO: |
|---|---|---|
| Staphylococcus | MKRNYILGLAIGITSVGYGIIDYETRDVIDAGVRLFKEANVENNEGR | SEQ ID NO: |
| aureus | RSKRGARRLKRRRRHRIQRVKKLLFDYNLLTDHSELSGINPYEARVK | 2 |
| dCas9 | GLSQKLSEEEFSAALLHLAKRRGVHNVNEVEEDTGNELSTKEQISRN | |
| (Mutant: | SKALEEKYVAELQLERLKKDGEVRGSINRFKTSDYVKEAKQLLKVQK | |
| D10A and | AYHQLDQSFIDTYIDLLETRRTYYEGPGEGSPFGWKDIKEWYEMLMG | |
| H557A) | HCTYFPEELRSVKYAYNADLYNALNDLNNLVITRDENEKLEYYEKFQ | |
| | IIENVFKQKKKPTLKQIAKEILVNEEDIKGYRVISTGKPEFTNLKVY | |
| | HDIKDITARKEIIENAELLDQIAKILTIYQSSEDIQEELTNLNSELT | |
| | QEEIEQISNLKGYTGTHNLSLKAINLILDELWHINDNQIAIFNRLKL | |
| | VPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENA | |
| | KYLIEKIKLHDMQEGKCLYSLEAIPLEDLLNNPFNYEVDAIIPRSVS | |
| | FDNSFNNKVLVKQEENSKKGNRTPFQYLSSSDSKISYETFKKHILNL | |
| | AKGKGRISKTKKEYLLEERDINRFSVQKDFINRNLVDTRYATRGLMN | |
| | LLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKE | |
| | IFITPHQIKHIKDFKDYKYSHRVDKKPNRELINDTLYSTRKDDKGNT | |
| | LIVNNLNGLYDKDNDKLKKLINKSPEKLLMYHHDPQTYQKLKLIMEQ | |
| | YGDEKNPLYKYYEETGNYLTKYSKKDNGPVIKKIKYYGNKLNAHLDI | |
| | TDDYPNSRNKVVKLSLKPYRFDVYLDNGVYKFVTVKNLDVIKKENYY | |
| | EVNSKCYEEAKKLKKISNQAEFIASFYNNDLIKINGELYRVIGVNND | |
| | LLNRIEVNMIDITYREYLENMNDKRPPRIIKTIASKTQSIKKYSTDI | |
| | LGNLYEVKSKKHPQIIKKG | |
| Staphylococcus | MKRNYILGLAIGITSVGYGIIDYETRDVIDAGVRLFKEANVENNEGR | SEQ ID NO: |
| aureus | RSKRGARRLKRRRRHRIQRVKKLLFDYNLLTDHSELSGINPYEARVK | 3 |
| dCas9 | GLSQKLSEEEFSAALLHLAKRRGVHNVNEVEEDTGNELSTKEQISRN | |
| (Mutant: | SKALEEKYVAELQLERLKKDGEVRGSINRFKTSDYVKEAKQLLKVQK | |
| D10A and | AYHQLDQSFIDTYIDLLETRRTYYEGPGEGSPFGWKDIKEWYEMLMG | |
| N580A) | HCTYFPEELRSVKYAYNADLYNALNDLNNLVITRDENEKLEYYEKFQ | |
| | IIENVFKQKKKPTLKQIAKEILVNEEDIKGYRVTSTGKPEFTNLKVY | |
| | HDIKDITARKEIIENAELLDQIAKILTIYQSSEDIQEELTNLNSELT | |
| | QEEIEQISNLKGYTGTHNLSLKAINLILDELWHINDNQIAIFNRLKL | |
| | VPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENA | |
| | KYLIEKIKLHDMQEGKCLYSLEAIPLEDLLNNPFNYEVHAIIPRSVS | |
| | FDNSFNNKVLVKQEEASKKGNRTPFQYLSSSDSKISYETFKKHILNL | |
| | AKGKGRISKTKKEYLLEERDINRFSVQKDFINRNLVDTRYATRGLMN | |
| | LLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKE | |
| | IFITPHQIKHIKDFKDYKYSHRVDKKPNRELINDTLYSTRKDDKGNT | |
| | LIVNNLNGLYDKDNDKLKKLINKSPEKLLMYHHDPQTYQKLKLIMEQ | |
| | YGDEKNPLYKYYEETGNYLTKYSKKDNGPVIKKIKYYGNKLNAHLDI | |
| | TDDYPNSRNKVVKLSLKPYRFDVYLDNGVYKFVTVKNLDVIKKENYY | |
| | EVNSKCYEEAKKLKKISNQAEFIASFYNNDLIKINGELYRVIGVNND | |
| | LLNRIEVNMIDITYREYLENMNDKRPPRIIKTIASKTQSIKKYSTDI | |
| | LGNLYEVKSKKHPQIIKKG | |
| Streptococcus | MKRNYILGLAIGITSVGYGIIDYETRDVIDAGVRLFKEANVENNEGR | SEQ ID NO: |
| pyogenes | RSKRGARRLKRRRRHRIQRVKKLLFDYNLLTDHSELSGINPYEARVK | 4 |
| dCas9 | GLSQKLSEEEFSAALLHLAKRRGVHNVNEVEEDTGNELSTKEQISRN | |
| (D10A, | SKALEEKYVAELQLERLKKDGEVRGSINRFKTSDYVKEAKQLLKVQK | |
| H840A) | AYHQLDQSFIDTYIDLLETRRTYYEGPGEGSPFGWKDIKEWYEMLMG | |
| | HCTYFPEELRSVKYAYNADLYNALNDLNNLVITRDENEKLEYYEKFQ | |
| | IIENVFKQKKKPTLKQIAKEILVNEEDIKGYRVTSTGKPEFTNLKVY | |
| | HDIKDITARKEIIENAELLDQIAKILTIYQSSEDIQEELTNLNSELT | |
| | QEEIEQISNLKGYTGTHNLSLKAINLILDELWHINDNQIAIFNRLKL | |
| | VPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENA | |
| | KYLIEKIKLHDMQEGKCLYSLEAIPLEDLLNNPFNYEVHAIIPRSVS | |
| | FDNSFNNKVLVKQEEASKKGNRTPFQYLSSSDSKISYETFKKHILNL | |
| | AKGKGRISKTKKEYLLEERDINRFSVQKDFINRNLVDTRYATRGLMN | |
| | LLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKE | |
| | IFITPHQIKHIKDFKDYKYSHRVDKKPNRELINDTLYSTRKDDKGNT | |
| | LIVNNLNGLYDKDNDKLKKLINKSPEKLLMYHHDPQTYQKLKLIMEQ | |
| | YGDEKNPLYKYYEETGNYLTKYSKKDNGPVIKKIKYYGNKLNAHLDI | |
| | TDDYPNSRNKVVKLSLKPYRFDVYLDNGVYKFVTVKNLDVIKKENYY | |
| | EVNSKCYEEAKKLKKISNQAEFIASFYNNDLIKINGELYRVIGVNND | |
| | LLNRIEVNMIDITYREYLENMNDKRPPRIIKTIASKTQSIKKYSTDI | |
| | LGNLYEVKSKKHPQIIKKG | |
| Campylobacter | MARILAFAIGISSIGWAFSENDELKDCGVRIFTKVENPKTGESLALP | SEQ ID NO: |
| jejuni | RRLARSARKRLARRKARLNHLKHLIANEFKLNYEDYQSFDESLAKAY | 5 |
| dCas9 | KGSLISPYELRFRALNELLSKQDFARVILHIAKRRGYDDIKNSDDKE | |
| (D8A, | KGAILKAIKQNEEKLANYQSVGEYLYKEYFQKFKENSKEFTNVRNKK | |
| H559A) | ESYERCIAQSFLKDELKLIFKKQREFGFSFSKKFEEEVLSVAFYKRA | |
| | LKDFSHLVGNCSFFTDEKRAPKNSPLAFMFVALTRIINLLNNLKNTE | |
| | GILYTKDDLNALLNEVLKNGTLTYKQTKKLLGLSDDYEFKGEKGTYF | |
| | IEFKKYKEFIKALGEHNLSQDDLNEIAKDITLIKDEIKLKKALAKYD | |
| | LNQNQIDSLSKLEFKDHLNISFKALKLVTPLMLEGKKYDEACNELNL | |
| | KVAINEDKKDFLPAFNETYYKDEVTNPVVLRAIKEYRKVLNALLKKY | |
| | GKVHKINIELAREVGKNHSQRAKIEKEQNENYKAKKDAELECEKLGL | |
| | KINSKNILKLRLFKEQKEFCAYSGEKIKISDLQDEKMLEIDAIYPYS | |
| | RSFDDSYMNKVLVFTKQNQEKLNQTPFEAFGNDSAKWQKIEVLAKNL | |
| | PTKKQKRILDKNYKDKEQKNFKDRNLNDTRYIARLVLNYTKDYLDFL | |
| | PLSDDENTKLNDTQKGSKVHVEAKSGMLTSALRHTWGFSAKDRNNHL | |
| | HHAIDAVIIAYANNSIVKAFSDFKKEQESNSAELYAKKISELDYKNK | |
| | RKFFEPFSGFRQKVLDKIDEIFVSKPERKKPSGALHEETFRKEEEFY | |
| | QSYGGKEGVLKALELGKIRKVNGKIVKNGDMFRVDIFKHKKTNKFYA | |
| | VPIYTMDFALKVLPNKAVARSKKGEIKDWILMDENYEFCFSLYKDSL | |
| | ILIQTKDMQEPEFVYYNAFTSSTVSLIVSKHDNKFETLSKNQKILFK | |
| | NANEKEVIAKSIGIQNLKVFEKYIVSALGEVTKAEFRQREDFKK | |
| Corynebacterium | MKYHVGIAVGTFSVGLAAIEVDDAGMPIKTLSLVSHIHDSGLDPDEI | SEQ ID NO: |
| diphtheria | KSAVTRLASSGIARRTRRLYRRKRRRLQQLDKFIQRQGWPVIELEDY | 6 |
| dCas9 | SDPLYPWKVRAELAASYIADEKERGEKLSVALRHIARHRGWRNPYAK | |
| (D8A, | VSSLYLPDGPSDAFKAIREEIKRASGQPVPETATVGQMVTLCELGTL | |
| H573A) | KLRGEGGVLSARLQQSDYAREIQEICRMQEIGQELYRKIIDVVFAAE | |
| | SPKGSASSRVGKDPLQPGKNRALKASDAFQRYRIAALIGNLRVRVDG | |
| | EKRILSVEEKNLVFDHLVNLTPKKEPEWVTIAEILGIDRGQLIGTAT | |
| | MTDDGERAGARPPTHDTNRSIVNSRIAPLVDWWKTASALEQHAMVKA | |
| | LSNAEVDDFDSPEGAKVQAFFADLDDDVHAKLDSLHLPVGRAAYSED | |
| | TLVRLTRRMLSDGVDLYTARLQEFGIEPSWTPPTPRIGEPVGNPAVD | |
| | RVLKTVSRWLESATKTWGAPERVIIEHVREGFVTEKRAREMDGDMRR | |
| | RAARNAKLFQEMQEKLNVQGKPSRADLWRYQSVQRQNCQCAYCGSPI | |
| | TFSNSEMDAIVPRAGQGSTNTRENLVAVCHRCNQSKGNTPFAIWAKN | |
| | TSIEGVSVKEAVERTRHWVTDTGMRSTDFKKFTKAVVERFQRATMDE | |
| | EIDARSMESVAWMANELRSRVAQHFASHGTTVRVYRGSLTAEARRAS | |
| | GISGKLKFFDGVGKSRLDRRHHAIDAAVIAFTSDYVAETLAVRSNLK | |
| | QSQAHRQEAPQWREFTGKDAEHRAAWRVWCQKMEKLSALLTEDLRDD | |
| | RVVVMSNVRLRLGNGSAHKETIGKLSKVKLSSQLSVSDIDKASSEAL | |
| | WCALTREPGFDPKEGLPANPERHIRVNGTHVYAGDNIGLFPVSAGSI | |
| | ALRGGYAELGSSFHHARVYKITSGKKPAFAMLRVYTIDLLPYRNQDL | |
| | FSVELKPQTMSMRQAEKKLRDALATGNAEYLGWLVVDDELVVDTSKI | |
| | ATDQVKAVEAELGTIRRWRVDGFFSPSKLRLRPLQMSKEGIKKESAP | |
| | ELSKIIDRPGWLPAVNKLFSDGNVTVVRRDSLGRVRLESTAHLPVTW | |
| | KVQ | |
| Eubacterium | MGYTVGLAIGVASVGVAVLDENDNIVEAVSNIFDEADTSNNKVRRTL | SEQ ID NO: |
| ventriosum | REGRRTKRRQKTRIEDFKQLWETSGYIIPHKLHLNIIELRNKGLTEL | 7 |
| dCas9 | LSLDELYCVLLSMLKHRGISYLEDADDGEKGNAYKKGLAFNEKQLKE | |
| (D8A, | KMPCEIQLERMKKYGKYHGEFIIEINDEKEYQSNVFTTKAYKKELEK | |
| H592A) | IFETQRCNGNKINTKFIKKYMEIYERKREYYIGPGNEKSRTDYGIYT | |
| | TRTDEEGNFIDEKNIFGKLIGKCSVYPEEYRASSASYTAQEFNLLND | |
| | LNNLKINNEKLTEFQKKEIVEIIKDASSVNMRKIIKKVIDEDIEQYS | |
| | GARIDKKGKEIYHTFEIYRKLKKELKTINVDIDSFTREELDKTMDIL | |
| | TLNTERESIVKAFDEQKFVYEENLIKKLIEFRKNNQRLFSGWHSFSY | |
| | KAMLQLIPVMYKEPKEQMQLLTEMNVFKSKKEKYVNYKYIPENEVVK | |
| | EIYNPVVVKSIRTTVKILNALIKKYGYPESVVIEMPRDKNSDDEKEK | |
| | IDMNQKKNQEEYEKILNKIYDEKGIEITNKDYKKQKKLVLKLKLWNE | |
| | QEGLCLYSGKKIAIEDLLNHPEFFEIDAIIPKSISLDDSRSNKVLVY | |
| | KTENSIKENDTPYHYLTRINGKWGFDEYKANVLELRRRGKIDDKKVN | |
| | NLLCMEDITKIDVVKGFINRNLNDTRYASRVVLNEMQSFFESRKYCN | |
| | TKVKVIRGSLTYQMRQDLHLKKNREESYSHHAVDAMLIAFSQKGYEA | |
| | YRKIQKDCYDFETGEILDKEKWNKYIDDDEFDDILYKERMNEIRKKI | |
| | IEAEEKVKYNYKIDKKCNRGLCNQTIYGTREKDGKIHKISSYNIYDD | |
| | KECNSLKKMINSGKGSDLLMYNNDPKTYRDMLKILETYSSEKNPFVA | |
| | YNKETGDYFRKYSKNHNGPKVEKVKYYSGQINSCIDISHKYGHAKNS | |
| | KKVVLVSLNPYRTDVYYDNDTGKYYLVGVKYNHIKCVGNKYVIDSET | |
| | YNELLRKEGVLNSDENLEDLNSKNITYKFSLYKNDIIQYEKGGEYYT | |
| | ERFLSRIKEQKNLIETKPINKPNFQRKNKKGEWENTRNQIALAKTKY | |
| | VGKLVTDVLGNCYIVNMEKFSLVVDK | |
| Streptococcus | MTNGKILGLAIGIASVGVGIIEAKTGKVVHANSRLFSAANAENNAER | SEQ ID NO: |
| pasteurianus | RGFRGSRRLNRRKKHRVKRVRDLFEKYGIVTDFRNLNLNPYELRVKG | 8 |
| dCas9 | LTEQLKNEELFAALRTISKRRGISYLDDAEDDSTGSTDYAKSIDENR | |
| (D10A, | RLLKNKTPGQIQLERLEKYGQLRGNFTVYDENGEAHRLINVESTSDY | |
| H599A) | EKEARKILETQADYNKKITAEFIDDYVEILTQKRKYYHGPGNEKSRT | |
| | DYGRFRTDGTTLENIFGILIGKCNFYPDEYRASKASYTAQEYNFLND | |
| | LNNLKVSTETGKLSTEQKESLVEFAKNTATLGPAKLLKEIAKILDCK | |
| | VDEIKGYREDDKGKPDLHTFEPYRKLKFNLESINIDDLSREVIDKLA | |
| | DILTLNTEREGIEDAIKRNLPNQFTEEQISEIIKVRKSQSTAFNKGW | |
| | HSFSAKLMNELIPELYATSDEQMTILTRLEKFKVNKKSSKNTKTIDE | |
| | KEVTDEIYNPVVAKSVRQTIKIINAAVKKYGDFDKIVIEMPRDKNAD | |
| | DEKKFIDKRNKENKKEKDDALKRAAYLYNSSDKLPDEVEHGAKQLET | |
| | KIRLWYQQGERCLYSGKPISIQELVHNSNNFEIDHILPLSLSFDDSL | |
| | ANKVLVYAWTNQEKGQKTPYQVIDSMDAAWSFREMKDYVLKQKGLGK | |
| | KKRDYLLTTENIDKIEVKKKFIERNLVDTRYASRVVLNSLQSALREL | |
| | GKDTKVSVVRGQFTSQLRRKWKIDKSRETYHHHAVDALIIAASSQLK | |
| | LWEKQDNPMFVDYGKNQVVDKQTGEILSVSDDEYKELVFQPPYQGFV | |
| | NTISSKGFEDEILFSYQVDSKYNRKVSDATIYSTRKAKIGKDKKEET | |
| | YVLGKIKDIYSQNGFDTFIKKYNKDKTQFLMYQKDSLTWENVIEVIL | |
| | RDYPTTKKSEDGKNDVKCNPFEEYRRENGLICKYSKKGKGTPIKSLK | |
| | YYDKKLGNCIDITPEESRNKVILQSINPWRADVYFNPETLKYELMGL | |
| | KYSDLSFEKGIGNYHISQEKYDAIKEKEGIGKKSEFKFTLYRNDLIL | |
| | IKDIASGEQEIYRFLSRTMPNVNHYVELKPYDKEKFDNVQELVEALG | |
| | EADKVGRCIKGLNKPNISIYKVRTDVLGNKYFVKKKGDKPKLDFKNN | |
| | KK | |
| Lactobacillus | MTDRISLGLAIGVASVGFSVLDIDKGKVIELGARLFNATVAAGNQDR | SEQ ID NO: |
| farciminis | RDMRGSRRLLNRNKQRRKDVSKLFQEYGLLDNFDRDNFHTAFNNNWN | 9 |
| dCas9 | PYELRVKGLTKQLNKEELADSLYQIIKRRGISYALKDADANEDGTDY | |
| (D10A, | SSSLKINSQELIEKTPAQIQLQRLNDYGKVRGKVIVGDDIDDQKVLL | |
| H611A) | NVFPTNAYEKEARQIIATQQQFYPEILTGEFVKEYCQILTRKRDYFV | |
| | GPGNEKSRTDYGIYKTDGRILDNLFEELIGHDKIYPDELRASAASYT | |
| | AQLFNVLNDLNNLRILSYEDGKLTQIDKEKIIAEIKNNTTVVNMLNV | |
| | IKKVTGCQKDDIKGFRLNEKDKPEISSMPIYRKAHKDFLKVGIDITD | |
| | WPIDFIDRLSFILTLNTENGEIRKQINKRLVPDFDELNDDLVQLIID | |
| | NKDSFDIKTNNKWHRFSLKTMNKLIPTMIERPVEQMTLLTEMGLIKK | |
| | DTKRFEGNKYLPYKEIANDIFNPVASKSVREALKIVNAVLKKYGHIE | |
| | YIVIEMPRDKNLDEERKQIADFQKKNKKIKDAAFNAFVKAVGSKKNV | |
| | KIALSKNRKLQMGIRLWHQQQGIDPYNGEEINANDLISNPDKFEIDA | |
| | IIPQSISFDDGINNKTLCFASMNQVKGQKTPYEFLNEGHGQGYAKFK | |
| | AIVSKNKNFSKAKRDNYLFEENVSNIETRKRFLSRNLVDTRYSSRVV | |
| | LNSLQEFFHEKSDDTKVTVIRGKFTSNMRKHWHIDKTRDTFHHHAID | |
| | ASIIAATPFLRMWKKGSTIFPTKINETSIDIETGEILDDKNFDKVMY | |
| | EEPYSGFISEIMNADDRIKFSHQVDKKMNRKVSDATIYSTRIGKLSK | |
| | DKKDAEYVVAKVKDIYSVDGYENFKKVYKKDKTKELLYKYDPRTFSE | |
| | LEKIVIDYPDKIEKVQINGKIKAVDISPFELYRRDNGMVRKYSKKTG | |
| | PAIKQLKYLDKKLGSHIDITPKNANNKHVVLQSLKPWRTDVYLNHET | |
| | GEYEIMGIKYSDLKFNKNEGYGIKKDKYLKIKNIEGVSENSEFMFSL | |
| | YRKDRIKVQDMETNESVELLFWSRNFSNKKYAELKPISQIDNDKVLP | |
| | IYGRGRLIKRLIPKNCKIWKVNTSILGDTYYLEKESNYPQKILD | |
| Sphaerochaeta | MSKKVSRRYEEQAQEICQRLGSRPYSIGLALGVGSIGVAVAAYDPIK | SEQ ID NO: |
| globosa | KQPSDLVFVSSRIFIPSTGAAERRQKRGQRNSLRHRANRLKFLWKLL | 10 |
| dCas9 | AERNLMLSYSEQDVPDPARLRFEDAVVRANPYELRLKGLNEQLTLSE | |
| (D30A, | LGYALYHIANHRGSSSVRTFLDEEKSSDDKKLEEQQAMTEQLAKEKG | |
| H635A) | ISTFIEVLTAFNINGLIGYRNSESVKSKGVPVPTRDIISNEIDVLLQ | |
| | TQKQFYQEILSDEYCDRIVSAILFENEKIVPEAGCCPYFPDEKKLPR | |
| | CHFLNEERRLWEAINNARIKMPMQEGAAKRYQSASFSDEQRHILFHI | |
| | ARSGTDITPKLVQKEFPALKTSIIVLQGKEKAIQKIAGFRFRRLEEK | |
| | SFWKRLSEEQKDDFFSAWTNTPDDKRLSKYLMKHLLLTENEVVDALK | |
| | TVSLIGDYGPIGKTATQLLMKHLEDGLTYTEALERGMETGEFQELSV | |
| | WEQQSLLPYYGQILTGSTQALMGKYWHSAFKEKRDSEGFFKPNTNSD | |
| | EEKYGRIANPVVHQTLNELRKLMNELITILGAKPQEITVELARELKV | |
| | GAEKREDIIKQQTKQEKEAVLAYSKYCEPNNLDKRYIERFRLLEDQA | |
| | FVCPYCLEHISVADIAAGRADVDAIFPRDDTADNSYGNKVVAHRQCN | |
| | DIKGKRTPYAAFSNTSAWGPIMHYLDETPGMWRKRRKFETNEEEYAK | |
| | YLQSKGFVSRFESDNSYIAKAAKEYLRCLFNPNNVTAVGSLKGMETS | |
| | ILRKAWNLQGIDDLLGSRHWSKDADTSPTMRKNRDDNRHHGLDAIVA | |
| | LYCSRSLVQMINTMSEQGKRAVEIEAMIPIPGYASEPNLSFEAQREL | |
| | FRKKILEFMDLHAFVSMKTDNDANGALLKDTVYSILGADTQGEDLVF | |
| | VVKKKIKDIGVKIGDYEEVASAIRGRITDKQPKWYPMEMKDKIEQLQ | |
| | SKNEAALQKYKESLVQAAAVLEESNRKLIESGKKPIQLSEKTISKKA | |
| | LELVGGYYYLISNNKRTKTFVVKEPSNEVKGFAFDTGSNLCLDFYHD | |
| | AQGKLCGEIIRKIQAMNPSYKPAYMKQGYSLYVRLYQGDVCELRASD | |
| | LTEAESNLAKTTHVRLPNAKPGRIFVIIITFTEMGSGYQIYFSNLAK | |
| | SKKGQDTSFTLTTIKNYDVRKVQLSSAGLVRYVSPLLVDKIEKDEVA | |
| | LCGE | |
| Azospirillum | MARPAFRAPRREHVNGWTPDPHRISKPFFILVSWHLLSRVVIDSSSG | SEQ ID NO: |
| (strain | CFPGTSRDHTDKFAEWECAVQPYRLSFALGINSIGWGLLNLDRQGKP | 11 |
| B510) | REIRALGSRIFSDGRDPQDKASLAVARRLARQMRRRRDRYLTRRTRL | |
| dCas9 | MGALVRFGLMPADPAARKRLEVAVDPYLARERATRERLEPFEIGRAL | |
| (D75A, | FHLNQRRGYKPVRTATKPDEEAGKVKEAVERLEAAIAAAGAPTLGAW | |
| H680A) | FAWRKTRGETLRARLAGKGKEAAYPFYPARRMLEAEFDTLWAEQARH | |
| | HPDLLTAEAREILRHRIFHQRPLKPPPVGRCTLYPDDGRAPRALPSA | |
| | QRLRLFQELASLRVIHLDLSERPLTPAERDRIVAFVQGRPPKAGRKP | |
| | GKVQKSVPFEKLRGLLELPPGTGFSLESDKRPELLGDETGARIAPAF | |
| | GPGWTALPLEEQDALVELLLTEAEPERAIAALTARWALDEATAAKLA | |
| | GATLPDFHGRYGRRAVAELLPVLERETRGDPDGRVRPIRLDEAVKLL | |
| | RGGKDHSDFSREGALLDALPYYGAVLERHVAFGTGNPADPEEKRVGR | |
| | VANPTVHIALNQLRHLVNAILARHGRPEEIVIELARDLKRSAEDRRR | |
| | EDKRQADNQKRNEERKRLILSLGERPTPRNLLKLRLWEEQGPVENRR | |
| | CPYSGETISMRMLLSEQVDIDAILPFSVSLDDSAANKVVCLREANRI | |
| | KRNRSPWEAFGHDSERWAGILARAEALPKNKRWRFAPDALEKLEGEG | |
| | GLRARHLNDTRHLSRLAVEYLRCVCPKVRVSPGRLTALLRRRWGIDA | |
| | ILAEADGPPPEVPAETLDPSPAEKNRADHRHHALDAVVIGCIDRSMV | |
| | QRVQLAAASAEREAAAREDNIRRVLEGFKEEPWDGFRAELERRARTI | |
| | VVSHRPEHGIGGALHKETAYGPVDPPEEGFNLVVRKPIDGLSKDEIN | |
| | SVRDPRLRRALIDRLAIRRRDANDPATALAKAAEDLAAQPASRGIRR | |
| | VRVLKKESNPIRVEHGGNPSGPRSGGPFHKLLLAGEVHHVDVALRAD | |
| | GRRWVGHWVTLFEAHGGRGADGAAAPPRLGDGERFLMRLHKGDCLKL | |
| | EHKGRVRVMQVVKLEPSSNSVVVVEPHQVKTDRSKHVKISCDQLRAR | |
| | GARRVTVDPLGRVRVHAPGARVGIGGDAGRTAMEPAEDIS | |
| Gluconacetobacter | MIDESLTFGIALGIGSCGWAVLRRPSAFGRKGVIEGMGSWCFDVPET | SEQ ID NO: |
| diazotrophicus | SKERTPTNQIRRSNRLLRRVIRRRRNRMAAIRRLLHAAGLLPSTDSD | 12 |
| dCas9 | ALKRPGHDPWELRARGLDKPLKPVEFAVVLGHIAKRRGFKSAAKRKA | |
| (D11A, | TNISSDDKKMLTALEATRERLGRYRTVGEMFARDPDFASRRRNREGK | |
| H587A) | YDRTTARDDLEHEVHALFAAQRRLGQGFASPELEEAFTASAFHQRPM | |
| | QDSERLVGFCPFERTEKRAAKLTPSFERFRLLARLLNLRITTPDGER | |
| | PLTVDEIALVTRDLGKTAKLSIKRVRTLIGLEDNQRFTTIRPEDEDR | |
| | DIVARTGGAMTGTATLRKALGEALWTDMQERPEQLDAIVQVLSFFEA | |
| | NETITEKLREIGLTLAVLDVLLTALDAGVFAKFKGAAHISTKAARNL | |
| | LPHLEQGRRYDEACTMAGYDHAASRLSHHGQIVAKTQFNALVTEIGE | |
| | SIANPIARKALIEGLKQIWAMRNHWGLPGSIHVELARDVGNSIEKRR | |
| | EIEKHIEKNTALRARERREVHDLLDLEDVNGDTLLRYRLWKEQGGKC | |
| | LYTGKAIHIRQIAATDNSVQVDAILPWSRFGDDSENNKTLCLASANQ | |
| | QKKRSTPYEWLSGQTGDAWNAFVQRIETNKELRGFKKRNYLLKNAKE | |
| | AEEKFRSRNLNDTRYAARLFAEAVKLLYAFGERQEKGGNRRVFTRPG | |
| | ALTAALRQAWGVESLKKQDGKRINDDRHHALDALTVAAVDEAEIQRL | |
| | TKSFHEWEQQGLGRPLRRVEPPWESFRADVEATYPEVFVARPERRRA | |
| | RGEGHAATIRQVKERECTPIVFERKAVSSLKEADLERIKDGERNEAI | |
| | VEAIRSWIATGRPADAPPRSPRGDIITKIRLATTIKAAVPVRGGTAG | |
| | RGEMVRADVFSKPNRRGKDEWYLVPVYPHQIMNRKAWPKPPMRSIVA | |
| | NKDEDEWTEVGPEHQFRFSLYPRSNIEIIRPSGEVIEGYFVGLHRNT | |
| | GALIPTPVGPDSYVIA | |
| Neisseria | MAAFKPNPMNYILGLAIGIASVGWAIVEIDEEENPIRLIDLGVRVFE | SEQ ID NO: |
| cinerea | RAEVPKTGDSLAAARRLARSVRRLTRRRAHRLLRARRLLKREGVLQA | 13 |
| dCas9 | ADFDENGLIKSLPNTPWQLRAAALDRKLTPLEWSAVLLHLIKHRGYL | |
| (D16A, | SQRKNEGETADKELGALLKGVADNTHALQTGDFRTPAELALNKFEKE | |
| H588A) | SGHIRNQRGDYSHTFNRKDLQAELNLLFEKQKEFGNPHVSDGLKEGI | |
| | ETLLMTQRPALSGDAVQKMLGHCTFEPTEPKAAKNTYTAERFVWLTK | |
| | LNNLRILEQGSERPLTDTERATLMDEPYRKSKLTYAQARKLLDLDDT | |
| | AFFKGLRYGKDNAEASTLMEMKAYHAISRALEKEGLKDKKSPLNLSP | |
| | ELQDEIGTAFSLFKTDEDITGRLKDRVQPEILEALLKHISFDKFVQI | |
| | SLKALRRIVPLMEQGNRYDEACTEIYGDHYGKKNTEEKIYLPPIPAD | |
| | EIRNPVVLRALSQARKVINGVVRRYGSPARIHIETAREVGKSFKDRK | |
| | EIEKRQEENRKDREKSAAKFREYFPNFVGEPKSKDILKLRLYEQQHG | |
| | KCLYSGKEINLGRLNEKGYVEIDAALPFSRTWDDSFNNKVLALGSEN | |
| | QNKGNQTPYEYFNGKDNSREWQEFKARVETSRFPRSKKQRILLQKFD | |
| | EDGFKERNLNDTRYINRFLCQFVADHMLLTGKGKRRVFASNGQITNL | |
| | LRGFWGLRKVRAENDRHHALDAVVVACSTIAMQQKITRFVRYKEMNA | |
| | FDGKTIDKETGEVLHQKAHFPQPWEFFAQEVMIRVFGKPDGKPEFEE | |
| | ADTPEKLRTLLAEKLSSRPEAVHKYVTPLFISRAPNRKMSGQGHMET | |
| | VKSAKRLDEGISVLRVPLTQLKLKDLEKMVNREREPKLYEALKARLE | |
| | AHKDDPAKAFAEPFYKYDKAGNRTQQVKAVRVEQVQKTGVWVHNHNG | |
| | IADNATIVRVDVFEKGGKYYLVPIYSWQVAKGILPDRAVVQGKDEED | |
| | WTVMDDSFEFKFVLYANDLIKLTAKKNEFLGYFVSLNRATGAIDIRT | |
| | HDTDSTKGKNGIFQSVGVKTALSFQKYQIDELGKEIRPCRLKKRPPV | |
| | R | |
| Roseburia | MRENGSDERRRNMDEKMDYRIGLAIGIASVGWAVLQNNSDDEPVRIV | SEQ ID NO: |
| intestinalis | DLGVRIFDTAEIPKTGESLAGPRRAARTTRRRLRRRKHRLDRIKWLF | 14 |
| dCas9 | ENQGLINIDDFLKRYNMAGLPDVYQLRYEALDRKLTDEELAQVLLHI | |
| (D24A, | AKHRGFRSTRKAETAAKENGAVLKATDENQKRMQEKGYRTVGEMIYL | |
| H628A) | DEAFRTGCSWSEKGYILTPRNKAENYQHTMLRAMLVEEVKEIFSSQR | |
| | RLGNEKATEELEEKYLEIMTSQRSFDLGPGMQPDGKPSPYAMEGFSD | |
| | RVGKCTFLGDQGELRGAKGTYTAEYFVALQKINHTKLVNQDGETRNE | |
| | TEEERRALTLLLFTQKEVKYAAVRKKLGLPEDILFYNLNYKKAATKE | |
| | EQQKENQNTEKAKFIGMPYYHDYKKCLEERVKYLTENEVRDLFDEIG | |
| | MILTCYKNDDSRTERLAKLGLVPIEMEGLLAYTPTKFQHLSMKAMRN | |
| | IIPFLEKGMTYDKACEEAGYDFKADSKGTKQKLLIGENVNQTINEIT | |
| | NPVVKRSVSQTVKVINAIIRTYGSPQAINIELAREMSKTFEERRKIK | |
| | GDMEKRQKNNEDVKKQIQELGKLSPTGQDILKYRLWQEQQGICMYSG | |
| | KTIPLEELFKPGYDIDAILPYSITFDDSFRNKVLVTSQENRQKGNRT | |
| | PYEYMGNDEQRWNEFETRVKTTIRDYKKQQKLLKKHFSEEERSEFKE | |
| | RNLTDTKYITTVIYNMIRQNLEMAPLNRPEKKKQVRAVNGAITAYLR | |
| | KRWGLPQKNRETDTHHAMDAVVIACCTDGMIQKISRYTKVRERCYSK | |
| | GTEFVDAETGEIFRPEDYSRAEWDEIFGVHIPKPWETFRAELDVRMG | |
| | DDPKGFLDTHSDVALELDYPEYIYENLRPIFVSRMPNHKVTGAAHAD | |
| | TIRSPRHFKDEGIVLTKTALTDLKLDKDGEIDGYYNPQSDLLLYEAL | |
| | KKQLLLYGNDAKKAFAQDFHKPKADGTEGPVVRKVKIQKKQTMGVFV | |
| | DSGNGIAENGGMVRIDVFRVNGKYYFVPVYTADVVKKVLPNRASTAH | |
| | KPYGEWKVMEDKDFLFSLYSRDLIHIKSKKDIPIKMVNGGMEGIKET | |
| | YAYYIGADISAANIQGIAHDSRYKFRGLGIQSLDVLEKCQIDVLGHV | |
| | SVVRSEKRMGFS | |
| Parvibaculum | MERIFGFAIGTTSIGFSVIDYSSTQSAGNIQRLGVRIFPEARDPDGT | SEQ ID NO: |
| lavamentivorans | PLNQQRRQKRMMRRQLRRRRIRRKALNETLHEAGFLPAYGSADWPVV | 15 |
| dCas9 | MADEPYELRRRGLEEGLSAYEFGRAIYHLAQHRHFKGRELEESDTPD | |
| (D8A, | PDVDDEKEAANERAATLKALKNEQTTLGAWLARRPPSDRKRGIHAHR | |
| H601A) | NVVAEEFERLWEVQSKFHPALKSEEMRARISDTIFAQRPVFWRKNTL | |
| | GECRFMPGEPLCPKGSWLSQQRRMLEKLNNLAIAGGNARPLDAEERD | |
| | AILSKLQQQASMSWPGVRSALKALYKQRGEPGAEKSLKFNLELGGES | |
| | KLLGNALEAKLADMFGPDWPAHPRKQEIRHAVHERLWAADYGETPDK | |
| | KRVIILSEKDRKAHREAAANSFVADFGITGEQAAQLQALKLPTGWEP | |
| | YSIPALNLFLAELEKGERFGALVNGPDWEGWRRINFPHRNQPTGEIL | |
| | DKLPSPASKEERERISQLRNPTVVRTQNELRKVVNNLIGLYGKPDRI | |
| | RIEVGRDVGKSKREREEIQSGIRRNEKQRKKATEDLIKNGIANPSRD | |
| | DVEKWILWKEGQERCPYTGDQIGFNALFREGRYEVEAIWPRSRSFDN | |
| | SPRNKTLCRKDVNIEKGNRMPFEAFGHDEDRWSAIQIRLQGMVSAKG | |
| | GTGMSPGKVKRFLAKTMPEDFAARQLNDTRYAAKQILAQLKRLWPDM | |
| | GPEAPVKVEAVTGQVTAQLRKLWTLNNILADDGEKTRADHRHHAIDA | |
| | LTVACTHPGMINKLSRYWQLRDDPRAEKPALTPPWDTIRADAEKAVS | |
| | EIVVSHRVRKKVSGPLHKETTYGDTGTDIKTKSGTYRQFVTRKKIES | |
| | LSKGELDEIRDPRIKEIVAAHVAGRGGDPKKAFPPYPCVSPGGPEIR | |
| | KVRLTSKQQLNLMAQTGNGYADLGSNHHIAIYRLPDGKADFEIVSLF | |
| | DASRRLAQRNPIVQRTRADGASFVMSLAAGEAIMIPEGSKKGIWIVQ | |
| | GVWASGQVVLERDTDADHSTTTRPMPNPILKDDAKKVSIDPIGRVRP | |
| | SND | |
| Nitratifractor | MKKILGVALGITSFGYAILQETGKDLYRCLDNSVVMRNNPYDEKSGE | SEQ ID NO: |
| salsuginis | SSQSIRSTQKSMRRLIEKRKKRIRCVAQTMERYGILDYSETMKINDP | 16 |
| (strain | KNNPIKNRWQLRAVDAWKRPLSPQELFAIFAHMAKHRGYKSIATEDL | |
| DSM | IYELELELGLNDPEKESEKKADERRQVYNALRHLEELRKKYGGETIA | |
| 16511) | QTIHRAVEAGDLRSYRNHDDYEKMIRREDIEEEIEKVLLRQAELGAL | |
| dCas9 | GLPEEQVSELIDELKACITDQEMPTIDESLFGKCTFYKDELAAPAYS | |
| (D8A, | YLYDLYRLYKKLADLNIDGYEVTQEDREKVIEWVEKKIAQGKNLKKI | |
| H611A) | THKDLRKILGLAPEQKIFGVEDERIVKGKKEPRTFVPFFFLADIAKF | |
| | KELFASIQKHPDALQIFRELAEILQRSKTPQEALDRLRALMAGKGID | |
| | TDDRELLELFKNKRSGTRELSHRYILEALPLFLEGYDEKEVQRILGF | |
| | DDREDYSRYPKSLRHLHLREGNLFEKEENPINNHAVKSLASWALGLI | |
| | ADLSWRYGPFDEIILETTRDALPEKIRKEIDKAMREREKALDKIIGK | |
| | YKKEFPSIDKRLARKIQLWERQKGLDLYSGKVINLSQLLDGSADIEA | |
| | IVPQSLGGLSTDYNTIVTLKSVNAAKGNRLPGDWLAGNPDYRERIGM | |
| | LSEKGLIDWKKRKNLLAQSLDEIYTENTHSKGIRATSYLEALVAQVL | |
| | KRYYPFPDPELRKNGIGVRMIPGKVTSKTRSLLGIKSKSRETNFHHA | |
| | EDALILSTLTRGWQNRLHRMLRDNYGKSEAELKELWKKYMPHIEGLT | |
| | LADYIDEAFRRFMSKGEESLFYRDMEDTIRSISYWVDKKPLSASSHK | |
| | ETVYSSRHEVPTLRKNILEAFDSLNVIKDRHKLTTEEFMKRYDKEIR | |
| | QKLWLHRIGNINDESYRAVEERATQIAQILTRYQLMDAQNDKEIDEK | |
| | FQQALKELITSPIEVTGKLLRKMRFVYDKLNAMQIDRGLVETDKNML | |
| | GIHISKGPNEKLIFRRMDVNNAHELQKERSGILCYLNEMLFIFNKKG | |
| | LIHYGCLRSYLEKGQGSKYIALFNPRFPANPKAQPSKFTSDSKIKQV | |
| | GIGSATGIIKAHLDLDGHVRSYEVFGTLPEGSIEWFKEESGYGRVED | |
| | DPHH | |
| Campylobacter | MKILGFAIGINSIGWAFVENDELKDCGVRIFTEAENPSNKESLALPR | SEQ ID NO: |
| lari | RNARSSRRRLKRRKARLVAIKRILAKELKLNFKDYVAADGELPKAYE | 17 |
| dCas9 | GKLTSIYELRYKALIQQLETKDLARVILHIAKHRGYMNKNEKKSNDT | |
| (D7A, | KKGKILSALKNNALKLENYQSVGEYFYKEFFQKYKENTKNFIKIRNT | |
| H567A) | KDNYNNCVLSSDLEKELKLILEKQKEFGYNYSEDFINEILKVAFFQR | |
| | PLKDFSHLVGACTFFEEEKRACKNSYSAWEFVALTKIINEIKSLEKI | |
| | SGEIVPTQTINEILNLILDKGSITYKKFRSCINLHESISFKSLKYDK | |
| | ENAENAKLIDFRKLVEFKKALGVHSLSRQELDQISTHITLIKDNVKL | |
| | KTVLEKYNLSNEQINNLLEIEFNDYINLSFKALGMILPLMREGKRYY | |
| | EACEITNLKPKTVDEKEDFLPAFCDSIFAHELSNPVVNRAISEYRKV | |
| | LNALLKKYGKVHKIHLELARDVGLSKKAREKIEDQQKNNKAINDWAL | |
| | KECENIGIKASAKNILKLKLWKEQKEICIYSGNKISIEHLKDEKALE | |
| | VDAIYPYSRSFDDSFINKVLVFTKENQEKLNKTPFEAFGKNIEKWSK | |
| | IQTLAQNLPYKKKNKILDENFKDKQQQDFISRNLNDTRYITTLIAKY | |
| | TKEYLNFLPLSENENTNLKSGEKGSKIHIQTISGMLTSVLRHTWGED | |
| | KKDRNNHLHHALDAIIVAYSTSSIIKAFSDFKKNQELLKARFYAKEL | |
| | TSDNYKHQVKFFEPFKGFREKILSKIDEIFVSTPPRKRARGALHEET | |
| | FYSEDKMIKKYNSKEGLQIALSCGRVRKIGTKYVENDTMVRVDIFKK | |
| | QNKFYAIPIYVMDFALGILPNKIVITGKDKNNNPKQWQTIDESYEFC | |
| | FSLYKNDLILLQKKNMQEPEFAYYNSFDIDRSRIKIKKHDNKFENLT | |
| | SNQKLLFTNKDKGKNRTGIQNLKIFEKYIVTPLGDKIKADFQPRENI | |
| | SLKTSKKHGL | |
| Streptococcus | MTKPYSIGLAIGTNSVGWAVTTDNYKVPSKKMKVLGNTSKKYIKKNL | SEQ ID NO: |
| thermophilus | LGVLLFDSGITAEGRRLKRTARRRYTRRRNRILYLQEIFSTEMATLD | 18 |
| (strain | DAFFQRLDDSFLVPDDKRDSKYPIFGNLVEEKAYHDEFPTIYHLRKY | |
| LMD-9) | LADSTKKADLRLVYLALAHMIKYRGHFLIEGEFNSKNNDIQKNFQDF | |
| dCas9 | LDTYNAIFESDLSLENSKQLEEIVKDKISKLEKKDRILKLFPGEKNS | |
| (D10A, | GIFSEFLKLIVGNQADFRKCFNLDEKASLHFSKESYDEDLETLLGYI | |
| H847A) | GDDYSDVFLKAKKLYDAILLSGELTVTDNETEAPLSSAMIKRYNEHK | |
| | EDLALLKEYIRNISLKTYNEVFKDDTKNGYAGYIDGKTNQEDFYVYL | |
| | KKLLAEFEGADYFLEKIDREDFLRKQRTFDNGSIPYQIHLQEMRAIL | |
| | DKQAKFYPFLAKNKERIEKILTFRIPYYVGPLARGNSDFAWSIRKRN | |
| | EKITPWNFEDVIDKESSAEAFINRMTSFDLYLPEEKVLPKHSLLYET | |
| | FNVYNELTKVRFIAESMRDYQFLDSKQKKDIVRLYFKDKRKVTDKDI | |
| | IEYLHAIYGYDGIELKGIEKQFNSSLSTYHDLLNIINDKEFLDDSSN | |
| | EAIIEEIIHILTIFEDREMIKQRLSKFENIFDKSVLKKLSRRHYTGW | |
| | GKLSAKLINGIRDEKSGNTILDYLIDDGISNRNFMQLIHDDALSFKK | |
| | KIQKAQIIGDEDKGNIKEVVKSLPGSPAIKKGILQSIKIVDELVKVM | |
| | GGRKPESIVVEMARENQYTNQGKSNSQQRLKRLEKSLKELGSKILKE | |
| | NIPAKLSKIDNNALQNDRLYLYYLQNGKDMYTGDDLDIDRLSNYDID | |
| | AIIPQAFLKDNSIDNKVLVSSASNRGKSDDVPSLEVVKKRKTFWYQL | |
| | LKSKLISQRKFDNLTKAERGGLSPEDKAGFIQRQLVETRQITKHVAR | |
| | LLDEKFNNKKDENNRAVRTVKIITLKSTLVSQFRKDFELYKVREIND | |
| | FHHAHDAYLNAVVASALLKKYPKLEPEFVYGDYPKYNSFRERKSATE | |
| | KVYFYSNIMNIFKKSISLADGRVIERPLIEVNEETGESVWNKESDLA | |
| | TVRRVLSYPQVNVVKKVEEQNHGLDRGKPKGLFNANLSSKPKPNSNE | |
| | NLVGAKEYLDPKKYGGYAGISNSFTVLVKGTIEKGAKKKITNVLEFQ | |
| | GISILDRINYRKDKLNFLLEKGYKDIELIIELPKYSLFELSDGSRRM | |
| | LASILSINNKRGEIHKGNQIFLSQKFVKLLYHAKRISNTINENHRKY | |
| | VENHKKEFEELFYYILEFNENYVGAKKNGKLLNSAFQSWQNHSIDEL | |
| | CSSFIGPTGSERKGLFELTSRGSAADFEFLGVKIPRYRDYTPSSLLK | |
| | DATLIHQSVTGLYETRIDLAKLGEG | |

### Cas9 Fusion Proteins

The CRISPR/Cas9-based system may include a fusion molecule (e.g., DNMT3A-DNMT3L(3A3L)-dCas9-KRAB). The fusion molecule may comprise at least one DNA binding protein (e.g., dCas9), and at least one modulator of gene expression (e.g., KRAB, DNMT3A, DNMT3L, DNMT3A-DNMT3L fusion peptide). In some embodiments, the modulator of gene expression is chosen from a repressor of gene expression (e.g. KRAB), an activator of gene expression, or a modulator of epigenetic modification (e.g. DNMT3A, DNMT3L, DNMT3A-DNMT3L fusion peptide) or any combination thereof. Different modulators of gene expression are known in the art, see, e.g., Thakore et al., Nat Methods. 2016; 13: 127-37, incorporated by reference herein in its entirety.

### Repressors of Gene Expression

In some embodiments, the modulator of gene expression comprises a repressor of gene expression. The repressor may be any known repressor of gene expression, for example, a repressor chosen from Kruppel associated box (KRAB) domain, mSin3 interaction domain (SID), MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRTM28), Nuclear receptor co-repressor 1, Nuclear receptor co-repressor 2, or fragment or fusion thereof.

### Kruppel Associated Box (KRAB)

The KRAB domain is a type of transcriptional repression domains present in the N-terminal part of many zinc finger protein-based transcription factors. The KRAB domain functions as a transcriptional repressor when tethered to a target DNA by a DNA-binding domain. The KRAB domain is enriched in charged amino acids and can be divided into sub-domains A and B. The KRAB A and B sub-domains can be separated by variable spacer segments and many KRAB proteins contain only the A sub-domain. A sequence of 45 amino acids in the KRAB A sub-domain has been shown to be important for transcriptional repression. The B sub-domain does not repress transcription by itself but does potentiate the repression exerted by the KRAB A sub-domain. The KRAB domain recruits corepressors KAP1 (KRAB-associated protein-1, also known as transcription intermediary factor 1 beta, KRAB-A interacting protein and tripartite motif protein 28) and heterochromatin protein 1 (HP1), as well as other chromatin modulating proteins, leading to transcriptional repression through heterochromatin formation. In one embodiment, the methods and compositions disclosed herein include a fusion molecule comprising a dCas9 molecule fused to a KRAB domain or fragment thereof. In one embodiment, the KRAB domain or fragment thereof is fused to the N-terminus of the dCas9 molecule. In one embodiment, the KRAB domain or fragment thereof is fused to the C-terminus of the dCas9 molecule. In one embodiment, the KRAB domain or fragment thereof is fused to both the N-terminus and the C-terminus of the dCas9 molecule. In one embodiment, the fusion molecule comprises a KRAB domain comprising the sequence of SEQ ID NO: 22, a sequence substantially identical (e.g., at least 80%, 85%, 90%, 92%, 95%, 97%, 98%, 99% or higher identical) to SEQ ID NO: 22, or a sequence having one, two, three, four, five or more changes, e.g., amino acid substitutions, insertions, or deletions, relative to SEQ ID NO: 22, or any fragment thereof.

### Exemplary KRAB Domain Sequence

(SEQ ID NO: 22)
RTLVTFKDVFVDFTREEWKLLDTAQQIVYRNVMLENYKNLVSLGYQLTKP
DVILRLEKGEEP

mSin3 Interaction Domain (SID)

The mSin3 interaction domain (SID) is an interaction domain that is present on several transcription repressor proteins. It interacts with the paired amphipathic alpha-helix 2 (PAH2) domain of mSin3, a transcriptional repressor domain that is attached to transcription repressor proteins such as the mSin3 A corepressor. In one embodiment, the methods and compositions disclosed herein include a fusion molecule comprising a dCas9 molecule fused to an mSin3 interaction domain or fragment thereof. In one embodiment, the methods and compositions disclosed herein include a fusion molecule comprising a dCas9 molecule fused to four concatenated mSin3 interaction domains (SID4X). In one embodiment, the four concatenated mSin3 interaction domains (SID4X) are fused to the C-terminus of the dCas9 molecule.

### MAX-Interacting Protein 1 (MXI1)

Mxi1 is a repressor of MYC. Mxi1 antagonizes MYC transcriptional activity possibly by competing for binding to MYC-associated factor X (MAX), which binds to MYC and is required for MYC to function. In one embodiment, the methods and compositions disclosed herein include a fusion molecule comprising a dCas9 molecule fused to Mxi1 or fragment thereof. In one embodiment, Mxi1 is fused to the C-terminus of the dCas9 molecule.

### Activators of Gene Expression

In some embodiments, the modulator of gene expression comprises a activator of gene expression. The activator may be any known activator of gene expression, for example, a VP16 activation domain, a VP64 activation domain, a p65 activation domain, an Epstein-Barr virus R transactivator Rta molecule, or fragment thereof. Activations that can be used with a dCas9 molecule are known in the art. See, e.g., Chavez et al., Nat Methods. (2016) 13: 563-67, incorporated by reference herein in its entirety.

### VP16, VP64, VP160

VP16 is a viral protein sequence of 16 amino acids that recruits transcriptional activators to promoters and enhancers. VP64 is a transcription activator comprising four copies of VP 16, e.g., a molecule comprising four tandem copies of VP16 connected by Gly-Ser linkers. VP160 is a transcription activator comprising 10 copies of VP16. In one embodiment, the methods and compositions disclosed herein include a fusion molecule comprising a dCas9 molecule fused to 1, 2, 3, 4, 5, 6, 7, 8, 9, 10, or more copies of VP16. In one embodiment, the methods and compositions disclosed herein include a fusion molecule comprising a dCas9 molecule fused to VP64. In one embodiment, the methods and compositions disclosed herein include a fusion molecule comprising a dCas9 molecule fused to VP 160. In one embodiment, VP64 is fused to the C-terminus, the N-terminus, or both the N-terminus and the C-terminus of the dCas9 molecule.

p65 Activation Domain (p65AD)

p65AD is the principal transactivation domain of the 65 kDa polypeptide of the nuclear form of the F-κB transcription factor. An exemplary sequence of human transcription factor p65 is available at the Uniprot database under accession number Q04206. In one embodiment, the methods and compositions disclosed herein include a fusion molecule comprising a dCas9 molecule fused to p65 or fragment thereof, e.g., p65AD.

### Epstein-Barr Virus (EBV) R Transactivator (Rta)

Rta, an immediate-early protein of EBV, is a transcriptional activator that induces lytic gene expression and triggers virus reactivation. In one embodiment, the methods and compositions disclosed herein include a fusion molecule comprising a dCas9 molecule fused to Rta or fragment thereof.

VP64, p65, Rta Fusions

In one embodiment, the methods and compositions disclosed herein include a fusion molecule comprising a dCas9 molecule fused to VP64, p65, Rta, or any combination thereof. The tripartite activator VP64-p65-Rta (also known as VPR), in which the three transcription activation domains are fused using short amino acid linkers, can effectively up-regulate target gene expression when fused to a dCas9 molecule. In one embodiment, the methods and compositions disclosed herein include a fusion molecule comprising a dCas9 molecule fused to VPR.

### Synergistic Activation Mediators (SAM)

In one embodiment, the methods and compositions disclosed herein include a CRISPR-Cas system that comprises three components: (1) a dCas9-VP64 fusion, (2) a gRNA incorporating two MS2 RNA aptamers at the tetraloop and stem-loop, and (3) the MS2-P65-HSF1 activation helper protein. This system, named Synergistic Activation Mediators (SAM), brings together three activation domains—VP64, P65 and HSF1 and has been described in Konermann et al., Nature. 2015; 517:583-8, incorporated by reference herein in its entirety.

### Ldbl Self-Association Domain

In one embodiment, the methods and compositions disclosed herein include a fusion molecule comprising a dCas9 molecule fused to Ldbl self-association domain. Ldbl self-association domain recruits enhancer-associated endogenous Ldbl.

### Modulators of Epigenetic Modification

In one embodiment, the methods and compositions disclosed herein include a fusion molecule comprising a dCas9 molecule fused to a modulator of gene expression. In some embodiments, the modulator of gene expression comprises a modulator of epigenetic modification. In one embodiment, the fusion molecule modulates target gene expression via epigenetic modification, e.g., via histone acetylation or methylation, or DNA methylation, at a regulatory element of target gene, e.g., a promoter, enhancer or transcription start site. The modulator may be any known modulator of epigenetic modification, e.g., a histone acetyltransferase (e.g., p300 catalytic domain), a histone deacetylase, a histone methyltransferase (e.g., SUV39H1 or G9a (EHMT2)), a histone demethylase (e.g., LSD1), a DNA methyltransferase (e.g., DNMT3a or DNMT3a-DNMT3L), a DNA demethylase (e.g., TET1 catalytic domain or TDG), or fragment thereof.

### Histone Modification Activity

In some embodiments, the modulator of epigenetic modification may have histone modification activity. Histone modification activity may include but are not limited to histone deacetylase, histone acetyltransferase, histone demethylase, or histone methyltransferase activity.

In some embodiments, the modulator of epigenetic modification may have histone acetyltransferase activity. The histone acetyltransferase may be p300 or CREB-binding protein (CBP) protein, or fragments thereof. In some embodiments, the methods and compositions disclosed herein include a fusion molecule comprising a dCas9 molecule fused to acetyltransferase p300 or fragment thereof, e.g., the catalytic core of p300. In some embodiments, the methods and compositions disclosed herein include a fusion molecule comprising a dCas9 molecule fused to CREB-binding protein (CBP) protein or fragment thereof.

In some embodiments, the modulator of epigenetic modification may have histone demethylase activity. For example, the modulator of epigenetic modification may include an enzyme that removes methyl (CH3−) groups from nucleic acids or proteins (e.g., histones). In some embodiments, the methods and compositions disclosed herein include a fusion molecule comprising a dCas9 molecule fused to Lys-specific histone demethylase 1 (LSD1) or fragment thereof.

In some embodiments, the modulator of epigenetic modification may have histone methyltransferase activity. In some embodiments, the methods and compositions disclosed herein include a fusion molecule comprising a dCas9 molecule fused to SUV39H1 or fragment thereof. In some embodiments, the methods and compositions disclosed herein include a fusion molecule comprising a dCas9 molecule fused to G9a (EHMT2) or fragment thereof.

### DNA Demethylase Activity

In some embodiments, the modulator of epigenetic modification may have DNA demethylase activity. For example, the modulator of epigenetic modification may covert the methyl group to hydroxymethylcytosine as a mechanism for demethylating DNA. In some embodiments, the methods and compositions disclosed herein include a fusion molecule comprising a dCas9 molecule fused to Ten-eleven translocation methylcytosine dioxygenase 1 (TET1) or fragment thereof. In some embodiments, the methods and compositions disclosed herein include a fusion molecule comprising a dCas9 molecule fused to thymine DNA glycosylase (TDG) or fragment thereof.

### DNA Methylase Activity

In some embodiments, the modulator of epigenetic modification may have DNA methylase activity. For example, the modulator of epigenetic modification may have methylase activity which involves transferring a methyl group to DNA, RNA, proteins, small molecules, cytosine or adenine. In some embodiments, the methods and compositions disclosed herein include a fusion molecule comprising a dCas9 molecule fused to DNMT3A or fragment thereof. In some embodiments, the methods and compositions disclosed herein include a fusion molecule comprising a dCas9 molecule fused to DNMT3L or fragment thereof. In some embodiments, the methods and compositions disclosed herein include a fusion molecule comprising a dCas9 molecule fused to DNMT3A and DNMT3L or fragments thereof. In some embodiments, the methods and compositions disclosed herein include a fusion molecule comprising a dCas9 molecule fused to DNMT3A-DNMT3L fusion peptide.

DNMT3A
(SEQ ID NO: 23)
MNHDQEFDPPKVYPPVPAEKRKPIRVLSLFDGIATGLLVLKDLGIQVDRYIASEVCEDSITVGMVRHQ
GKIMYVGDVRSVTQKHIQEWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWGNLPGMNRPLASTVNDKLE
LQECLEHGRIAKFSKVRTITTRSNSIKQGKDQHFPVFMNEKEDILWCTEMERVFGFPVHYTDVSNMSR
LARQRLLGRSWSVPVIRHLFAPLKEYFACV
DNMT3L
(SEQ ID NO: 24)
MGPMEIYKTVSAWKRQPVRVLSLFRNIDKVLKSLGFLESGSGSGGGTLKYVEDVTNVVRRDVEKWGPF
DLVYGSTQPLGSSCDRCPGWYMFQFHRILQYALPRQESQRPFFWIFMDNLLLTEDDQETTTRFLQTEA
VTLQDVRGRDYQNAMRVWSNIPGLKSKHAPLTPKEEEYLQAQVRSRSKLDAPKVDLLVKNCLLPLREY
FKYFSQNSLPL
DNMT3A-DNMT3L fusion peptide
(SEQ ID NO: 96)
MNHDQEFDPPKVYPPVPAEKRKPIRVLSLFDGIATGLLVLKDLGIQVDRYIASEVCEDSITVGMVRHQ
GKIMYVGDVRSVTQKHIQEWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWGNLPGMNRPLASTVNDKLE
LQECLEHGRIAKFSKVRTITTRSNSIKQGKDQHFPVFMNEKEDILWCTEMERVFGFPVHYTDVSNMSR
LARQRLLGRSWSVPVIRHLFAPLKEYFACVSSGNSNANSRGPSFSSGLVPLSLRGSHMGPMEIYKTVS
AWKRQPVRVLSLFRNIDKVLKSLGFLESGSGSGGGTLKYVEDVTNVVRRDVEKWGPFDLVYGSTQPLG
SSCDRCPGWYMFQFHRILQYALPRQESQRPFFWIFMDNLLLTEDDQETTTRFLQTEAVTLQDVRGRDY
QNAMRVWSNIPGLKSKHAPLTPKEEEYLQAQVRSRSKLDAPKVDLLVKNCLLPLREYFKYFSQNSLPL

In one embodiment, the Cas9 fusion protein also comprises a nuclear localization sequence (NLS), e.g., a LS fused to the N-terminus and/or C-terminus of Cas9.

Nuclear localization sequences are known in the art. In one embodiment, the NLS comprises the amino acid sequence of SEQ ID NO: 25 or 26, a sequence substantially identical (e.g., at least 80%, 85%, 90%, 92%, 95%, 97%, 98%, 99% or higher identical) to SEQ ID NO: 25 or 26, or a sequence having one, two, three, four, five or more changes, e.g., amino acid substitutions, insertions, or deletions, relative to SEQ ID NO: 25 or 26, or any fragment thereof.

(exemplary nuclear localization sequence)
SEQ ID NO: 25
APKKKRKVGIHGVPAA
(exemplary nuclear localization sequence)
SEQ ID NO: 26
KRPAATKKAGQAKKKK

In some embodiments, the CRISPR/Cas9-based system may include a dCas9 molecule and a modulator of gene expression, or a nucleic acid encoding a dCas9 molecule and a modulator of gene expression. In one embodiment, the dCas9 molecule and the modulator of gene expression are linked covalently. In one embodiment, the modulator of gene expression is covalently fused to the dCas9 molecule directly. In one embodiment, the modulator of gene expression is covalently fused to the dCas9 molecule indirectly, e.g., via a non-modulator or linker, or via a second modulator. In one embodiment, the modulator of gene expression is at the N-terminus and/or C-terminus of the dCas9 molecule. In one embodiment, the dCas9 molecule and the modulator of gene expression are linked non-covalently. Exemplary sequences include but are not limited to those listed in Table 2. In some embodiments, the linker between the dCas9 and the at least one modulator of gene expression comprises an amino acid sequence corresponding to a linker listed in Table 2.

TABLE 2
Exemplary linker sequences

| Description | Sequence | SEQ ID NO: |
|---|---|---|
| linker | SSGNSNANSRGPSFSSGLVPLSLRGSH | SEQ ID NO: |
| | | 19 |
| XTEN80 | GGPSSGAPPPSGGSPAGSPTSTEEGTSESATPESGPGTST | SEQ ID NO: |
| linker | EPSEGSAPGSPAGSPTSTEEGTSTEPSEGSAPGTSTEPSE | 20 |
| XTEN16 | SGSETPGTSESATPES | SEQ ID NO: |
| linker | | 21 |

In one embodiment, the dCas9 molecule is fused to a first tag, e.g., a first peptide tag. In one embodiment, the modulator of gene expression is fused to a second tag, e.g., a second peptide tag. In one embodiment, the first and second tag, e.g., the first peptide tag and the second peptide tag, non-covalently interact with each other, thereby brining the dCas9 molecule and the modulator of gene expression into close proximity.

In one embodiment, the CRISPR/Cas9-based system includes a fusion molecule or a nucleic acid encoding a fusion molecule. In one embodiment, the fusion molecule comprises a sequence comprising a dCas9 fused to a modulator of gene expression. In one embodiment, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof.

In one embodiment, the fusion molecule is a DNMT3A-DNMT3L(3A3L)-dCas9-KRAB fusion molecule comprising from the N-terminus to the C-terminus: a DNMT3A-DNMT3L fusion peptide (3A3L), a dCas9 peptide, and a KRAB peptide domain, fused directly or indirectly (e.g., via a linker).

In one embodiment, the fusion molecule comprises the fusion molecule comprises the amino acid sequence of SEQ ID NO: 97, a sequence substantially identical (e.g., at least 80%, 85%, 90%, 91%, 92%, 93%, 94%, 95%, 96%, 97%, 98%, 99% or higher in sequence identity) to SEQ ID NO: 97, or a sequence having one, two, three, four, five or more changes, e.g., substitutions, insertions, or deletions, relative to SEQ ID NO: 97, or any fragment thereof.

DNMT3A-DNMT3L(3A3L)-dCas9-KRAB
(SEQ ID NO: 97)
MNHDQEFDPPKVYPPVPAEKRKPIRVLSLEDGIATGLLVLKDLGIQVDRYIASEVCEDSITV
GMVRHQGKIMYVGDVRSVTQKHIQEWGPFDLVIGGSPCNDLSIVNPARKGLYEGTGRLFFEF
YRLLHDARPKEGDDRPFFWLFENVVAMGVSDKRDISRFLESNPVMIDAKEVSAAHRARYFWG
NLPGMNRPLASTVNDKLELQECLEHGRIAKFSKVRTITTRSNSIKQGKDQHFPVFMNEKEDI
LWCTEMERVFGFPVHYTDVSNMSRLARQRLLGRSWSVPVIRHLFAPLKEYFACVSSGNSNAN
SRGPSFSSGLVPLSLRGSHMGPMEIYKTVSAWKRQPVRVLSLERNIDKVLKSLGFLESGSGS
GGGTLKYVEDVTNVVRRDVEKWGPFDLVYGSTQPLGSSCDRCPGWYMFQFHRILQYALPRQE
SQRPFFWIFMDNLLLTEDDQETTTRFLQTEAVTLQDVRGRDYQNAMRVWSNIPGLKSKHAPL
TPKEEEYLQAQVRSRSKLDAPKVDLLVKNCLLPLREYFKYFSQNSLPLGGPSSGAPPPSGGS
PAGSPTSTEEGTSESATPESGPGTSTEPSEGSAPGSPAGSPTSTEEGTSTEPSEGSAPGTST
EPSEPKKKRKVMDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGL
FGNLIALSLGLTPNEKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDELDN
EENEDILEDIVLTLTLFEDREMIEERLKTYAHLEDDKVMKQLKRRRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGIT
IMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVL
SAYNKHRDKPIREQAENIIHLFTLTNLGAPAAFKYFDTTIDRKRYTSTKEVLDATLIHQSIT
GLYETRIDLSQLGGDAYPYDVPDYASLGSGSPKKKRKVEDPKKKRKVDGSGSETPGTSESAT
PESRTLVTFKDVFVDFTREEWKLLDTAQQIVYRNVMLENYKNLVSLGYQLTKPDVILRLEKG
EEP

gRNA

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequence may form a duplex with a target sequence. The duplex may be a DNA duplex, an RNA duplex, or a RNA/DNA duplex. The terms “guide molecule” and “guide RNA” and “single guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

The guide molecule or guide RNA of a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In general, a CRISPR-Cas system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

As described above, the CRISPR/Cas9 system utilizes gRNA that provides the targeting of the CRISPR/Cas9-based system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

The term “target region”, “target sequence” or “protospacer” as used interchangeably herein refers to the region of the target gene to which the CRISPR/Cas9-based system targets. The CRISPR/Cas9-based system may include at least one gRNA, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the S. pyogenes Type II system uses an “NGG” sequence, where “N” can be any nucleotide.

In some embodiments, the number of gRNA administered to the cell may be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 19 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs.

In some embodiments, the number of gRNAs administered to the cell may be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

In some embodiments, the gRNA is selected to increase or decrease transcription of a target gene. In some embodiment, the gRNA targets a region upstream of the transcription start site (TSS) of a target gene (e.g. PCSK9), e.g., between 0-1000 bp upstream of the transcription start site of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the target gene. In one embodiment, the gRNA targets a region 0-300 bp upstream of the TSS of the target gene.

In some embodiments, the gRNA targets a region downstream of the transcription start site of a target gene, e.g., between 0-1000 bp downstream of the transcription start site of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp downstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the target gene. In one embodiment, the gRNA targets a region 0-300 bp downstream of the TSS of the target gene.

Proprotein Convertase Subtilisin/Kexin Type 9 (PCSK9) may also be referred to as Subtilisin/Kexin-Like Protease PC9. Human PCSK9 has a cytogenetic location of 1p32.3 and the genomic coordinates are on Chromosome 1 on the forward strand at position 55,039,548-55,064,852. BSND is the gene upstream of PCSK9 on the forward strand. Human PCSK9 has a NCBI gene ID of 255738, Ref Seq Accession No. of NM_174936.4, Ref Seq Accession No. of NP_777596.2 and Ensembl Gene ID of ENSG00000169174.

Mouse PCSK9 has a genomic location of 4, 4 C7 and has the genomic sequence of chromosome 4 at position NC_000070.07. BSND is the gene upstream of mouse PCSK9 on the forward strand. Mouse PCSK9 has a NCBI Gene ID of 100102, Ref Seq Accession No. of NM_153565.2, Ref Seq Accession No. of NP_705793.1 and Ensembl Gene ID of ENSMUSG00000044254.

Rhesus monkey (Macaca mulatta) PCSK9 has a genomic location of NC_041754.1. ENSMMUG0000005740 is the gene upstream of monkey PCSK9 on the forward strand. Monkey PCSK9 has a Ref Seq Accession No. of NM_001112660.1, Ref Seq Accession No. of NP_001106130.1 and Ensembl Gene ID of ENSMMUG00000005736.

The present disclosure provides sgRNA sequences that target a mouse PCSK9 target gene. Exemplary sgRNAs include but are not limited to those listed in Table 3. The present disclosure also provides sgRNA sequences that target human PCSK9. Exemplary sgRNAs include but are not limited to those listed in Table 4. The present disclosure also provides sgRNA sequences that target monkey PCSK9. Exemplary sgRNAS include but are not limited to those listed in Table 5.

TABLE 3
Exemplary Mouse PCSK9 sgRNA sequences

| Description | Sequence | SEQ ID NO: |
|---|---|---|
| mouse PCSK9 sgRNA1 | TGGACGCGCAGGCTGCCGGT | SEQ ID NO: 27 |
| mouse PCSK9 sgRNA2 | CCACCTTCACGTGGACGCGC | SEQ ID NO: 28 |
| mouse PCSK9 sgRNA3 | GTGGACGCGCAGGCTGCCGG | SEQ ID NO: 29 |
| mouse PCSK9 sgRNA4 | CTCTCTCTTTCTGAGGCTAG | SEQ ID NO: 30 |
| mouse PCSK9 sgRNA5 | CACGTGGACGCGCAGGCTGC | SEQ ID NO: 31 |
| mouse PCSK9 sgRNA6 | TTAAGAGGGGGGAATGTAAC | SEQ ID NO: 32 |
| mouse PCSK9 sgRNA7 | AACCTGATCCTTTAGTACCG | SEQ ID NO: 33 |
| mouse PCSK9 sgRNA8 | TCAGAGAGGATCTTCCGATG | SEQ ID NO: 34 |
| mouse PCSK9 sgRNA9 | GGATCTTCCGATGGGGCTCG | SEQ ID NO: 35 |
| mouse PCSK9 sgRNA10 | GCGTCATTTGACGCTGTCTG | SEQ ID NO: 36 |
| mouse PCSK9 sgRNA11 | TCATTTGACGCTGTCTGGGG | SEQ ID NO: 37 |
| mouse PCSK9 sgRNA12 | GATCCTTTAGTACCGGGGCC | SEQ ID NO: 38 |
| mouse PCSK9 sgRNA13 | TGCAGCCCAATTAGGATTTG | SEQ ID NO: 39 |

TABLE 4
Exemplary Human PCSK9 sgRNA sequences

| Description | Sequence | SEQ ID NO: |
|---|---|---|
| human PCSK9 sgRNA1 | GGCTCAGACCCTGAACTGAA | SEQ ID NO: 40 |
| human PCSK9 sgRNA2 | GAGGCGCTCATGGTTGCAGG | SEQ ID NO: 41 |
| human PCSK9 sgRNA3 | TCAGACCCTGAACTGAACGG | SEQ ID NO: 42 |
| human PCSK9 sgRNA4 | AGGCGCTCATGGTTGCAGGC | SEQ ID NO: 43 |
| human PCSK9 sgRNA5 | AGTTCAGGGTCTGAGCCTGG | SEQ ID NO: 44 |
| human PCSK9 sgRNA6 | CGGGCGCCGCCGTTCAGTTC | SEQ ID NO: 45 |
| human PCSK9 sgRNA7 | GGGCGCCGCCGTTCAGTTCA | SEQ ID NO: 46 |
| human PCSK9 sgRNA8 | ACTGCCTGGCTCACTCCTCC | SEQ ID NO: 47 |
| human PCSK9 sgRNA9 | TGAGCCTGGAGGAGTGAGCC | SEQ ID NO: 48 |
| human PCSK9 sgRNA10 | TTCAGTTCAGGGTCTGAGCC | SEQ ID NO: 49 |
| human PCSK9 sgRNA11 | CCAGGCAGTGAGACTGGCTC | SEQ ID NO: 50 |
| human PCSK9 sgRNA12 | TGAGCGCCTCGACGTCGCTG | SEQ ID NO: 51 |
| human PCSK9 sgRNA13 | AGGTTTCCGCAGCGACGTCG | SEQ ID NO: 52 |
| human PCSK9 sgRNA14 | GCCAGGCAGTGAGACTGGCT | SEQ ID NO: 53 |
| human PCSK9 sgRNA15 | GTCGAGGCGCTCATGGTTGC | SEQ ID NO: 54 |
| human PCSK9 sgRNA16 | CCCGAGCCAGTCTCACTGCC | SEQ ID NO: 55 |
| human PCSK9 sgRNA17 | GGCAGTGAGACTGGCTCGGG | SEQ ID NO: 56 |
| human PCSK9 sgRNA18 | CAGCGACGTCGAGGCGCTCA | SEQ ID NO: 57 |
| human PCSK9 sgRNA19 | GCAGTGAGACTGGCTCGGGC | SEQ ID NO: 58 |
| human PCSK9 sgRNA20 | GAGACTGGCTCGGGCGGGCC | SEQ ID NO: 59 |
| human PCSK9 sgRNA21 | TGAGACTGGCTCGGGCGGGC | SEQ ID NO: 60 |
| human PCSK9 sgRNA22 | GGAGACCTAGAGGCCGTGCG | SEQ ID NO: 61 |
| human PCSK9 sgRNA23 | CGGCGGCGCCTTGAGCCTTG | SEQ ID NO: 62 |
| human PCSK9 sgRNA24 | GCGCCTTGAGCCTTGCGGTG | SEQ ID NO: 63 |
| human PCSK9 sgRNA25 | GGCGCCTTGAGCCTTGCGGT | SEQ ID NO: 64 |
| human PCSK9 sgRNA26 | CCTCCCCACCGCAAGGCTCA | SEQ ID NO: 65 |
| human PCSK9 sgRNA27 | GCACAGTCCTCCCCACCGCA | SEQ ID NO: 66 |
| human PCSK9 sgRNA28 | CGGCGCCTTGAGCCTTGCGG | SEQ ID NO: 67 |
| human PCSK9 sgRNA29 | AGGCCGTGCGCGGTCCACGC | SEQ ID NO: 68 |
| human PCSK9 sgRNA30 | CTGTGCAGGAGCTGAAGTTC | SEQ ID NO: 69 |
| human PCSK9 sgRNA31 | CCTTGAGCCTTGCGGTGGGG | SEQ ID NO: 70 |
| human PCSK9 sgRNA32 | GTGGACCGCGCACGGCCTCT | SEQ ID NO: 71 |
| human PCSK9 sgRNA33 | GGCTCAAGGCGCCGCCGGCG | SEQ ID NO: 72 |
| human PCSK9 sgRNA34 | TGCGGTGGGGAGGACTGTGC | SEQ ID NO: 73 |
| human PCSK9 sgRNA35 | CGCAAGGCTCAAGGCGCCGC | SEQ ID NO: 74 |
| human PCSK9 sgRNA36 | CCGTGCGCGGTCCACGCCGG | SEQ ID NO: 75 |
| human PCSK9 sgRNA37 | CCGCCGGCGTGGACCGCGCA | SEQ ID NO: 76 |
| human PCSK9 sgRNA38 | GGAGCTGACGGTGCCCATGA | SEQ ID NO: 77 |
| human PCSK9 sgRNA39 | TCAGGAGCAGGGCGCGTGAA | SEQ ID NO: 78 |
| human PCSK9 sgRNA40 | GGGACGCGTCGTTGCAGCAG | SEQ ID NO: 79 |
| human PCSK9 sgRNA41 | GAGAGGTTGCTGTCCTGGCG | SEQ ID NO: 80 |
| human PCSK9 sgRNA42 | TGAAGGGGCGCGCGGAATCC | SEQ ID NO: 81 |
| human PCSK9 sgRNA43 | ACCTCTCCCCTGGCCCTCAT | SEQ ID NO: 82 |
| human PCSK9 sgRNA44 | GCGGCTCCCAGCTCCCAGCC | SEQ ID NO: 83 |
| human PCSK9 sgRNA45 | CAAATCCTAACTGGGCTGGA | SEQ ID NO: 84 |
| human PCSK9 sgRNA46 | TCCCGCCTCTCACCCTGCGT | SEQ ID NO: 85 |
| human PCSK9 sgRNA47 | GGAGTCTGGCATCCCACGCA | SEQ ID NO: 86 |
| human PCSK9 sgRNA48 | AAACCTGATCCTCCAGTCCG | SEQ ID NO: 87 |
| human PCSK9 sgRNA49 | GTGTGGGTGCTTGACGCCTG | SEQ ID NO: 88 |
| human PCSK9 sgRNA50 | TTAAACATTAACGGAACCCC | SEQ ID NO: 89 |
| human PCSK9 sgRNA51 | AACCTGATCCTCCAGTCCGG | SEQ ID NO: 90 |
| human PCSK9 sgRNA52 | GCCAGACTCCAAGTTCTGCC | SEQ ID NO: 91 |
| human PCSK9 sgRNA53 | ATCCCACGCAGGGTGAGAGG | SEQ ID NO: 92 |
| human PCSK9 sgRNA54 | GGCATCCCACGCAGGGTGAG | SEQ ID NO: 93 |
| human PCSK9 sgRNA55 | GCGGAAACCTTCTAGGGTGT | SEQ ID NO: 94 |
| human PCSK9 sgRNA56 | ATCGTCCGATGGGGCTCTGG | SEQ ID NO: 95 |

TABLE 5
Exemplary Monkey PCSK9 sgRNA sequences

| Description | Sequence | SEQ ID NO: |
|---|---|---|
| monkey PCSK9 sgRNA1 | CTGTGCACAGGCTTCATCAT | SEQ ID NO: 98 |
| monkey PCSK9 sgRNA2 | GCACAGTAACAACCCCTGGT | SEQ ID NO: 99 |
| monkey PCSK9 sgRNA3 | GGTCCCAAAGAGCAGCAGCA | SEQ ID NO: 100 |
| monkey PCSK9 sgRNA4 | CTAGGAGGAGGCCTTTGATG | SEQ ID NO: 101 |
| monkey PCSK9 sgRNA5 | AACAACCCCTGGTAGGTGAG | SEQ ID NO: 102 |
| monkey PCSK9 sgRNA6 | CTGGTAGGTGAGAGGCCAAG | SEQ ID NO: 103 |
| monkey PCSK9 sgRNA7 | CTGGGTGCACAGTAACAACC | SEQ ID NO: 104 |
| monkey PCSK9 sgRNA8 | TCCATTCTTTCTCTAGGAGG | SEQ ID NO: 105 |
| monkey PCSK9 sgRNA9 | TGTCCCTCTGTGCACAGGCT | SEQ ID NO: 106 |
| monkey PCSK9 sgRNA10 | GGGTGCACAGTAACAACCCC | SEQ ID NO: 107 |
| monkey PCSK9 sgRNA11 | TCCAGTTACAGGCAACAGGA | SEQ ID NO: 108 |

In one embodiment, the gRNA targets a promoter region of a target gene. In one embodiment, the gRNA targets an enhancer region of a target gene. gRNA can be divided into a target binding region, a Cas9 binding region, and a transcription termination region. The target binding region hybridizes with a target region in a target gene. Methods for designing such target binding regions are known in the art, see, e.g., Doench et al., Nat Biotechnol. (2014) 32: 1262-7; and Doench et al., Nat Biotechnol. (2016) 34: 184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

In one embodiment, the target binding region is engineered to improve stability or extend half-life, e.g., by incorporating a non-natural nucleotide or a modified nucleotide in the target binding region, by removing or modifying an RNA destabilizing sequence element, by adding an RNA stabilizing sequence element, or by increasing the stability of the Cas9/gRNA complex. In one embodiment, the target binding region is engineered to enhance its transcription. In one embodiment, the target binding region is engineered to reduce secondary structure formation. In one embodiment, the Cas9 binding region of gRNA is modified to enhance the transcription of the gRNA. In one embodiment, the Cas9 binding region of gRNA is modified to improve stability or assembly of the Cas9/gRNA complex.

### Delivery Systems

The present disclosure also provides delivery systems for introducing components of the systems and compositions herein to cells, tissues, organs, or organisms. A delivery system may comprise one or more delivery vehicles and/or cargos.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516.

### Physical Delivery

In some embodiments, the cargos may be introduced to cells by physical delivery methods. Examples of physical methods include microinjection, electroporation, and hydrodynamic delivery.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present disclosure may a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSecl, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the -actin promoter, the phosphoglycerol kinase (PGK) promoter, and the EF1a promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno-Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)) and WO 2021/183807A1, which are incorporated by reference herein in their entirety.

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin 0, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

In some embodiments, LNPs are used for delivering an mRNA and gRNAs (e.g. mRNA fusion molecule comprising DNMT3A-DNMT3L(3A-3L)-dCas9-KRAB and at least one sgRNA targeting PCSK9.

Components of LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Conway et al, Molecular Therapy, vol. 27, no. 4, pages 866-877, April 2019 and Rosin et al, Molecular Therapy, vol. 19, no. 12, pages 1286-2200, December 2011.

In some embodiments, LNPs may comprise ionizable lipids. In some embodiments, ionizable lipids include but are not limited to pH-responsive ionizable lipids, thermal-responsive ionizable lipids and light-responsive ionizable lipids. In some embodiments, ionizable lipids include cationic lipids and anionic lipids that are ionized under the certain conditions, such as, but not limited to pH, temperature or light. In some embodiments, the molar ratio of ionizable lipids of the LNP is 20% to about 70% (e.g., about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 30% to about 70%, about 30% to about 65%, about 30% to about 60%, about 30% to about 55%, about 30% to about 50%, about 30% to about 45%, about 30% to about 40%, about 30% to about 35%, about 40% to about 70%, about 40% to about 65%, about 40% to about 60%, about 40% to about 55%, about 40% to about 50%, about 40% to about 45%, about 50% to about 70%, about 50% to about 65%, about 50% to about 60%, about 50% to about 55%, about 60% to about 70%, or about 60% to about 65%). In some embodiments, the molar ratio of ionizable lipids of the LNP is about 45% to about 50%.

In some embodiments, LNPs may comprise PEGylated lipids. In some embodiments, the molar ratio of PEGylated lipids of the LNP is 0% to about 30% (e.g., about 0% to about 30%, about 0% to about 25%, about 0% to about 20%, about 0% to about 15%, about 0% to about 10%, about 10% to about 30%, about 10% to about 25%, about 10% to about 20%, about 10% to about 15%, about 20% to about 30%, or about 20% to about 25%). In some embodiments, the molar ratio of PEGlyated lipids of the LNP is about 1%.

In some embodiments, LNPs may comprise supporting lipids. In some embodiments, the molar ratio of supporting lipids of the LNP is 5% to about 50% (e.g. about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 50%, about 10% to about 45%, about 10% to about 40%, about 10% to about 35%, about 10% to about 30%, about 10% to about 25%, about 10% to about 20%, about 10% to about 15%, about 20% to about 50%, about 20% to about 45%, about 20% to about 40%, about 20% to about 35%, about 20% to about 30%, about 20% to about 25%, about 30% to about 50%, about 30% to about 45%, about 30% to about 40%, about 30% to about 35%, about 40% to about 50%, about 40% to about 45%, about 30% to about 50%, about 30% to about 45%, about 30% to about 40%, about 30% to about 35%, about 40% to about 50%, or about 40% to about 45%). In some embodiments, the molar ratio of supporting lipids of the LNP is about 9%.

In some embodiments, LNPs may comprise cholesterol. In some embodiments, the molar ratio of cholesterol of the LNP is 10% to about 50% (e.g., about 10% to about 50%, about 10% to about 45%, about 10% to about 40%, about 10% to about 35%, about 10% to about 30%, about 10% to about 25%, about 10% to about 20%, about 10% to about 15%, about 20% to about 50%, about 20% to about 45%, about 20% to about 40%, about 20% to about 35%, about 20% to about 30%, about 20% to about 25%, about 30% to about 50%, about 30% to about 45%, about 30% to about 40%, about 30% to about 35%, about 40% to about 50% or about 40% to about 45%). In some embodiments, the molar ratio of cholesterol of the LNP is about 40% to about 45%.

In some embodiments, LNPs may comprise a mixture of ionizable lipids (20%-70%, molar ratio), PEGylated lipids (0%-30%, molar ratio), supporting lipids (5%-50%, molar ratio), and cholesterol (10%-50%, molar ratio). In some embodiments, the LNPs may comprise a mixture of ionizable lipids (45-50%, molar ratio), PEGylated lipids (1% molar ratio), supporting lipids (9%, molar ratio), and cholesterol (40-50%, molar ratio).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, Cl2-200 and colipids disteroylphosphatidyl choline, cholesterol, and PEG-DMG.

Lipoplexes and/or Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²+ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus I (HIV-I). Examples of CPPs include to Penetratin, Tat (48-60), Transportan, and (R-AhX-R4) (Ahx refers to aminohexanoyl). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

### DNA Nanoclews

In some embodiments, the delivery vehicles comprise DNA nanoclews. A DNA nanoclew refers to a sphere-like structure of DNA (e.g., with a shape of a ball of yam). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Casl3a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci US A 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Ace Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates Kand Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Methods of Use

The compositions and systems herein may be used for a variety of applications, including modifying non-animal organisms such as plants and fungi, and modifying animals, treating and diagnosing diseases in plants, animals, and humans. In general, the compositions and systems may be introduced to cells, tissues, organs, or organisms, where they modify the expression and/or activity of one or more genes (e.g. PCSK9).

In some embodiments, the expression of a PCSK9 gene product is reduced in a cell introduced with the composition and systems described herein. In some embodiments, the reduction of PCSK9 gene product expression is transient. In some embodiments, the reduction of PCSK9 gene product expression is stable. In some embodiments, the reduction of PCSK9 gene product expression is heritable.

In some embodiments, the plurality of cells modified by the composition and systems herein comprises at least 10%, at least 20%, at least 30% at least 40%, at least 50%, at least 60%, at least 70%, at least 80% or at least 90% reduced expression of the PCSK9 gene product relative to a cell that has not been introduced with the composition and system described herein.

In some embodiments, cells expanded or derived from the plurality of cells modified by the composition and systems described herein also comprise at least 10%, at least 20%, at least 30% at least 40%, at least 50%, at least 60%, at least 70%, at least 80% or at least 90% reduced expression of the PCSK9 gene product relative to a cell expanded or derived from a cell that has not been introduced with the composition and system described herein.

### Cells and Organisms

The present disclosure provides cells, tissues, organisms comprising the engineered Cas protein, the CRISPR-Cas systems, the polynucleotides encoding one or more components of the CRISPR-Cas systems, and/or vectors comprising the polynucleotides. The disclosure also provides for the nucleotide sequence encoding the effector protein being codon optimized for expression in a eukaryote or eukaryotic cell in any of the herein described methods or compositions. In an embodiment of the disclosure, the codon optimized effector protein is any Cas protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome. In certain embodiments, the gene product may be altered in comparison with the gene product from a cell that does not have altered expression or edited genome.

### Exemplary Therapies

The present disclosure provides a use of the CRISPR-Cas system for treatment in of a variety of diseases and disorders. In some embodiments, the disclosure described herein relates to a method for therapy in which cells are edited ex vivo by CRISPR or the base editor to modulate at least one gene, with subsequent administration of the edited cells to a patient in need thereof. In some embodiments, the editing involves knocking in, knocking out or knocking down expression of at least one target gene in a cell. In particular embodiments, the editing inserts an exogenous, gene, minigene or sequence, which may comprise one or more exons and in trans or natural or synthetic in trans into the locus of a target gene, a hot-spot locus, a safe harbor locus of the gene genomic locations where new genes or genetic elements can be introduced without disrupting the expression or regulation of adjacent genes, or correction by insertions or deletions one or more mutations in DNA sequences that encode regulatory elements of a target gene. In some embodiment, the editing comprise introducing one or more point mutations in a nucleic acid (e.g., a genomic DNA) in a target cell.

In some embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

In some embodiments, the disease is associated with high cholesterol, and regulation of cholesterol (e.g. LDL) is provided. In some embodiments, regulation is affected by modification in the target gene PCSK9. PCSK9 has been associated with diseases and disorder such as, but not limited to, Abetalipoproteinemia, Adenoma, Arteriosclerosis, Atherosclerosis, Cardiovascular Diseases, Cholelithiasis, Coronary Arteriosclerosis, Coronary heart disease, Non-Insulin-Dependent Diabetes Mellitus, Hypercholesterolemia, Familial Hypercholesterolemia, Hyperinsulinism, Hyperlipidemia, Familial Combined Hyperlipidemia, Hypobetalipoproteinemias, Chronic Kidney Failure, Liver diseases, Liver neoplasms, melanoma, Myocardial Infarction, Narcolepsy, Neoplasm Metastasis, Nephroblastoma, Obesity, Peritonitis, Pseudoxanthoma Elasticum, Cerebrovascular accident, Vascular Diseases, Xanthomatosis, Peripheral Vascular Diseases, Myocardial Ischemia, Dyslipidemias, Impaired glucose tolerance, Xanthoma, Polygenic hypercholesterolemia, Secondary malignant neoplasm of liver, Dementia, Overweight, Hepatitis C, Chronic, Carotid Atherosclerosis, Hyperlipoproteinemia Type Ha, Intracranial Atherosclerosis, Ischemic stroke, Acute Coronary Syndrome, Aortic calcification, Cardiovascular morbidity, Hyperlipoproteinemia Type lib, Peripheral Arterial Diseases, Familial Hyperaldosteronism Type II, Familial hypobetalipoproteinemia, Autosomal Recessive Hypercholesterolemia, Autosomal Dominant Hypercholesterolemia 3, Coronary Artery Disease, Liver carcinoma, Ischemic Cerebrovascular Accident, and Arteriosclerotic cardiovascular disease NOS. Epigenetic modification of the PCSK9 gene using any of the methods described herein may be used to treat, prevent and/or mitigate the symptoms of the diseases and disorders described herein.

Dyslipidemias is a genetic disease characterized by elevated level of lipids in the blood that contributes to the development of clogged arteries (atherosclerosis). These lipids include plasma cholesterol, triglycerides high-density lipoprotein or low-density lipoproteins. Dyslipidemia increases the risk of heart attacks, stroke, or other circulatory concerns. Current management includes lifestyle changes such as exercise and dietary modifications as well as use of lipid-lowering drugs such as statins. Non-statin lipid-lowering drugs include bile acid sequestrants, cholesterol absorption inhibitors, drugs for homozygous familial hypercholesteremia, fibrates, nicotinic acid, omega-3 fatty acids and/or combination products. Treatment options usually depend on the specific lipid abnormality, although different lipid abnormalities often coexist. Treatment of children is more challenging as dietary changes may be difficult to implement and lipid-lowering therapies have not been proven effective. Epigenetic modification of the PCSK9 gene using any of the methods described herein may be used to treat, prevent and/or mitigate the symptoms of dyslipidemias (e.g. LDL dysregulation).

The activity of PCSK9 is largely confined primarily to the liver and PCSK9 is associated with Dyslipidemias, PCSK9-related familial hypercholesterolemia, hypercholesterolemia (familial), gastric papillary adenocarcinoma, homozygous familial hypercholesterolemia, and nasopharyngitis, PCSK9-related familial hypercholesterolemia is an inherited disease (autosomal dominant) where the body develops dangerously blood cholesterol levels due to the lack of a receptor for the low-density lipoprotein cholesterol. PCSK9-related familial hypercholesterolemia affects between 1 in 500 heterozygotic and 1 in 1,000,000 homozygotic people worldwide and is more common in Afrikaner, French Canadians, Lebanese Christians, and Finns populations. Common symptoms of PCSK9-related familial hypercholesterolemia include elevated circulating cholesterol contained in either low-density lipoproteins alone or also in very-low-density lipoproteins. Current treatments of PCSK9-related familial hypercholesterolemia include administration of statins to inhibit hydroxymethylglutaryl CoA reductase (HMG-CoA-reductase) in the liver. Another option for treating PCSK9-related familial hypercholesterolemia is ezetimibe to inhibit cholesterol absorption in the gut.

In some embodiments, the epigenetic modification of the PCSK9 gene of any of the methods described herein can be targeted to the liver, the primary location of activity of PCSK9.

### EXAMPLES

### Example 1: Fusion Molecule Plasmid Construction and Knock Down Efficiency

Two plasmids were constructed to form the “EPICAS” system (used interchangeably with “CRISPRoff” system) (FIG. 1A). The “fusion molecule” or “catalytic protein” plasmid encodes dCas9, DNMT3A, DNMT3L and KRAB peptides. A fused DNMT3A and DNMT3L (3A3L) peptide is at the N-terminal of dCas9, and KRAB is at the C-terminal of dCas9. Thus, the fusion molecule has a 3A3L-dCas9-KRAB, from the N-terminal to the C-terminal end. The “sgRNA” plasmid encodes a sgRNA sequence that targets the PCSK9 gene. Multiple sgRNAs were designed to target the region within the 250 bp upstream and downstream of the transcription start site (TSS) of the mouse Pcsk9 gene.

Individual sgRNA plasmids were co-transfected with the catalytic protein plasmid into the mouse AML12 cell lines. After 72 hours, the top 10% GFP+ and mCherry+ cells were sorted by FACS. RT-QPCR experiments were performed to evaluate the mRNA expression level of Pcsk9. 12 out of 13 sgRNAs that were tested showed significantly down-regulated expression of Pcsk9 in AML12 cells. Cells transfected with sgRNA9 showed efficient knock-down of up to about 82% (FIG. 1B).

Next, combinations of sgRNA9 with other individual sgRNAs were tested to determine if the combination of more than one sgRNA could further reduce the gene expression level of Pcsk9 in AML12 cells (FIG. 1C). Amongst the combinations tested, sgRNA7 and sgRNA9 together showed the highest suppression levels. Individual combinations of sgRNAs were also tested to determine if the combination of more than one sgRNA could reduce the gene expression level of Pcsk9 in Ai9 primary hepatocyte cells (FIG. 1D). All the combinations significantly knocked down the expression level of Pcsk9. The lowest reduction was observed in cells that were co-transfected with sgRNA7, sgRNA8 and sgRNA9. Pcsk9 silencing persisted for at least over two weeks in primary hepatocytes, and the combination of sgRNA7 and sgRNA9 together showed the highest efficiency (up to 81%) suppression of Pcsk9 gene expression. Together, this shows that the EPICAS system can be used to induce efficient and persistent silencing of the Pcsk9 gene in mouse liver cells.

### Example 2: In Vitro Transcription of mRNA Encoding Fusion Molecule

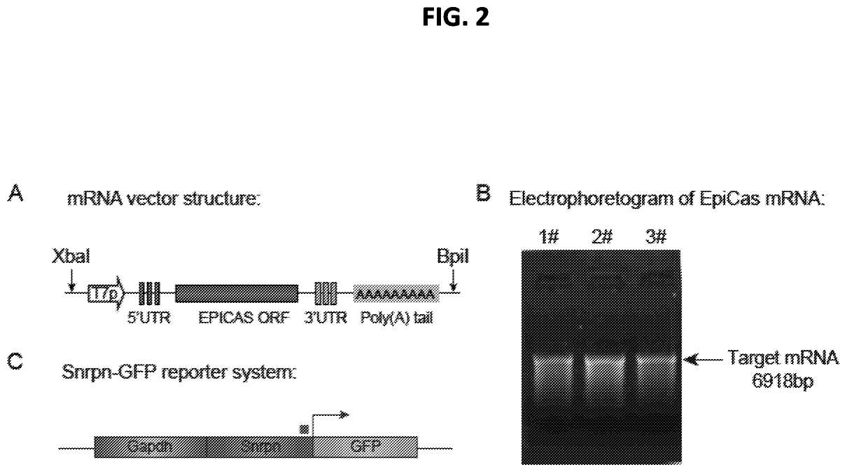

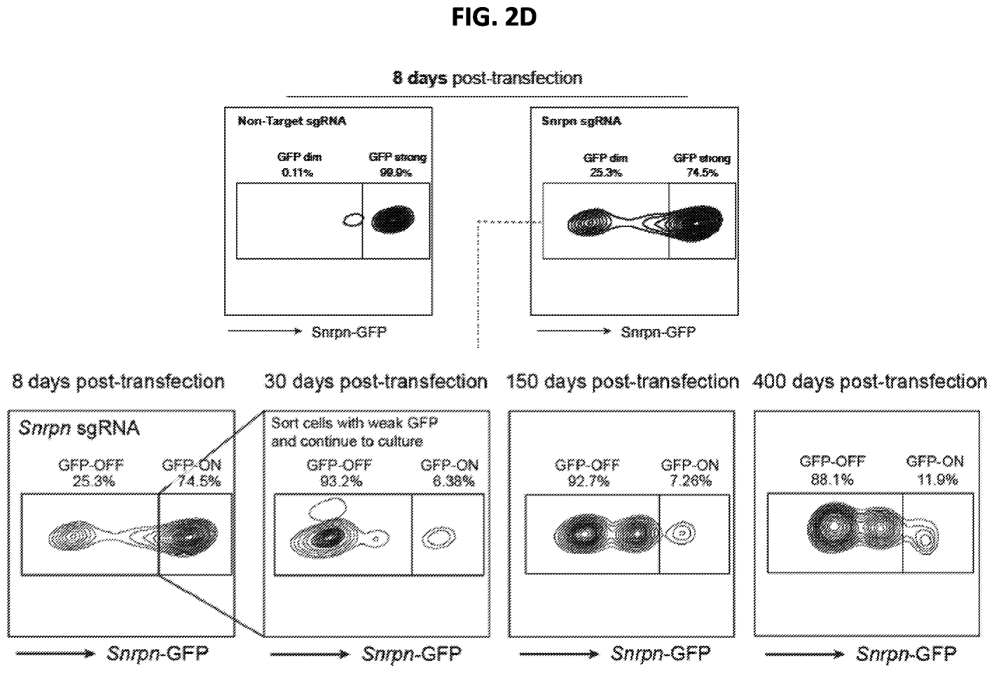

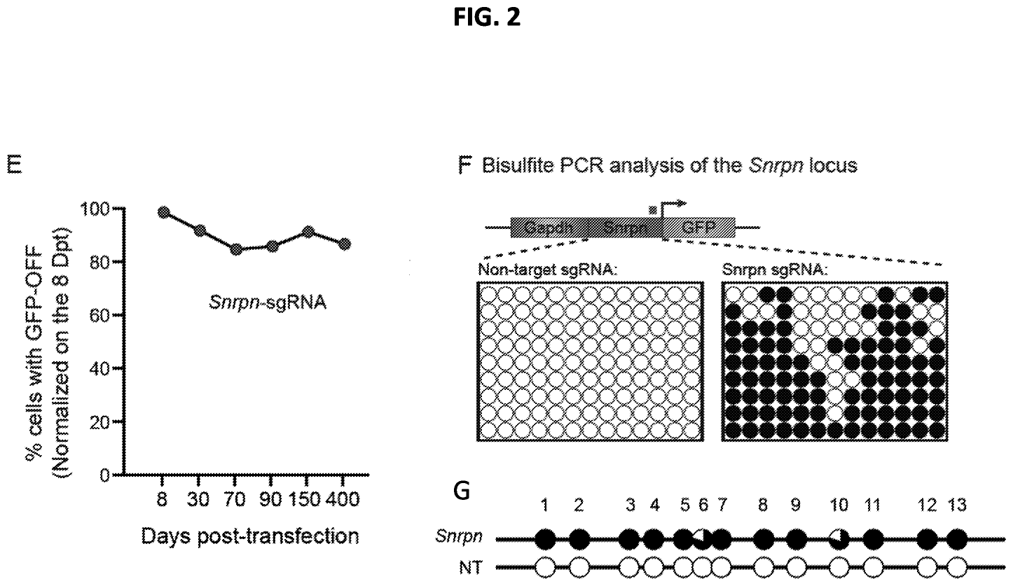

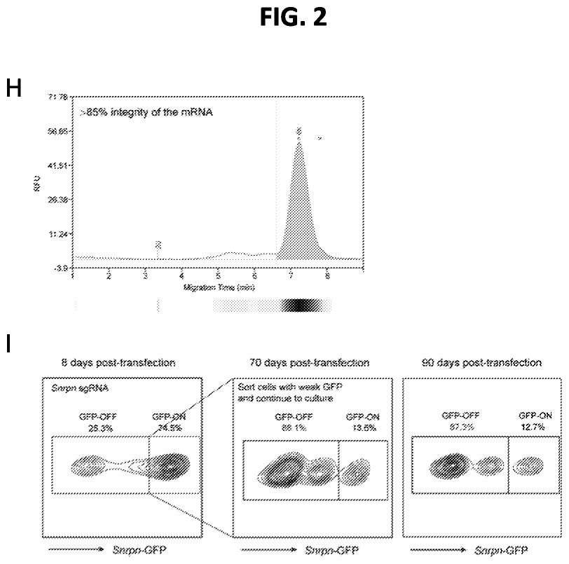

In vitro transcription and purification was used to produce mRNA corresponding to the fusion molecule or catalytic protein of the EPICAS system. First, a plasmid containing all of the fusion molecule elements, including a cassette of 5′UTR-DNMT3A-DNMT3L-dCas9-KRAB-3′UTR-polyA was constructed. The plasmid sequence was linearized by XbaI and BpiI restriction enzyme digestion (FIG. 2A). An in vitro transcription reaction containing linearized DNA template, T7 RNA polymerase, NTPs and cap analogue was performed to produce mRNA containing N1-methylpseudouridine. After digestion of the DNA template with DNase I, the mRNA product underwent purification and buffer exchange, and the purity of the final mRNA product was assessed with capillary gel electrophoresis (FIG. 2B). The 100-mer sgRNAs were chemically synthesized with minimal end-modifications under solid phase synthesis conditions by a commercial supplier. To test the function of in vitro transcribed mRNAs, a Snrpn-GFP reporter system was constructed in HEK293T cells (FIG. 2C). The reporter system controls the expression of GFP using a synthetic methylation-sensing promoter (conserved sequence elements from the promoter of an imprinted gene, Snrpn). Insertion of this reporter construct into a genomic locus showed the methylation state of the adjacent sequences. The in vitro transcribed mRNAs described above were co-transfected with sgRNA targeting the Snrpn gene into reporter cells. 8 days post-transfection, 25.3% of cells in the reporter cells were GFP-negative, which was significantly higher than that of the control group transfected with non-targeting sgRNA (FIG. 2D). The GFP-negative cells were sorted by FACS and cultured for 30 days. At 30 days post-transfection, 93.2% of cells in the reporter system were GFP-negative, whereas nearly no GFP-negative cells were found in the control group (FIGS. 2D,2E). At 70 days and 90 days post-transfection, 86.1% and 87.3% of cells in the reporter system were GFP-negative, respectively (FIG. 2I). At 150 days and 400 days post transfection (i.e. up to ˜400 cell divisions), 92.7% and 88.1% of cells in the reporter system were GFP-negative, respectively. This suggests the durability of epigenomic editing using the CRISPRoff system (FIG. 2D). Furthermore, the DNA methylation level on the Snrpn locus was analyzed by bisulfite PCR assay. The methylation level of the reporter cells (GFP-OFF group) was significantly higher than that of the control cells (GFP-ON group) (FIG. 2F). This result was accompanied by the observation of high CpG methylation on the Snrpn locus (FIGS. 2F, 2G, 2I). Together, these results show that transient expression of the EPICAS system and mRNA scan silence the expression of targeted genes for a long duration of time.

### Example 3: Lipid Nanoparticle Encapsulation of mRNA Encoding Fusion Molecules and sgRNAs

### LNP Formulation and Characterization

Standard methods known in the art were used to formulate LNPs for delivery of fusion molecule mRNA and sgRNA to human hepatocytes. For mouse studies, LNPs were formulated as previously described with some modifications (1). In short, an ethanolic solution of 1,2-distearoyl-sn-glycero-3-phosphocholine, cholesterol, a PEG lipid and an ionizable cationic lipid was rapidly mixed with an aqueous solution (pH 4) containing mRNA and sgRNA (1:1 weight ratio) using an (in-line) mixer at a flow ratio of 1:3 (ethanol: aqueous phase). A N:P ratio of 4-6 between ionizable lipids and the nucleic acids was maintained throughout the study.

The resulting LNP formulation was dialyzed overnight against 1×PBS, 0.2-μm sterile filtered and stored at 4° C. until use. The particle sizes were with the range of 70-90 nm (Z-Ave, hydrodynamic diameter), with a polydispersity index of <0.2 as determined by dynamic light scattering (Malvern NanoZS Zetasizer). Encapsulation efficiency of RNA in the LNP was measured by the Quant-iT Ribogreen Assay (Life Technologies).

### Cryo-TEM Sample Preparation and Imaging

LNP sample (3-5 μl) was dispensed on a plasma cleaned grid (Quantifoil, R1.2/1.3 300 or 400 Cu mesh) in the FEI Vitrobot chamber at 95% relative humidity and allowed to rest for 30-60 s. Then, the grid was blotted for 3 s with filter paper and plunged into liquid ethane cooled by liquid nitrogen. Cryo-EM imaging was conducted on FEI Talos F200C, operated at 200 kV accelerating voltage.

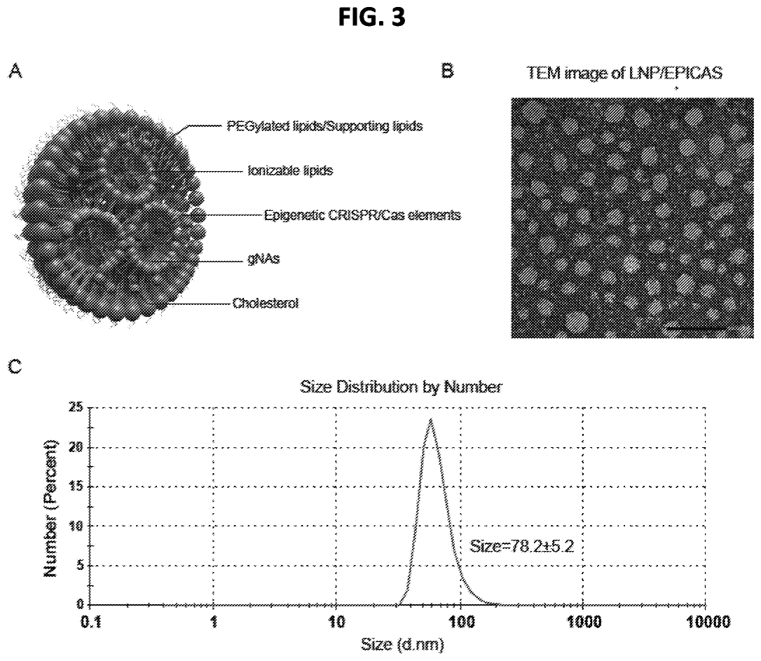

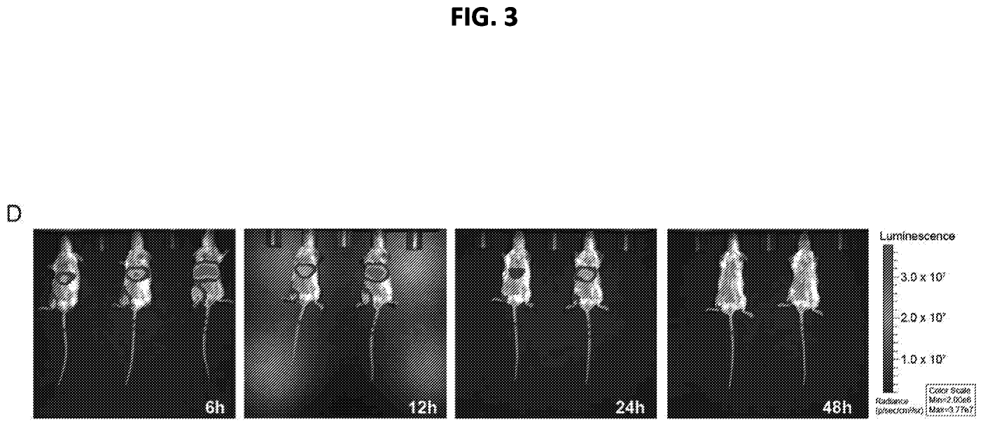

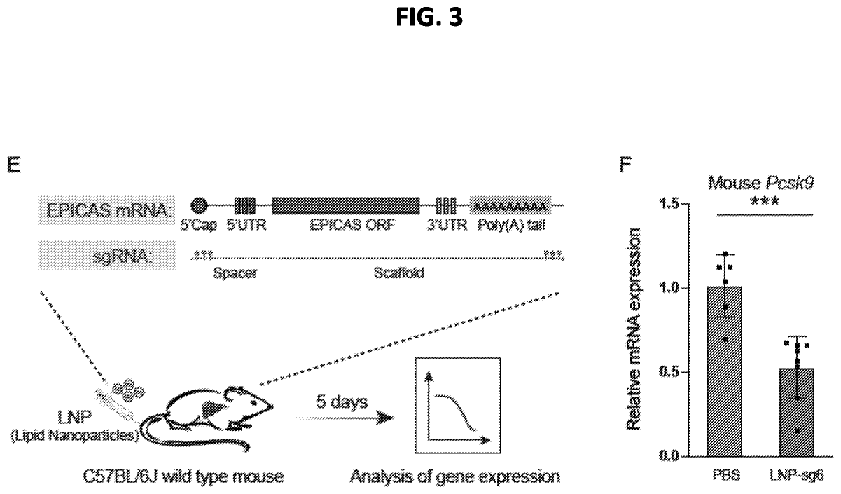

LNPs contained fusion molecule mRNA and sgRNA targeting Pcsk9 gene at a 1:1 ratio by weight (FIG. 3A). A mixture of ionizable lipids (20%-70%, molar ratio), PEGylated lipids (0%-30%, molar ratio), supporting lipids (5%-50%, molar ratio), and cholesterol (10%-50%, molar ratio), lipid nanoparticles (LNPs) were formulated using well-designed impinging stream reactors or microfluidic devices. By varying the proportion of ionizable lipids, the release kinetics of sgRNA and mRNA can be modified. With higher proportion of the ionizable lipids (molar ratio above 55%), sgRNA was released much faster than mRNA. Transmission electron microscope (TEM) images showed that the LNPs were spherical and nano-sized particles (FIG. 3B). LNPs had uniform-sizes (78.2±5.2 nm, PDI<0.10) using dynamic light scattering (NanoSZ, Malvern) (FIG. 3C).

### Example 4: PCSK9 Gene Silencing Using LNP Delivery of mRNA Encoding Fusion Molecules and sgRNAs in Mice

Next, the use of the EPICAS system (also termed “CRISPRoff” system) for silencing Pcsk9 expression in vivo was tested. The LNPs were administered to the C57CB/6J mice via injection into the lateral tail vein (FIG. 3E). Five days after injection, the mice were euthanized, and liver samples were obtained and processed for mRNA purification. RT-QPCR experiments were performed to evaluate the knock-down efficiency of Pcsk9 gene in mice. Expression levels of Pcsk9 in LNP injected mice were significantly lower than the control group (FIG. 3F), indicating the efficacy of EPICAS system in silencing Pcsk9 gene expression in vivo.

To test whether the LNPs could deliver the mRNAs successfully to mouse liver cells in vivo, LNPs containing luciferase mRNAs were produced and injected into wild-type mice by intramuscular injection.

In Vivo Luc mRNA Delivery

For detection of in vivo distribution of Luc mRNA-LNPs, female Balb/c mice aged 6-8 weeks (n=5) were in vivo injected with 5 μg of Luc mRNA. At the stated detection timepoints, mice were injected with 0.2 ml of D-luciferin (15 mg/ml in DPBS) and imaged using an IVIS Lumina system (Perkin Elmer).

For in vitro imaging, female BALB/c mice of 6-8 weeks old (n=2) were injected with 5 μg of Luc mRNA. Twelve hours later, animals were injected intraperitoneally (i.p.) with 0.2 mL D-luciferin (15 mg/ml in DPBS) followed by reaction for 5 minutes. Tissues including heart, liver, spleen, lung, and kidney were collected immediately, and the fluorescence signals of each tissue were monitored by IVIS Lumina system.

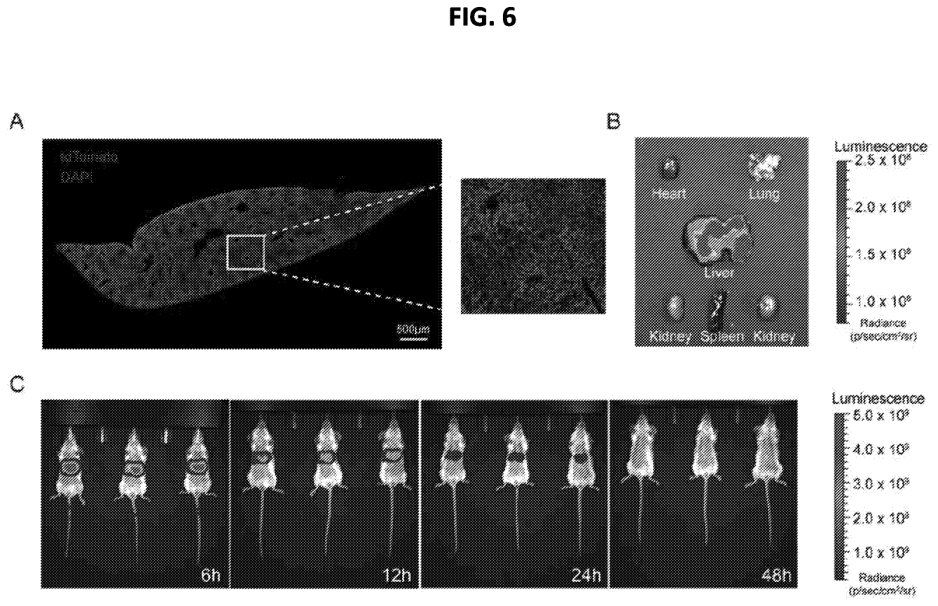

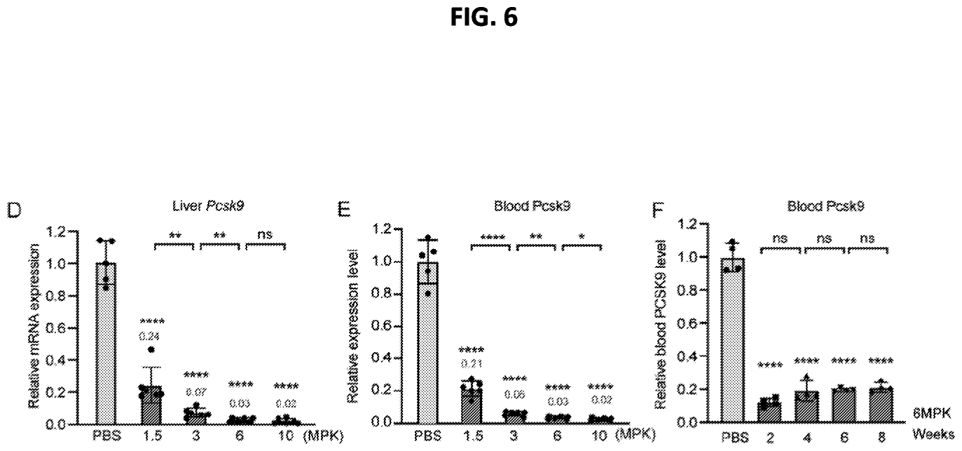

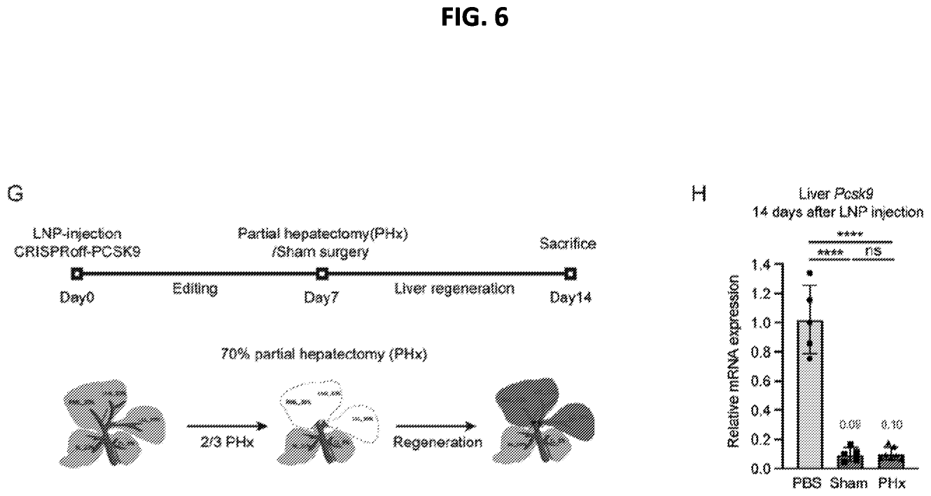

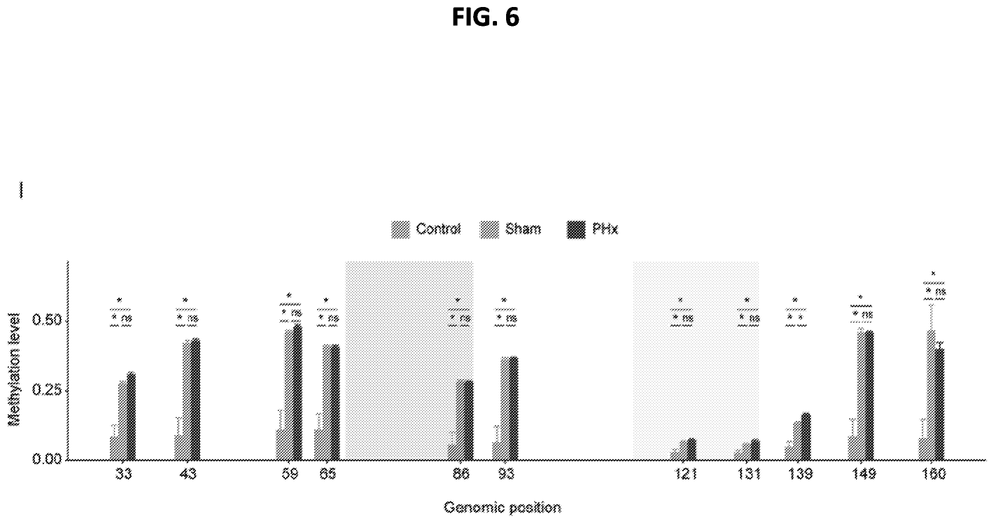

By in vivo imaging of the fluorescence, LNPs were shown to deliver the luciferase mRNAs into mouse liver cells with high efficiency (FIG. 3D). It was shown that LNPs could also deliver the relatively large sizes of mRNA via tail vein injection into the mouse liver with high efficiency, as shown by almost all of the liver cells exhibiting tdTomato fluorescence (FIG. 6A). Furthermore, LNP-delivered luciferase mRNAs accumulated in the mouse liver 6 hr after injection (FIG. 6B), gradually decreased in the mouse liver at 12 hr and 24 hr after injection, and absent in the liver 48 hr after injection (FIG. 6C).

### LNP Treatment of Mice

The mouse studies were approved by Beijing Vital River Laboratory Animal Technology Co., Ltd. And SLAC Laboratory and used for experiments. Ai9 (C57Bl/6J genetic background) and C57Bl/6J wildtype mice were maintained in specific pathogen-free husbandry and on a 12 h light/12 h dark cycle. The use and care of animals complied with the guideline of the Biomedical Research Ethics Committee of Shanghai Institutes for Biological Science, Chinese Academy of Sciences. Male C57BL/6J mice were used for experiments at 8-10 weeks of age, with random assignment of mice to various experimental groups, and with collection and analysis of data performed in a blinded fashion. LNPs were administered to the mice in 200 μl PBS via injection into the lateral tail vein. Mice were sacrificed at designated time points, and liver samples and serum were obtained on necropsy for RNA extraction or serum biochemistry analysis.

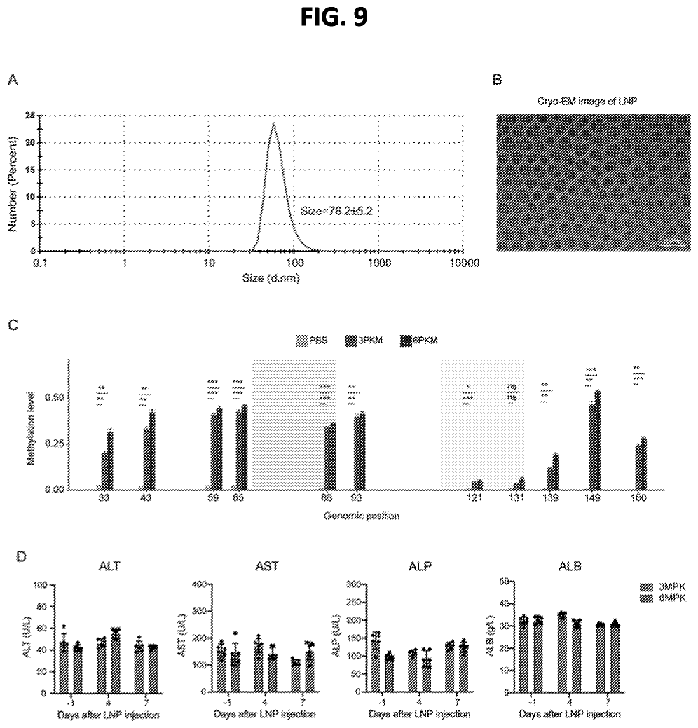

For CRISPRoff delivery in C57CB/6J adult wild-type mice, CRISPRoff mRNA and sgRNAs (sgRNA 7 (SEQ ID NO: 33) and sgRNA 9 (SEQ ID NO: 35)) at a 1:1 ratio by weight were delivered by the intravenous injection of LNPs (FIG. 9A, 9B). Liver tissues were collected for qPCR analysis at 7 days after injection. The expression of Pcsk9 was markedly reduced, as compared with that of the control mice injected with PBS (FIG. 6D). Further studies showed that doses of CRISPRoff-containing LNPs at 1.5, 3.0, 6.0, and 10 mg/kg led to 76%, 93%, 97%, and 98% suppression of the Pcsk9 expression, with near plateau effects at 3.0 mg/kg (FIG. 6D). The Pcsk9 protein level in the blood was also significantly reduced in CRISPRoff-treated mice, with dose-dependence similar to that for Pcsk9 expression in the liver tissue (FIG. 6E). A high level of DNA methylation was observed at the promoter of Pcsk9 gene at 3.0 and 6.0 mg/kg without abnormality in blood chemistry (AST, ALT, ALP and ALB) (FIG. 9C, 9D).

Stable PCSK9 Methylation and Reduced Expression after Liver Resection and Regeneration

### Partial Hepatectomy (PHx) Induced Liver Regeneration Mice Model

Mice partial hepatectomy (PHx) was performed as described before (2). Briefly, we used general anesthesia, a small upper midline incision, silk suture to tie off the lobes to be removed, warming pads and lights, as well as subcutaneous saline injection to ensure minimal morbidity.

### High-Fat Diet Induced Hypercholesterolemic Murine Model

High-fat diet mice were obtained from The GemPharmatech Co., Ltd. (Nanjing, China). They were housed in specific pathogen-free husbandry and on a 12 h light/12 h dark cycle, and fed with high-fat diet, 60 kcal % saturated (lard) fat diet (HFD) obtained from Research Diets, Inc. (New Brunswick, NJ) for 24 weeks. Mice with blood LDL-c levels greater than 25 mg/dL were selected for the experiment.

Next, the durability of CRISPRoff-induced Pcsk9 reduction at the dose of 6 mg/kg was assessed. In the blood of CRISPRoff-treated mice, the protein expression levels of Pcsk9 exhibited 88%, 81%, 82%, and 77% reduction at 2, 4, 6, and 8 weeks after LNP injection, respectively, indicating persistent silencing of the targeted gene by CRISPRoff in vivo (FIG. 6F). To further demonstrate the heritability of CRISPRoff-mediated epigenome editing, a hepatectomy experiment was conducted to measure the gene silencing effect after liver regeneration. Specifically, mice were administrated with CRISPRoff-containing LNPs at day 0, and a 70% partial hepatectomy (PHx) (14) or a sham surgery was performed at day 7 (FIG. 6G). The liver tissue samples after the PHx experiments were collected at day 14 when liver regeneration was nearly complete (FIG. 6G). The expression of Pcsk9 mRNAs in the liver after PHx showed similar levels of reduction with that in the group with sham surgery (93+/−11% and 92%+/−9%, P<0.0001, t-test) (FIG. 6H). In addition, the CpG methylation at the promoter of Pcsk9 was maintained at high level after the PHx (FIG. 6I). Since a large amount of liver cells were regenerated during the post-PHx period via cell division, these results indicate the CRISPRoff-mediated epigenome editing is heritable during cell division in vivo, which is advantageous for therapeutic design.

Persistent Reduction of Blood LDL in Mice Fed with a High Fat Diet

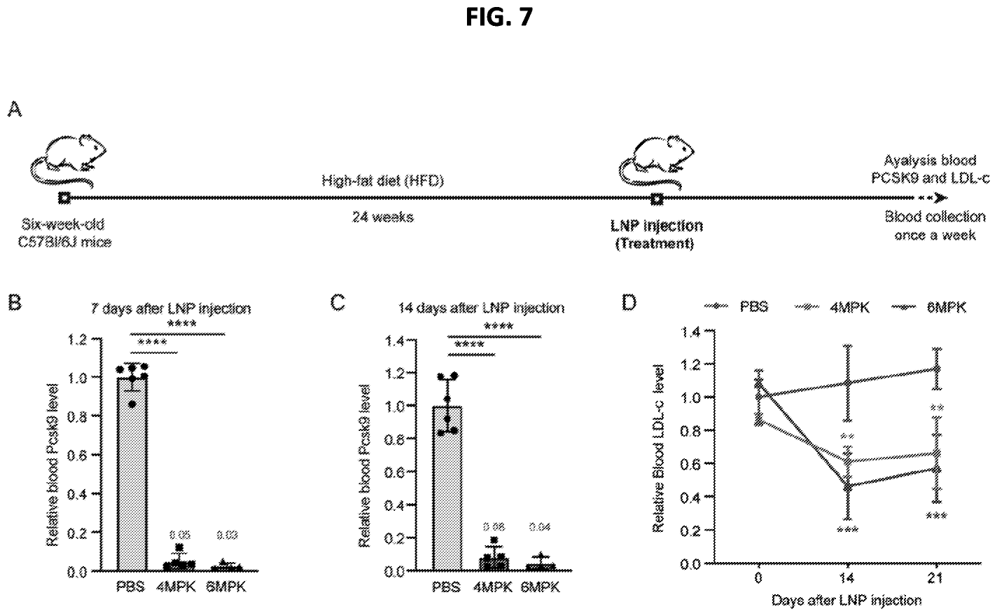

It has been shown that the serum LDL-C levels increase with high-fat diet (HFD) (15). The effects of epigenome editing on lowering Pcsk9 levels in mice fed with a HFD was evaluated. Specifically, the six-week-old male C57BI/6J mice were fed with an HFD for 6 months, and then were treated with LNPs encapsulating CRISPRoff mRNA together with sgRNAs targeting Pcsk9 or PBS via tail vein injection (FIG. 7A). At 7 and 14 days after injection, Pcsk9 levels in the blood were markedly reduced at doses of 4 mg/kg and 6 mg/kg, as compared with the PBS group (FIG. 7B, 7C). In addition, the serum LDL-C levels were lowered by about 44% and about 58% at 4 mg/kg and 6 mg/kg at 14 days after injection, and about 43% and about 51% at 21 days after injection, respectively (FIG. 7D). These results show that lowering Pcsk9 by epigenome editing could reduce the serum LDL-C levels in mice on HFD with high efficiency and persistence, which is advantageous in therapeutic design.

Precise Silencing of PCSK9 by EPICAS System without Off-Target Effects

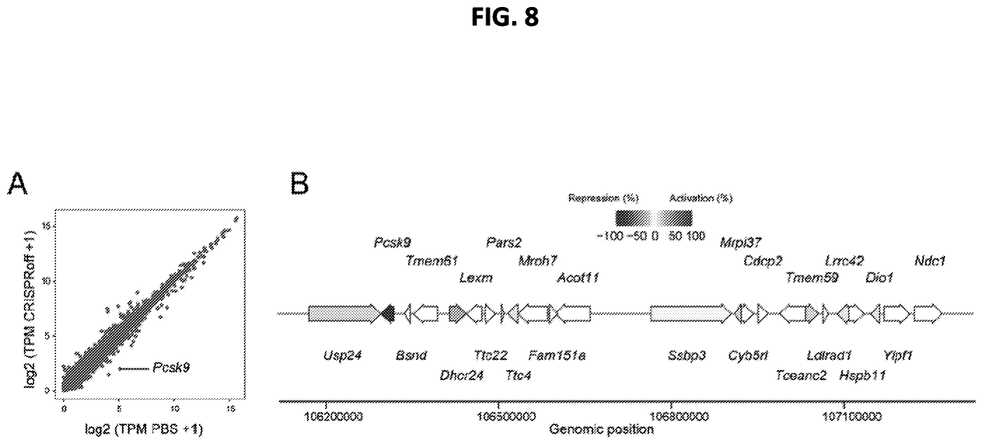

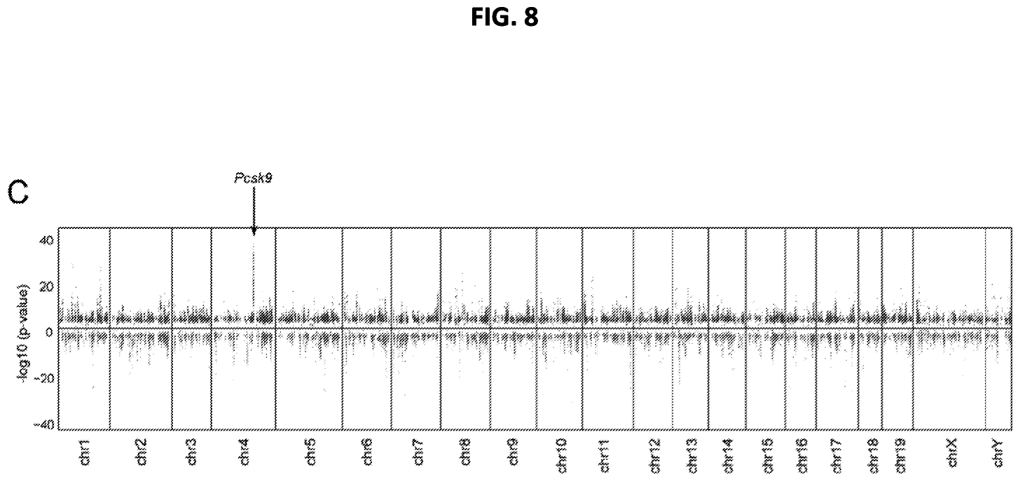

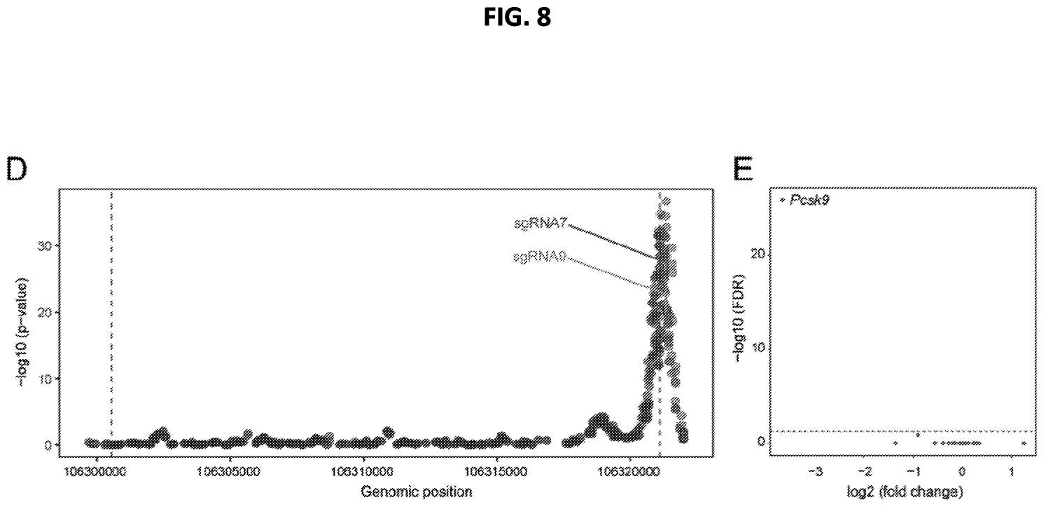

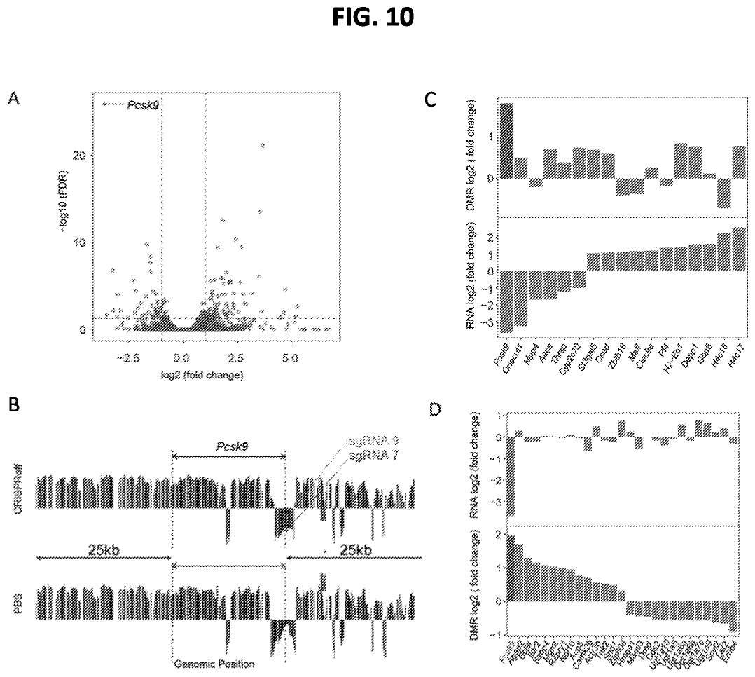

To investigate the potential off-target effects of epigenome editing, the specificity of CRISPRoff editing at both transcriptomic and genomic levels was evaluated. RNA-seq was performed on mouse liver tissues at 7 days after LNP delivery of CRISPRoff mRNA and Pcsk9-targeting sgRNAs. Transcriptome-wide gene expression levels in CRISPRoff-treated mice showed no significant difference with those found in PBS-treated mice, except that of the targeted gene Pcsk9, which was silenced (FIG. 8A and FIG. 10A). The expression levels of neighboring genes within a 1-Mb window from Pcsk9 also exhibited no significant difference between two groups of mice (FIG. 8B). Several non-target transcripts with more than 2 folds changes (FDR<0.05) in the CRISPRoff-treated group were observed (FIG. 10B), but, the DNA methylation levels on these non-target transcripts showed no significant difference between the CRISPRoff- and PBS-treated mice (FIG. 10B). Furthermore, high-throughput whole-genome bisulfite sequencing (WGBS) was performed to examine the off-target methylation of CpGs on liver tissues. Methylation levels were not significantly different between the two groups of mice at all CpG sites except for Pcsk9, which showed a dominant gain in DNA methylation at the Pcsk9 promoter in the CRISPRoff-treated mice (FIG. 8C). Detailed examination revealed that the DNA methylation was only upregulated at the promoter regions targeted by the sgRNAs, with no spreading to the Pcsk9 gene body or neighboring genes (FIG. 8D and FIG. 10C). Inspection of genes at or near the non-target differentially methylated regions revealed no transcriptional differences (FIG. 10D), suggesting that the non-specific methylation difference has little effect on gene expression. We also compared the methylation and gene expression levels of potential sgRNA-dependent off-target sites (having high sequence similarity with the on-target locus), and found no significant difference between the CRISPRoff- and PBS-treated mice (FIG. 8E). These results together demonstrated that the epigenetic-mediated gene silencing induced few sgRNA-independent and sgRNA-dependent off-target effects in vivo.

Altogether, the results demonstrate that the EPICAS (CRISPRoff) system could efficiently suppress the expression of the targeted gene Pcsk9 by up to 98% in mouse livers, with higher efficacy than existing drugs (statins, antibodies, and siRNAs) and current gene editing techniques using CRISPR/Cas9 or base editors (8-12). The cleavage-free epigenome editing with the EPICAS (CRISPRoff) system mitigates potential risks of unwanted DNA repair-mediated editting at the targeted loci, which is beneficial for human therapeutic design. It also induced no detectable off-target DNA methylation and alteration in gene expression. Such CRISPRoff-induced methylation could be reversed by CRISPR-mediated de-methylation tools (4). Notably, the CRISPRoff-dependent downregulation of targeted genes persisted after many rounds of cell divisions with transient delivery of CRISPRoff mRNA. In vivo delivery of gene editing tools with LNPs may be preferable than AAVs, since prolonged expression of editing tools by AAV delivery is not necessary nor desirable. Transient LNP-mediated mRNA delivery avoids off-target effects caused by prolonged presence of the editors, and other side-effects of AAV such as immune responses and genomic integration of editing tools (16). Finally, epigenome editing-induced Pcsk9 silencing and blood LDL-C reduction is robust and durable, offering a potential therapeutic strategy for the treatment of FH. Such approaches may also be applicable to therapeutic treatment of other chronic diseases.

### Example 5: Monkey PCSK9 Gene Silencing in Monkey Cells

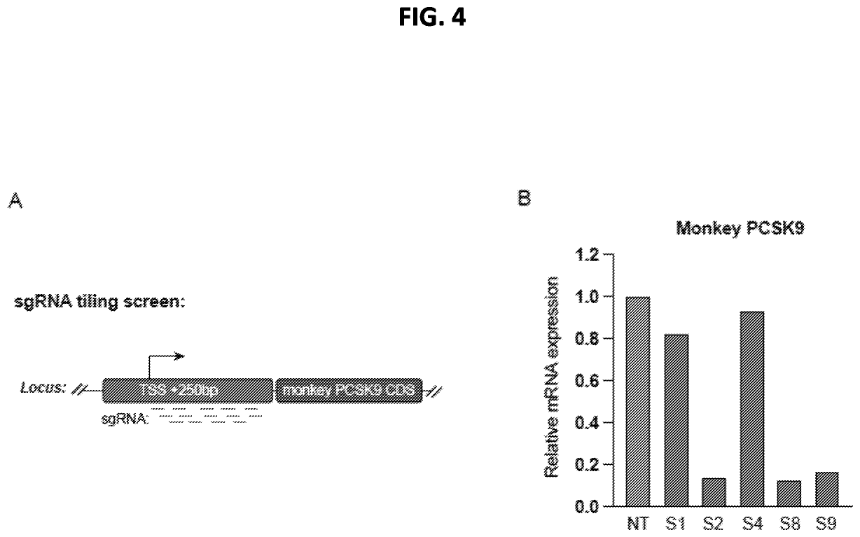

Multiple sgRNAs were designed to target the region within the 250 bp upstream and downstream of the transcription start site (TSS) of the monkey Pcsk9 gene (FIG. 4A).

Individual sgRNA plasmids were co-transfected with the catalytic protein (DNMT3A-DNMT3L-dCas9-KRAB) plasmid into monkey cells. RT-QPCR experiments were performed to evaluate the mRNA expression level of monkey Pcsk9. 3 out of 5 sgRNAs that were tested showed significantly down-regulated expression of Pcsk9 in monkey cells (FIG. 4B). Cells transfected with S2, S8 or S9 sgRNAs resulted in about 90% down regulation of monkey Pcks9.

### Example 6: Human PCSK9 Gene Silencing in Human Cell Lines

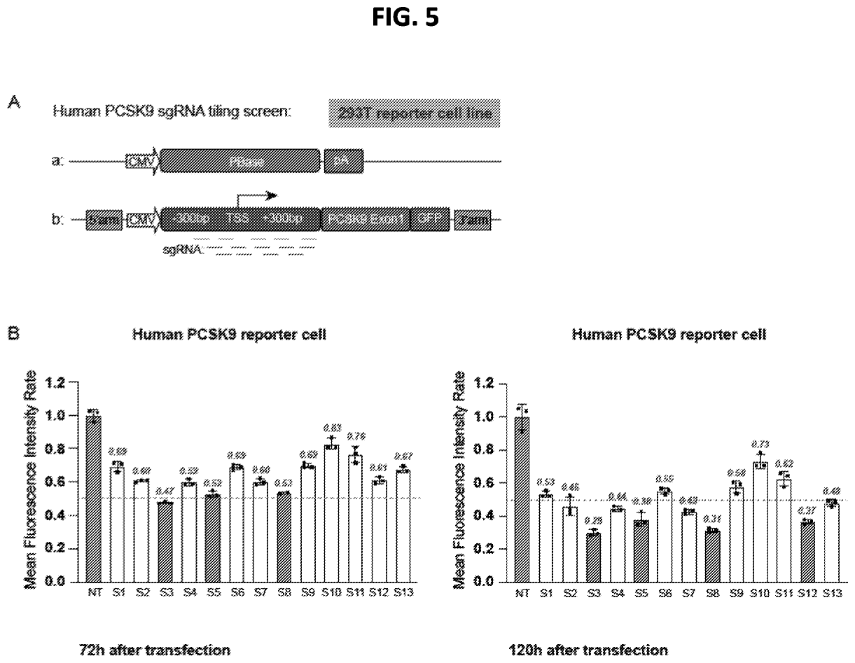

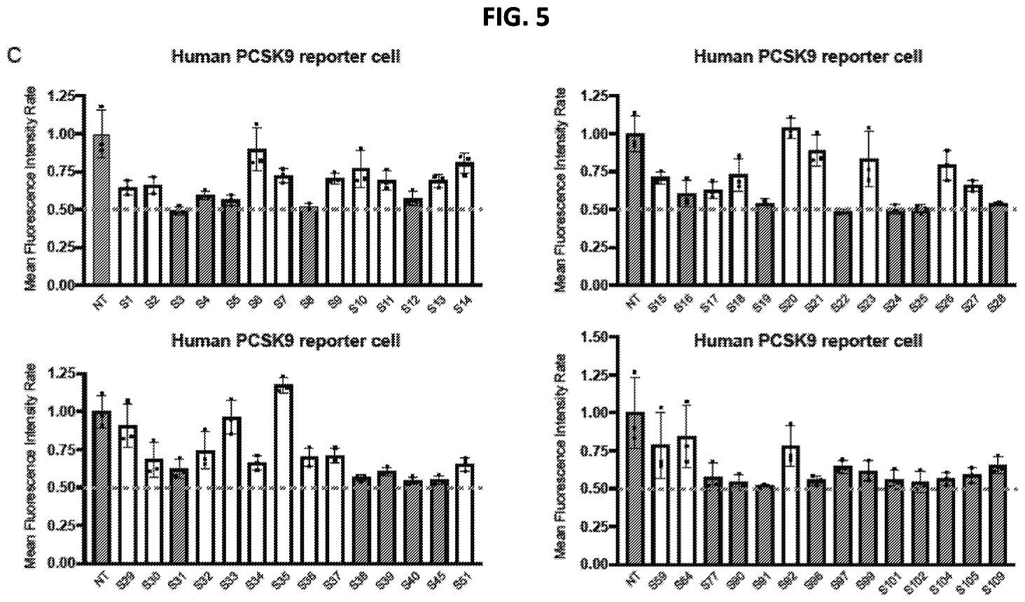

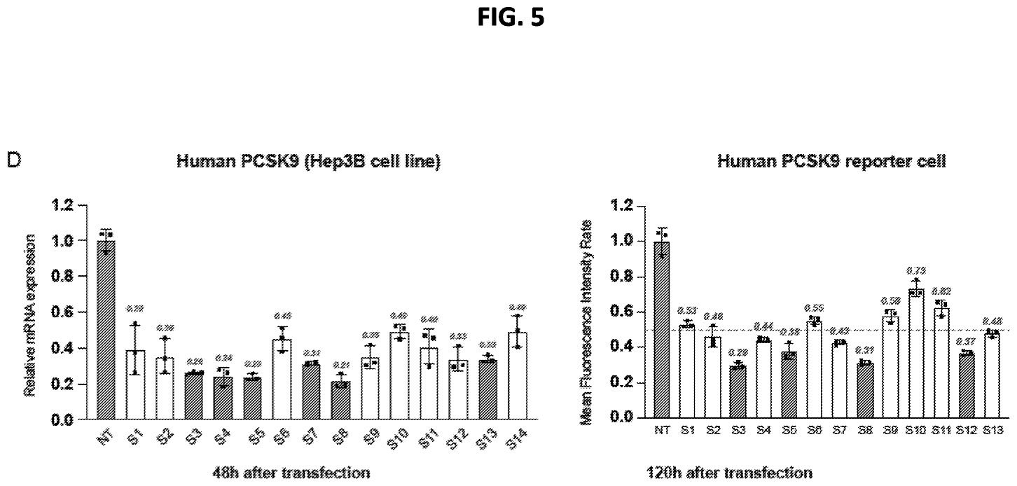

A reporter cell line was constructed in order to test the efficacy of PCKS9 gene silencing in human cell lines. A plasmid was constructed to have a CMV promoter driven cassette, where the cassette had the following elements in the 5′ to 3′ direction: 5′-pCMV-300 bp-TSS-+300 bp-PCSK9 exon1-2A-GFP-3′. In this reporter system, the CMV promoter drives the expression of PCSK9 and GFP fluorescence. If PCSK9 is silenced, the transcription of GFP is terminated. Together with PiggyBac transposase (PBase) plasmid, the reporter plasmid was transfected into the HEK293T cells. Cells with successful reporter cassette integration were sorted by FACS according to the expression of GFP fluorescence (FIG. 5A).

109 sgRNAs were designed to target the regions 300 bp upstream and 300 bp downstream of PCSK9 TSS. Plasmids were constructions to encode each one of the sgRNAs. Individual sgRNA plasmids co-transfected into the human reporter cell lines together with the plasmid encoding the fusion molecule. Decrease of averaged GFP intensity rate at 72 h and 120 h after transfection were analyzed. The general decrease of GFP intensity rate indicated the sensitivity of the reporting system (FIG. 5B). The decreased GFP intensity rate was maintained for 120 h after transfection. Many of the sgRNAs showed much lower GFP intensity rate at 120 h compared with that at 72 h after transfection. These results suggested the efficacy and persistence of EPICAS system in human cell lines.

Next, the experiment was repeated using each of the sgRNAs and the averaged GFP intensity rate was measured at the 72 h timepoint after transfection for comparison (FIG. 5C). More than half of the designed sgRNAs showed significant reduction of averaged fluorescence intensity rate, suggesting that the EPICAS system could induce targeted knock down of PCSK9 expression in human cells.

Next, the use of EPICAS system in the silencing of endogenous PCSK9 expression was tested in human Hep3B cell lines. A plasmid encoding the fusion molecule was co-transfected with various sgRNA plasmids into the Hep3B cells. The mRNA expression level of human PCSK9 was measured by RT-PCR, 48 h post transfection. Six sgRNAs resulted in the highest decrease of PCSK9 expression level (about >65%). These sgRNAs also resulted in about >50% reduction of fluorescence intensity rate in the reporter human cell lines (FIG. 5D). These results showed that EPICAS system could silence the expression of endogenous PCSK9 in human cells with high efficiency and longevity.

Together, these results show that the EPICAS system successfully silenced the expression of PCSK9 in both mouse cells and human cells with high efficiency and persistence supporting silencing PCSK9 gene expression by epigenetic editing. LNPs were successfully used to deliver the EPICAS system in vivo. Accordingly, LNP formulation of the EPICAS system can be used in the treatment of PCSK9 related diseases such as atherosclerotic cardiovascular disease by reducing PCSK9 expression, thereby lowering low-density lipoprotein cholesterol (LDL).

Additional embodiments of the disclosure include the following:

Embodiment 1. A method for reducing or eliminating the expression of a Proprotein Convertase Subtilisin/Kexin type 9 (PCSK9) gene product in a cell, comprising the step of introducing into the cell:
a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
thereby reducing or eliminating the expression of the PCSK9 gene product in the cell.

Embodiment 2. An in vivo method of reducing or eliminating the expression of a PCSK9 gene product in a subject, comprising the step of introducing to a cell of the subject:
a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
thereby reducing or eliminating the expression of the PCSK9 gene product in the subject.

Embodiment 3. A method of reducing low density lipoprotein (LDL) cholesterol in a subject, comprising the step of introducing to a cell of the subject:
a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
thereby reducing LDL cholesterol in the subject.

Embodiment 4. A method for treating or alleviating a symptom of a PCSK9 related disorder in a subject, comprising the step of introducing to a cell of the subject:
a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
thereby treating or alleviating a symptom of a PCSK9 related disorder in the subject.

Embodiment 5. A method of expanding a population of cells with a reduced expression of a PCSK9 gene product comprising the steps of:
i) introducing into a plurality of cells a fusion molecule comprising a least one DNA binding protein and at least one modulator of gene expression, or a nucleic acid sequence encoding the fusion molecule,
   wherein the modulator of gene expression provides a modification of at least one nucleotide near the PCSK9 gene and/or within a PCSK9 regulatory element,
ii) expanding the plurality of cells to produce a plurality of modified cells that have a reduced expression of the PCSK9 gene product,
   wherein the plurality of modified cells has at least 50%, at least 60%, at least 70%, at least 80% or at least 90% reduction in the PCSK9 gene product expression relative to a cell that has not been introduced with the fusion molecule or the nucleic acid sequence, and
   wherein the cell is a liver cell.

Embodiment 6. The method of Embodiment 5, wherein the expression of the PCSK9 gene product is transiently reduced.

Embodiment 7. The method of Embodiment 5, wherein the expression of the PCSK9 gene product is stably reduced.

Embodiment 8. The method of any one of Embodiments 1-7, wherein the PCSK9 regulatory element is a core promoter, a proximal promoter, a distal enhancer, a silencer, an insulator element, a boundary element or a locus control region.

Embodiment 9. The method of any one of Embodiments 1-8, wherein the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the PCSK9 gene.

Embodiment 10. The method of Embodiment 9, wherein the modification of at least one nucleotide near the PCSK9 gene and/or within a PCSK9 regulatory element is located within 1000 bp upstream of the transcription start site of the PCSK9 gene.

Embodiment 11. The method of Embodiment 9, wherein the modification of at least one nucleotide near the PCSK9 gene and/or within a PCSK9 regulatory element is located within 300 bp upstream of the transcription start site of the PCSK9 gene.

Embodiment 12. The method of any one of Embodiments 1-11, wherein the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the PCSK9 gene.

Embodiment 13. The method of Embodiment 12, wherein the modification of at least one nucleotide near the PCSK9 gene and/or within a PCSK9 regulatory element is located within about 300 bp downstream of the transcription start site of the PCSK9 gene.

Embodiment 14. The method of any one of Embodiments 1-8, wherein the modification of at least one nucleotide near the PCSK9 gene and/or within a PCSK9 regulatory element is located within 1000 bp upstream of the transcription start site and within 300 bp downstream of the transcription start site of the PCSK9 gene.

Embodiment 15. The method of any one of Embodiments 1-14, wherein the modification of at least one nucleotide is a DNA methylation.

Embodiment 16. The method of any one of Embodiments 1-15, wherein the at least one modulator of gene expression comprises a DNA methyltransferase (DNMT), a DNA demethylase, a histone methyltransferase, a histone demethylase, or a portion thereof.

Embodiment 17. The method of Embodiment 16, wherein the at least one modulator of gene expression comprises a DNA methyltransferase (DNMT) or a portion thereof.

Embodiment 18. The method of Embodiment 17, wherein the DNA methyltransferase is DNMT3A, DNMT3B, DNMT3L, DNMT1 or DNMT2.

Embodiment 19. The method of Embodiment 18, wherein the DNMT3A comprises the amino acid sequence of SEQ ID NO: 23.

Embodiment 20. The method of Embodiment 18, wherein the DNMT3L comprises the amino acid sequence of SEQ ID NO: 24.

Embodiment 21. The method of any one of Embodiments 1-20, wherein the at least one modulator of gene expression comprises a zinc finger protein-based transcription factor or a portion thereof.

Embodiment 22. The method of Embodiment 21, wherein the zinc finger protein-based transcription factor is Kruppel-associated suppression box (KRAB).

Embodiment 23. The method of Embodiment 22, wherein the KRAB comprises the amino acid sequence of SEQ ID NO: 22.

Embodiment 24. The method of any one of Embodiments 1-23, wherein the at least one modulator of gene expression comprises a DNA methyltransferase or a portion thereof and a zinc finger protein-based transcription factor or a portion thereof.

Embodiment 25. The method of Embodiment 24, wherein the DNA methyltransferase is selected from DNMT3A and DNMT3L and a combination thereof, and the zinc finger protein-based transcription factor is KRAB.

Embodiment 26. The method of any one of Embodiments 1-25, wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

Embodiment 27. The method of Embodiment 26, wherein the at least one DNA binding protein is dCas9.

Embodiment 28. The method of Embodiment 27, wherein the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, a Streptococcus thermophilus (e.g., strain LMD-9) dCas9.

Embodiment 29. The method of Embodiment 27, wherein the dCas9 comprises the amino acid sequence of SEQ ID NO: 1.

Embodiment 30. The method of any one of Embodiments 1-29, wherein the fusion molecule comprises the at least one modulator of gene expression fused to the C-terminus, the N-terminus, or both, of the at least one DNA binding protein.

Embodiment 31. The method of Embodiment 30, wherein the at least one modulator of gene expression is fused directly to the at least one DNA binding protein.

Embodiment 32. The method of Embodiment 30, wherein the at least one modulator of gene expression is fused indirectly with the at least one DNA binding protein via a non-modulator, a second modulator, or a linker.

Embodiment 33. The method of any one of Embodiments 30-32, wherein the fusion molecule comprises a dCas9 fused with a KRAB on the C-terminal end and a DNMT3A and a DNMT3L on the N-terminal end.

Embodiment 34. The method of Embodiment 33, wherein the fusion molecule comprises the amino acid sequence of SEQ ID NO: 97.

Embodiment 35. The method of any one of Embodiments 1-34, wherein the fusion molecule further comprises at least one nuclear localization sequence.

Embodiment 36. The method of Embodiment 35, wherein the at least one nuclear localization sequence is directly fused to the C-terminus, the N-terminus or both of the at least one DNA binding protein.

Embodiment 37. The method of Embodiment 35, wherein the at least one nuclear localization sequence is indirectly fused to the C-terminus, the N-terminus or both of the at least one DNA binding protein via a linker.

Embodiment 38. The method of any one of Embodiments 1-37, wherein the nucleic acid sequence encoding the fusion molecule is a deoxyribonucleic acid (DNA).

Embodiment 39. The method of any one of Embodiments 1-37, wherein the nucleic acid sequence encoding the fusion molecule is a messenger ribonucleic acid (mRNA).

Embodiment 40. The method of any one of Embodiments 1-39, further comprising the step of introducing at least one single guide RNA (sgRNA) that is complementary to a DNA sequence near the PCSK9 gene and/or within a PCSK9 regulatory element, thereby targeting the fusion molecule to the PCSK9 gene or PCSK9 regulatory element, or a DNA encoding the sgRNA.

Embodiment 41. The method of Embodiment 40, wherein the sgRNA comprises the nucleic acid sequence of SEQ ID NOS: 27-95 or 98-108.

Embodiment 42. The method of any one of Embodiments 1-41, wherein the fusion molecule is formulated in a liposome or a lipid nanoparticle.

Embodiment 43. The method of any one of Embodiments 40-41, wherein the fusion molecule and the sgRNA are formulated in a liposome or a lipid nanoparticle.

Embodiment 44. The method of Embodiment 43, wherein the fusion molecule and the sgRNA are formulated in the same liposome or lipid nanoparticle.

Embodiment 45. The method of Embodiment 43, wherein the fusion molecule and the sgRNA are formulated in different liposome or lipid nanoparticle.

Embodiment 46. The method of any one of Embodiments 42-45, wherein the liposome or lipid nanoparticle comprises of ionizable lipids (20%-70%, molar ratio), PEGylated lipids (0%-30%, molar ratio), supporting lipids (5%-50%, molar ratio), and cholesterol (10%-50%, molar ratio).

Embodiment 47. The method of Embodiment 46, wherein the ionizable lipid is selected from a group consisting of pH-responsive ionizable lipids, thermal-responsive ionizable lipids and light-responsive ionizable lipids.

Embodiment 48. The method of any one of Embodiments 1-41, wherein the fusion molecule is formulated in an AAV vector.

Embodiment 49. The method of any one of Embodiments 40-41, wherein the fusion molecule and the sgRNA are formulated in an AAV vector.

Embodiment 50. The method of Embodiment 49, wherein the fusion molecule and the sgRNA are formulated in the same AAV vector.

Embodiment 51. The method of Embodiment 49, wherein the fusion molecule and the sgRNA are formulated in different AAV vectors.

Embodiment 52. The method of any one of Embodiments 1-51, wherein the fusion molecule is delivered to the cell by local injection, systemic infusion, or a combination thereof.

Embodiment 53. The method of any one of Embodiments 2-4 and 8-52, wherein the subject is a human.

Embodiment 54. The method of any one of Embodiments 4 and 8-53, wherein the PCSK9 related disorder is a high atherosclerotic cardiovascular disease.

Embodiment 55. The method of any one of Embodiments 4 and 8-53, wherein the PCSK9 related disorder is hypercholesterolemia.

Embodiment 56. The method of any one of Embodiments 1-55, wherein the cell is a hepatocyte.

Embodiment 57. A sgRNA comprising the nucleic acid sequence of any one of SEQ ID NOs: 27-95 or 98-108.

Embodiment 58. A DNA sequence encoding the sgRNA of Embodiment 54.

Embodiment 59. A pharmaceutical composition comprising a fusion molecule comprising a least one DNA binding protein and at least one modulator of gene expression, or a nucleic acid sequence encoding the fusion molecule,
wherein the fusion molecule is targeted to a genomic region near a PCSK9 gene and/or within a PCSK9 regulatory element,
wherein the at least one modulator of gene expression provides a modification of at least one nucleotide near the PCSK9 gene and/or within a PCSK9 regulatory element,
wherein the at least one modulator of gene expression comprises a DNA methyltransferase (DNMT), a DNA demethylase, a histone methyltransferase, a histone demethylase, or a portion thereof, or a zinc finger protein-based transcription factor or a portion thereof, or a combination thereof, and
wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

Embodiment 60. The pharmaceutical composition of Embodiment 59, wherein the PCSK9 regulatory element is a transcription start site, core promoter, a proximal promoter, a distal enhancer, a silencer, an insulator element, a boundary element or a locus control region.

Embodiment 61. The pharmaceutical composition of any one of Embodiments 59-60, wherein the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the PCSK9 gene.

Embodiment 62. The pharmaceutical composition of Embodiment 61, wherein the modification of at least one nucleotide near the PCSK9 gene and/or within a PCSK9 regulatory element is located within 1000 bp upstream of the transcription start site of the PCSK9 gene.

Embodiment 63. The pharmaceutical composition of Embodiment 61, wherein the modification of at least one nucleotide near the PCSK9 gene and/or within a PCSK9 regulatory element is located within 300 bp upstream of the transcription start site of the PCSK9 gene.

Embodiment 64. The pharmaceutical composition of any one of Embodiments 59-63, wherein the modification of at least one nucleotide near the PCSK9 gene and/or within a PCSK9 regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the PCSK9 gene.

Embodiment 65. The pharmaceutical composition of Embodiment 64, wherein the modification of at least one nucleotide near the PCSK9 gene and/or within a PCSK9 regulatory element is located within about 300 bp downstream of the transcription start site of the PCSK9 gene.

Embodiment 66. The pharmaceutical composition of any one of Embodiments 59-65, wherein the modification of at least one nucleotide near the PCSK9 gene and/or within a PCSK9 regulatory element is located within 1000 bp upstream of the transcription start site and within 300 bp downstream of the transcription start site of the PCSK9 gene.

Embodiment 67. The pharmaceutical composition of any one of Embodiments 59-66, wherein the modification of at least one nucleotide is a DNA methylation.

Embodiment 68. The pharmaceutical composition of Embodiment 59-67, wherein the at least one modulator of gene expression comprises a DNA methyltransferase (DNMT) or a portion thereof.

Embodiment 69. The pharmaceutical composition of Embodiment 68, wherein the DNA methyltransferase is DNMT3A, DNMT3B, DNMT3L, DNMT1 or DNMT2.

Embodiment 70. The pharmaceutical composition of Embodiment 69, wherein the DNMT3A comprises the amino acid sequence of SEQ ID NO: 23.

Embodiment 71. The pharmaceutical composition of Embodiment 69, wherein the DNMT3L comprises the amino acid sequence of SEQ ID NO: 24.

Embodiment 72. The pharmaceutical composition of any one of Embodiments 59-71, wherein the at least one modulator of gene expression comprises a zinc-finger protein-based transcription factor or a portion thereof.

Embodiment 73. The pharmaceutical composition of Embodiment 72, wherein the zinc finger protein-based transcription factor is Kruppel-associated suppression box (KRAB).

Embodiment 74. The pharmaceutical composition of Embodiment 73, wherein the KRAB comprises the amino acid sequence of SEQ ID NO: 22.

Embodiment 75. The pharmaceutical composition of any one of Embodiments 59-74, wherein the at least one modulator of gene expression comprises a DNA methyltransferase or a portion thereof and a zinc finger protein-based transcription factor or a portion thereof.

Embodiment 76. The pharmaceutical composition of Embodiment 75, wherein the DNA methyltransferase is selected from DNMT3A and DNMT3L and a combination thereof, and the zinc finger protein-based transcription factor is KRAB.

Embodiment 77. The pharmaceutical composition of any one of Embodiments 59-76, wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

Embodiment 78. The pharmaceutical composition of Embodiment 77, wherein the at least one DNA binding protein is dCas9.

Embodiment 79. The pharmaceutical composition of Embodiment 78, wherein the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, a Streptococcus thermophilus (e.g., strain LMD-9) dCas9.

Embodiment 80. The pharmaceutical composition of Embodiment 78, wherein the dCas9 comprises the amino acid sequence of SEQ ID NO: 1.

Embodiment 81. The pharmaceutical composition of any one of Embodiments 59-80, wherein the fusion molecule comprises the at least one modulator of gene expression fused to the C-terminus, the N-terminus, or both, of the at least one DNA binding protein.

Embodiment 82. The pharmaceutical composition of Embodiment 81, wherein the at least one modulator of gene expression is fused directly to the at least one DNA binding protein.

Embodiment 83. The pharmaceutical composition of Embodiment 81, wherein the at least one modulator of gene expression is fused indirectly with the at least one DNA binding protein via a non-modulator, a second modulator, or a linker.

Embodiment 84. The pharmaceutical composition of Embodiment 81-83, wherein the fusion molecule comprises a dCas9 fused with a KRAB on the C-terminal end and a DNMT3A and a DNMT3L on the N-terminal end.

Embodiment 85. The pharmaceutical composition of Embodiment 84, wherein the fusion molecule comprises the amino acid sequence of SEQ ID NO: 97.

Embodiment 86. The pharmaceutical composition of any one of Embodiments 59-85, wherein the fusion molecule further comprises at least one nuclear localization sequence.

Embodiment 87. The pharmaceutical composition of Embodiment 86, wherein the at least one nuclear localization sequence is directly fused to the C-terminus, the N-terminus or both of the at least one DNA binding protein.

Embodiment 88. The pharmaceutical composition of Embodiment 86, wherein the at least one nuclear localization sequence is indirectly fused to the C-terminus, the N-terminus or both of the at least one DNA binding protein via a linker.

Embodiment 89. The pharmaceutical composition of any one of Embodiments 59-88, wherein the nucleic acid sequence encoding the fusion molecule is a deoxyribonucleic acid (DNA).

Embodiment 90. The pharmaceutical composition of any one of Embodiments 59-88, wherein the nucleic acid sequence encoding the fusion molecule is a messenger ribonucleic acid (mRNA).

Embodiment 91. The pharmaceutical composition of any one of Embodiments 59-90, further comprising at least one single guide RNA (sgRNA) that is complementary to a DNA sequence near the PCSK9 gene and/or within a PCSK9 regulatory element.

Embodiment 92. The pharmaceutical composition of Embodiment 91, wherein the sgRNA comprises the nucleic acid sequence of SEQ ID NOS: 27-95 or 98-108.

Embodiment 93. The pharmaceutical composition of any one of Embodiments 59-92, wherein the fusion molecule is packaged in a liposome or a lipid nanoparticle.

Embodiment 94. The pharmaceutical composition of any one of Embodiments 91-92, wherein the fusion molecule and the sgRNA are packaged in a liposome or a lipid nanoparticle.

Embodiment 95. The pharmaceutical composition of Embodiment 94, wherein the fusion molecule and the sgRNA are packaged in the same liposome or lipid nanoparticle.

Embodiment 96. The pharmaceutical composition of Embodiment 94, wherein the fusion molecule and the sgRNA are packaged in a different liposome or lipid nanoparticle.

Embodiment 97. The pharmaceutical composition of any one of Embodiments 93-96, wherein the liposome or the lipid nanoparticle comprises of ionizable lipids (20%-70%, molar ratio), PEGylated lipids (0%-30%, molar ratio), supporting lipids (5%-50%, molar ratio), and cholesterol (10%-50%, molar ratio).

Embodiment 98. The pharmaceutical composition of any one of Embodiments 93-97, wherein the ionizable lipid is selected from a group consisting of pH-responsive ionizable lipids, thermal-responsive ionizable lipids and light-responsive ionizable lipids.

Embodiment 99. The pharmaceutical composition of any one of Embodiments 59-92, wherein the fusion molecule is packaged in an AAV vector.

Embodiment 100. The pharmaceutical composition of any one of Embodiments 91-92, wherein the fusion molecule and the sgRNA are packaged in an AAV vector.

Embodiment 101. The pharmaceutical composition of Embodiment 100, wherein the fusion molecule and the sgRNA are packaged in the same AAV vector.

Embodiment 102. The pharmaceutical composition of Embodiment 100, wherein the fusion molecule and the sgRNA are packaged in different AAV vectors.

1. A method for
(a) reducing or eliminating the expression of a Proprotein Convertase Subtilisin/Kexin type 9 (PCSK9) gene product in a cell comprising the step of introducing into the cell:
a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
thereby reducing or eliminating the expression of the PCSK9 gene product in the cell; or
(b) expanding a population of cells with a reduced expression of a PCSK9 gene product comprising the steps of:
i) introducing into a plurality of cells a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
ii) expanding the plurality of cells to produce a plurality of modified cells that have a reduced expression of the PCSK9 gene product,
wherein the plurality of modified cells has at least 50%, at least 60%, at least 70%, at least 80% or at least 90% reduction in the PCSK9 gene product expression relative to a cell that has not been introduced with the fusion molecule or the nucleic acid sequence, and
wherein the cell is a liver cell: or
(c) (i) treating or alleviating a symptom of a PCSK9 related disorder in a subject or (ii) reducing low density lipoprotein (LDL) cholesterol in a subject, the method comprising the step of introducing to a cell of the subject;
a fusion molecule comprising a least one DNA binding protein and at least one modulator of gene expression, or a nucleic acid sequence encoding the fusion molecule,
wherein the modulator of gene expression provides a modification of at least one nucleotide near the PCSK9 gene and/or within a PCSK9 regulatory element,
thereby treating or alleviating a symptom of a PCSK9 related disorder in the subject.

2.-5. (canceled)

6. The method of claim 1, wherein the PCSK9 regulatory element is a core promoter, a proximal promoter, a distal enhancer, a silencer, an insulator element, a boundary element or a locus control region.

7. The method of claim 1, wherein the modification of at least one nucleotide is a DNA methylation.

8. The method of claim 1, wherein the at least one modulator of gene expression comprises a DNA methyltransferase (DNMT), a DNA demethylase, a histone methyltransferase, a histone demethylase, or a portion thereof.

9. The method of claim 8, wherein the DNMT is (a) DNMT3A comprising the amino acid sequence of SEQ ID NO: 23 or (b) DNMT3L comprising the amino acid sequence of SEQ ID NO: 24.

10. (canceled)

11. The method of claim 1, wherein the at least one modulator of gene expression comprises a zinc finger protein-based transcription factor or a portion thereof, optionally wherein the zinc finger protein-based transcription factor is Kruppel-associated suppression box (KRAB): optionally wherein the KRAB comprises the amino acid sequence of SEQ ID NO: 22.

12. (canceled)

13. (canceled)

14. The method of claim 1, wherein the at least one modulator of gene expression comprises a DNA methyltransferase or a portion thereof and a zinc finger protein-based transcription factor or a portion thereof, optionally wherein the DNA methyltransferase is selected from DNMT3A and DNMT3L and a combination thereof, and the zinc finger protein-based transcription factor is KRAB.

15. (canceled)

16. The method of claim 1, wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease optionally wherein the at least one DNA binding protein is dCas9; optionally wherein the dCas9 comprises the amino acid sequence of SEQ ID NO: 1.

17. (canceled)

18. (canceled)

19. The method of claim 1, wherein the fusion molecule comprises a dCas9 fused with a KRAB on the C-terminal end and a DNMT3A and a DNMT3L on the N-terminal end optionally wherein the fusion molecule comprises the amino acid sequence of SEQ ID NO: 97.

20. (canceled)

21. The method of claim 1, further comprising the step of introducing at least one single guide RNA (sgRNA) that is complementary to a DNA sequence near the PCSK9 gene and/or within a PCSK9 regulatory element, thereby targeting the fusion molecule to the PCSK9 gene or PCSK9 regulatory element, or a DNA encoding the sgRNA, optionally wherein the sgRNA comprises the nucleic acid sequence of SEQ ID NOS: 27-95 or 98-108.

22. (canceled)

23. A sgRNA comprising the nucleic acid sequence of any one of SEQ ID NOs: 27-95 or 98-108.

24. A DNA sequence encoding the sgRNA of claim 23.

25. A pharmaceutical composition comprising a fusion molecule comprising a least one DNA binding protein and at least one modulator of gene expression, or a nucleic acid sequence encoding the fusion molecule,
wherein the fusion molecule is targeted to a genomic region near a PCSK9 gene and/or within a PCSK9 regulatory element,
wherein the at least one modulator of gene expression provides a modification of at least one nucleotide near the PCSK9 gene and/or within a PCSK9 regulatory element,
wherein the at least one modulator of gene expression comprises a DNA methyltransferase (DNMT), a DNA demethylase, a histone methyltransferase, a histone demethylase, or a portion thereof, or a zinc finger protein-based transcription factor or a portion thereof, or a combination thereof, and
wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

26. The pharmaceutical composition of claim 25, wherein the PCSK9 regulatory element is a transcription start site, core promoter, a proximal promoter, a distal enhancer, a silencer, an insulator element, a boundary element or a locus control region.

27. (canceled)

28. The pharmaceutical composition of claim 25, wherein the at least one modulator of gene expression comprises a DNA methyltransferase (DNMT) or a portion thereof, optionally wherein the DNMT is (a) DNMT3A comprising the amino acid sequence of SEQ ID NO: 23 or (b) DNMT3L comprising the amino acid sequence of SEQ ID NO: 24.

29. (canceled)

30. (canceled)

31. The pharmaceutical composition of claim 25, wherein the at least one modulator of gene expression comprises a zinc-finger protein-based transcription factor or a portion thereof, optionally wherein the zinc finger protein-based transcription factor is Kruppel-associated suppression box (KRAB), optionally wherein the KRAB comprises the amino acid sequence of SEQ ID NO: 22.

32. (canceled)

33. (canceled)

34. The pharmaceutical composition of claim 25, wherein the at least one modulator of gene expression comprises a DNA methyltransferase or a portion thereof and a zinc finger protein-based transcription factor or a portion thereof, optionally wherein the DNA methyltransferase is selected from DNMT3A and DNMT3L and a combination thereof, and the zinc finger protein-based transcription factor is KRAB.

35. (canceled)

36. The pharmaceutical composition of claim 25, wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease optionally wherein the at least one DNA binding protein is dCas9, optionally wherein the dCas9 comprises the amino acid sequence of SEQ ID NO: 1.

37. (canceled)

38. (canceled)

39. The pharmaceutical composition of claim 25, wherein the fusion molecule comprises a dCas9 fused with a KRAB on the C-terminal end and a DNMT3A and a DNMT3L on the N-terminal end optionally wherein the fusion molecule comprises the amino acid sequence of SEQ ID NO: 97.

40. (canceled)

41. The pharmaceutical composition of claim 25, further comprising at least one single guide RNA (sgRNA) that is complementary to a DNA sequence near the PCSK9 gene and/or within a PCSK9 regulatory element, optionally wherein the sgRNA comprises the nucleic acid sequence of SEQ ID NOS: 27-95 or 98-108.

42. (canceled)
